SEQUENCE LISTING (99 sequences)

SEQ ID NO: 1 - RNA, 21 nt, synthetic construct
  source 1..21: mol_type = other RNA
tcttcaagctcgctgccagca

SEQ ID NO: 2 - RNA, 21 nt, synthetic construct
  source 1..21: mol_type = other RNA
ttcttcaagctcgctgccagc

SEQ ID NO: 3 - RNA, 21 nt, synthetic construct
  source 1..21: mol_type = other RNA
caagctcgctgccagcatcgg

SEQ ID NO: 4 - RNA, 21 nt, synthetic construct
  source 1..21: mol_type = other RNA
tcaagctcgctgccagcatcg

SEQ ID NO: 5 - RNA, 21 nt, synthetic construct
  source 1..21: mol_type = other RNA
cttcaagctcgctgccagcat

SEQ ID NO: 6 - RNA, 21 nt, synthetic construct
  source 1..21: mol_type = other RNA
ttcaagctcgctgccagcatc

SEQ ID NO: 7 - RNA, 21 nt, synthetic construct
  source 1..21: mol_type = other RNA
tttcttcaagctcgctgccag

SEQ ID NO: 8 - RNA, 21 nt, synthetic construct
  source 1..21: mol_type = other RNA
gattcaatccagtcatccacc

SEQ ID NO: 9 - RNA, 21 nt, synthetic construct
  source 1..21: mol_type = other RNA
tgattcaatccagtcatccac

SEQ ID NO: 10 - RNA, 21 nt, synthetic construct
  source 1..21: mol_type = other RNA
tcaatccagtcatccaccacg

SEQ ID NO: 11 - RNA, 21 nt, synthetic construct
  source 1..21: mol_type = other RNA
aatccagtcatccaccacgga

SEQ ID NO: 12 - RNA, 21 nt, synthetic construct
  source 1..21: mol_type = other RNA
ttcaatccagtcatccaccac

SEQ ID NO: 13 - RNA, 21 nt, synthetic construct
  source 1..21: mol_type = other RNA
tgcgatgtccctgtcttgttt

SEQ ID NO: 14 - RNA, 21 nt, synthetic construct
  source 1..21: mol_type = other RNA
gcgatgtccctgtcttgttta

SEQ ID NO: 15 - RNA, 21 nt, synthetic construct
  source 1..21: mol_type = other RNA
tcctttccatcatctgctcgg

SEQ ID NO: 16 - RNA, 21 nt, synthetic construct
  source 1..21: mol_type = other RNA
cctttccatcatctgctcggt

SEQ ID NO: 17 - RNA, 21 nt, synthetic construct
  source 1..21: mol_type = other RNA
ctcctttccatcatctgctcg

SEQ ID NO: 18 - RNA, 21 nt, synthetic construct
  source 1..21: mol_type = other RNA
cctcctttccatcatctgctc

SEQ ID NO: 19 - RNA, 21 nt, synthetic construct
  source 1..21: mol_type = other RNA
cctcctttccatcatctgctc

SEQ ID NO: 20 - RNA, 21 nt, synthetic construct
  source 1..21: mol_type = other RNA
ccctcctttccatcatctgct

SEQ ID NO: 21 - RNA, 21 nt, synthetic construct
  source 1..21: mol_type = other RNA
  modified_base 1..21: mod_base = OTHER; note = Phosphorothioate linkage
tcttcaagctcgctgccagca

SEQ ID NO: 22 - RNA, 21 nt, synthetic construct
  source 1..21: mol_type = other RNA
  modified_base 1..21: mod_base = OTHER; note = Phosphorothioate linkage
ttcttcaagctcgctgccagc

SEQ ID NO: 23 - RNA, 21 nt, synthetic construct
  source 1..21: mol_type = other RNA
  modified_base 1..21: mod_base = OTHER; note = Phosphorothioate linkage
caagctcgctgccagcatcgg

SEQ ID NO: 24 - RNA, 21 nt, synthetic construct
  source 1..21: mol_type = other RNA
  modified_base 1..21: mod_base = OTHER; note = Phosphorothioate linkage
tcaagctcgctgccagcatcg

SEQ ID NO: 25 - RNA, 21 nt, synthetic construct
  source 1..21: mol_type = other RNA
  modified_base 1..21: mod_base = OTHER; note = Phosphorothioate linkage
cttcaagctcgctgccagcat

SEQ ID NO: 26 - RNA, 21 nt, synthetic construct
  source 1..21: mol_type = other RNA
  modified_base 1..21: mod_base = OTHER; note = Phosphorothioate linkage
ttcaagctcgctgccagcatc

SEQ ID NO: 27 - RNA, 21 nt, synthetic construct
  source 1..21: mol_type = other RNA
  modified_base 1..21: mod_base = OTHER; note = Phosphorothioate linkage
tttcttcaagctcgctgccag

SEQ ID NO: 28 - RNA, 21 nt, synthetic construct
  source 1..21: mol_type = other RNA
  modified_base 1..21: mod_base = OTHER; note = Phosphorothioate linkage
gattcaatccagtcatccacc

SEQ ID NO: 29 - RNA, 21 nt, synthetic construct
  source 1..21: mol_type = other RNA
  modified_base 1..21: mod_base = OTHER; note = Phosphorothioate linkage
tgattcaatccagtcatccac

SEQ ID NO: 30 - RNA, 21 nt, synthetic construct
  source 1..21: mol_type = other RNA
  modified_base 1..21: mod_base = OTHER; note = Phosphorothioate linkage
tcaatccagtcatccaccacg

SEQ ID NO: 31 - RNA, 21 nt, synthetic construct
  source 1..21: mol_type = other RNA
  modified_base 1..21: mod_base = OTHER; note = Phosphorothioate linkage
aatccagtcatccaccacgga

SEQ ID NO: 32 - RNA, 21 nt, synthetic construct
  source 1..21: mol_type = other RNA
  modified_base 1..21: mod_base = OTHER; note = Phosphorothioate linkage
ttcaatccagtcatccaccac

SEQ ID NO: 33 - RNA, 21 nt, synthetic construct
  source 1..21: mol_type = other RNA
  modified_base 1..21: mod_base = OTHER; note = Phosphorothioate linkage
tgcgatgtccctgtcttgttt

SEQ ID NO: 34 - RNA, 21 nt, synthetic construct
  source 1..21: mol_type = other RNA
  modified_base 1..21: mod_base = OTHER; note = Phosphorothioate linkage
gcgatgtccctgtcttgttta

SEQ ID NO: 35 - RNA, 21 nt, synthetic construct
  source 1..21: mol_type = other RNA
  modified_base 1..21: mod_base = OTHER; note = Phosphorothioate linkage
tcctttccatcatctgctcgg

SEQ ID NO: 36 - RNA, 21 nt, synthetic construct
  source 1..21: mol_type = other RNA
  modified_base 1..21: mod_base = OTHER; note = Phosphorothioate linkage
cctttccatcatctgctcggt

SEQ ID NO: 37 - RNA, 21 nt, synthetic construct
  source 1..21: mol_type = other RNA
  modified_base 1..21: mod_base = OTHER; note = Phosphorothioate linkage
ctcctttccatcatctgctcg

SEQ ID NO: 38 - RNA, 21 nt, synthetic construct
  source 1..21: mol_type = other RNA
  modified_base 1..21: mod_base = OTHER; note = Phosphorothioate linkage
cctcctttccatcatctgctc

SEQ ID NO: 39 - RNA, 21 nt, synthetic construct
  source 1..21: mol_type = other RNA
  modified_base 1..21: mod_base = OTHER; note = Phosphorothioate linkage
tccatcatctgctcggtaagg

SEQ ID NO: 40 - RNA, 21 nt, synthetic construct
  source 1..21: mol_type = other RNA
  modified_base 1..21: mod_base = OTHER; note = Phosphorothioate linkage
ccctcctttccatcatctgct

SEQ ID NO: 41 - DNA, 21 bp, synthetic construct
  misc_feature 1..5: note = RNA 2-prime-O-[2-methoxyethyl]
  misc_feature 17..21: note = RNA 2-prime-O-[2-methoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl]
tcttcaagctcgctgccagca

SEQ ID NO: 42 - DNA, 21 bp, synthetic construct
  misc_feature 1..5: note = RNA 2-prime-O-[2-methyoxyethyl]
  misc_feature 17..21: note = RNA 2-prime-O-[2-methyoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methyoxyethyl]
ttcttcaagctcgctgccagc

SEQ ID NO: 43 - DNA, 21 bp, synthetic construct
  misc_feature 1..5: note = RNA 2-prime-O-[2-methyoxyethyl]
  misc_feature 17..21: note = RNA 2-prime-O-[2-methyoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methyoxyethyl]
caagctcgctgccagcatcgg

SEQ ID NO: 44 - DNA, 21 bp, synthetic construct
  misc_feature 1..5: note = RNA 2-prime-O-[2-methoxyethyl]
  misc_feature 17..21: note = RNA 2-prime-O-[2-methoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl]
tcaagctcgctgccagcatcg

SEQ ID NO: 45 - DNA, 21 bp, synthetic construct
  misc_feature 1..5: note = RNA 2-prime-O-[2-methyoxyethyl]
  misc_feature 17..21: note = RNA 2-prime-O-[2-methyoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methyoxyethyl]
cttcaagctcgctgccagcat

SEQ ID NO: 46 - DNA, 21 bp, synthetic construct
  misc_feature 1..5: note = RNA 2-prime-O-[2-methyoxyethyl]
  misc_feature 17..21: note = RNA 2-prime-O-[2-methyoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methyoxyethyl]
ttcaagctcgctgccagcatc

SEQ ID NO: 47 - DNA, 21 bp, synthetic construct
  misc_feature 1..5: note = RNA 2-prime-O-[2-methoxyethyl]
  misc_feature 17..21: note = RNA 2-prime-O-[2-methoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl]
tttcttcaagctcgctgccag

SEQ ID NO: 48 - DNA, 21 bp, synthetic construct
  misc_feature 1..5: note = RNA 2-prime-O-[2-methoxyethyl)
  misc_feature 17..21: note = RNA 2-prime-O-[2-methoxyethyl)
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl)
gattcaatccagtcatccacc

SEQ ID NO: 49 - DNA, 21 bp, synthetic construct
  misc_feature 1..5: note = RNA 2-prime-O-[2-methoxyethyl]
  misc_feature 17..21: note = RNA 2-prime-O-[2-methoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl]
tgattcaatccagtcatccac

SEQ ID NO: 50 - DNA, 21 bp, synthetic construct
  misc_feature 1..5: note = RNA 2-prime-O-[2-methoxyethyl]
  misc_feature 17..21: note = RNA 2-prime-O-[2-methoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl]
tcaatccagtcatccaccacg

SEQ ID NO: 51 - DNA, 21 bp, synthetic construct
  misc_feature 1..5: note = RNA 2-prime-O-[2-methoxyethyl]
  misc_feature 17..21: note = RNA 2-prime-O-[2-methoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl]
aatccagtcatccaccacgga

SEQ ID NO: 52 - DNA, 21 bp, synthetic construct
  misc_feature 1..5: note = RNA 2-prime-O-[2-methoxyethyl]
  misc_feature 17..21: note = RNA
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl]
ttcaatccagtcatccaccac

SEQ ID NO: 53 - DNA, 21 bp, synthetic construct
  misc_feature 1..5: note = RNA 2-prime-O-[2-methoxyethyl]
  misc_feature 17..21: note = RNA 2-prime-O-[2-methoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl]
tgcgatgtccctgtcttgttt

SEQ ID NO: 54 - DNA, 21 bp, synthetic construct
  misc_feature 1..5: note = RNA 2-prime-O-[2-methoxyethyl]
  misc_feature 17..21: note = RNA 2-prime-O-[2-methoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl]
gcgatgtccctgtcttgttta

SEQ ID NO: 55 - DNA, 21 bp, synthetic construct
  misc_feature 1..5: note = RNA 2-prime-O-[2-methoxyethyl]
  misc_feature 17..21: note = RNA 2-prime-O-[2-methoxyethyl]
  misc_feature 6..16: note = DNA
  source 1..21: mol_type = other DNA
  modified_base 1..21: mod_base = OTHER; note = Phosphorothioate linkage
tcctttccatcatctgctcgg

SEQ ID NO: 56 - DNA, 21 bp, synthetic construct
  misc_feature 1..5: note = RNA 2-prime-O-[2-methoxyethyl]
  misc_feature 17..21: note = RNA 2-prime-O-[2-methoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl]
cctttccatcatctgctcggt

SEQ ID NO: 57 - DNA, 21 bp, synthetic construct
  misc_feature 1..5: note = RNA 2-prime-O-[2-methoxyethyl]
  misc_feature 17..21: note = RNA 2-prime-O-[2-methoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl]
ctcctttccatcatctgctcg

SEQ ID NO: 58 - DNA, 21 bp, synthetic construct
  misc_feature 1..5: note = RNA 2-prime-O-[2-methoxyethyl]
  misc_feature 17..21: note = RNA 2-prime-O-[2-methoxyethyl]
  misc_feature 6..21: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl]
cctcctttccatcatctgctc

SEQ ID NO: 59 - DNA, 21 bp, synthetic construct
  misc_feature 1..5: note = RNA 2-prime-O-[2-methoxyethyl]
  misc_feature 17..21: note = RNA 2-prime-O-[2-methoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl]
tccatcatctgctcggtaagg

SEQ ID NO: 60 - DNA, 21 bp, synthetic construct
  misc_feature 1..5: note = RNA 2-prime-O-[2-methoxyethyl]
  misc_feature 17..21: note = RNA 2-prime-O-[2-methoxyethyl]
  misc_feature 6..16: note = DNA
  source 1..21: mol_type = other DNA
  modified_base 1..21: mod_base = OTHER; note = Phosphorothioate linkage
  modified_base 5^6: mod_base = OTHER; note = 2-prime-O-[2-methoxyethyl]
ccctcctttccatcatctgct

SEQ ID NO: 61 - RNA, 22 nt, synthetic construct
  source 1..22: mol_type = other RNA
tgcgatgtccctgtcttgttta

SEQ ID NO: 62 - RNA, 22 nt, synthetic construct
  source 1..22: mol_type = other RNA
  modified_base 1..22: mod_base = OTHER; note = Phosphorothioate linkage
tgcgatgtccctgtcttgttta

SEQ ID NO: 63 - DNA, 22 bp, synthetic construct
  misc_feature 1..6: note = RNA 2-prime-O-[2-methoxyethyl]
  misc_feature 17..22: note = RNA 2-prime-O-[2-methoxyethyl]
  misc_feature 6..16: note = DNA
  source 1..22: mol_type = other DNA
  modified_base 1..21: mod_base = OTHER; note = Phosphorothioate linkage
tgcgatgtccctgtcttgttta

SEQ ID NO: 64 - RNA, 19 nt, synthetic construct
  source 1..19: mol_type = other RNA
tgcgatgtccctgtcttgt

SEQ ID NO: 65 - RNA, 17 nt, synthetic construct
  source 1..17: mol_type = other RNA
tgcgatgtccctgtctt

SEQ ID NO: 66 - RNA, 15 nt, synthetic construct
  source 1..15: mol_type = other RNA
tgcgatgtccctgtc

SEQ ID NO: 67 - RNA, 19 nt, synthetic construct
  source 1..19: mol_type = other RNA
gatgtccctgtcttgttta

SEQ ID NO: 68 - RNA, 17 nt, synthetic construct
  source 1..17: mol_type = other RNA
tgtccctgtcttgttta

SEQ ID NO: 69 - RNA, 15 nt, synthetic construct
  source 1..15: mol_type = other RNA
tccctgtcttgttta

SEQ ID NO: 70 - RNA, 19 nt, synthetic construct
  source 1..19: mol_type = other RNA
  modified_base 1..19: mod_base = OTHER; note = Phosphorothioate linkage
tgcgatgtccctgtcttgt

SEQ ID NO: 71 - RNA, 17 nt, synthetic construct
  source 1..17: mol_type = other RNA
  modified_base 1..17: mod_base = OTHER; note = Phosphorothioate linkage
tgcgatgtccctgtctt

SEQ ID NO: 72 - RNA, 15 nt, synthetic construct
  source 1..15: mol_type = other RNA
  modified_base 1..15: mod_base = OTHER; note = Phosphorothioate linkage
tgcgatgtccctgtc

SEQ ID NO: 73 - RNA, 19 nt, synthetic construct
  source 1..19: mol_type = other RNA
  modified_base 1..19: mod_base = OTHER; note = Phosphorothioate linkage
gatgtccctgtcttgttta

SEQ ID NO: 74 - RNA, 17 nt, synthetic construct
  source 1..17: mol_type = other RNA
  modified_base 1..17: mod_base = OTHER; note = Phosphorothioate linkage
tgtccctgtcttgttta

SEQ ID NO: 75 - RNA, 15 nt, synthetic construct
  source 1..15: mol_type = other RNA
  modified_base 1..15: mod_base = OTHER; note = Phosphorothioate linkage
tccctgtcttgttta

SEQ ID NO: 76 - DNA, 19 bp, synthetic construct
  misc_feature 1..5: note = RNA 2-prime-O-[2-methoxyethyl]
  misc_feature 15..19: note = RNA 2-prime-O-[2-methoxyethyl]
  misc_feature 6..14: note = DNA
  source 1..19: mol_type = other DNA
  modified_base 1..19: mod_base = OTHER; note = Phosphorothioate linkage
tgcgatgtccctgtcttgt

SEQ ID NO: 77 - DNA, 17 bp, synthetic construct
  misc_feature 1..5: note = RNA 2-prime-O-[2-methoxyethyl]
  misc_feature 13..17: note = RNA 2-prime-O-[2-methoxyethyl]
  misc_feature 6..12: note = DNA
  source 1..17: mol_type = other DNA
  modified_base 1..17: mod_base = OTHER; note = Phosphorothioate linkage
tgcgatgtccctgtctt

SEQ ID NO: 78 - DNA, 15 bp, synthetic construct
  misc_feature 1..5: note = RNA 2-prime-O-[2-methoxyethyl]
  misc_feature 11..15: note = RNA 2-prime-O-[2-methoxyethyl]
  misc_feature 6..10: note = DNA
  source 1..15: mol_type = other DNA
  modified_base 1..15: mod_base = OTHER; note = Phosphorothioate linkage
tgcgatgtccctgtc

SEQ ID NO: 79 - DNA, 19 bp, synthetic construct
  misc_feature 1..5: note = RNA 2-prime-O-[2-methoxyethyl]
  misc_feature 15..19: note = RNA 2-prime-O-[2-methoxyethyl]
  misc_feature 6..14: note = DNA
  source 1..19: mol_type = other DNA
  modified_base 1..19: mod_base = OTHER; note = Phosphorothioate linkage
gatgtccctgtcttgttta

SEQ ID NO: 80 - DNA, 17 bp, synthetic construct
  misc_feature 1..5: note = RNA 2-prime-O-[2-methoxyethyl]
  misc_feature 13..17: note = RNA 2-prime-O-[2-methoxyethyl]
  misc_feature 6..12: note = DNA
  source 1..17: mol_type = other DNA
  modified_base 1..17: mod_base = OTHER; note = Phosphorothioate linkage
tgtccctgtcttgttta

SEQ ID NO: 81 - DNA, 15 bp, synthetic construct
  misc_feature 1..5: note = RNA 2-prime-O-[2-methoxyethyl]
  misc_feature 11..15: note = RNA 2-prime-O-[2-methoxyethyl]
  misc_feature 6..10: note = DNA
  source 1..15: mol_type = other DNA
  modified_base 1..15: mod_base = OTHER; note = Phosphorothioate linkage
tccctgtcttgttta

SEQ ID NO: 82 - protein, 1258 aa, Homo sapiens
MITSELPVLQDSTNETTAHSDAGSELEETEVKGKRKRGRPGRPPSTNKKPRKSPGEKSRIEAGIRGAGRGRANGHPQQNGEGEPVTLFEVVKLGKSAMQSVVDDWIESYKQDRDIALLDLINFFIQCSGCRGTVRIEMFRNMQNAEIIRKMTEEFDEDSGDYPLTMPGPQWKKFRSNFCEFIGVLIRQCQYSIIYDEYMMDTVISLLTGLSDSQVRAFRHTSTLAAMKLMTALVNVALNLSIHQDNTQRQYEAERNKMIGKRANERLELLLQKRKELQENQDEIENMMNSIFKGIFVHRYRDAIAEIRAICIEEIGVWMKMYSDAFLNDSYLKYVGWTLHDRQGEVRLKCLKALQSLYTNRELFPKLELFTNRFKDRIVSMTLDKEYDVAVEAIRLVTLILHGSEEALSNEDCENVYHLVYSAHRPVAVAAGEFLHKKLFSRHDPQAEEALAKRRGRNSPNGNLIRMLVLFFLESELHEHAAYLVDSLWESSQELLKDWECMTELLLEEPVQGEEAMSDRQESALIELMVCTIRQAAEAHPPVGRGTGKRVLTAKERKTQIDDRNKLTEHFIITLPMLLSKYSADAEKVANLLQIPQYFDLEIYSTGRMEKHLDALLKQIKFVVEKHVESDVLEACSKTYSILCSEEYTIQNRVDIARSQLIDEFVDRFNHSVEDLLQEGEEADDDDIYNVLSTLKRLTSFHNAHDLTKWDLFGNCYRLLKTGIEHGAMPEQIVVQALQCSHYSILWQLVKITDGSPSKEDLLVLRKTVKSFLAVCQQCLSNVNTPVKEQAFMLLCDLLMIFSHQLMTGGREGLQPLVFNPDTGLQSELLSFVMDHVFIDQDEENQSMEGDEEDEANKIEALHKRRNLLAAFSKLIIYDIVDMHAAADIFKHYMKYYNDYGDIIKETLSKTRQIDKIQCAKTLILSLQQLFNELVQEQGPNLDRTSAHVSGIKELARRFALTFGLDQIKTREAVATLHKDGIEFAFKYQNQKGQEYPPPNLAFLEVLSEFSSKLLRQDKKTVHSYLEKFLTEQMMERREDVWLPLISYRNSLVTGGEDDRMSVNSGSSSSKTSSVRNKKGRPPLHKKRVEDESLDNTWLNRTDTMIQTPGPLPAPQLTSTVLRENSRPMGDQIQEPESEHGSEPDFLHNPQMQISWLGQPKLEDLNRKDRTGMNYMKVRTGVRHAVRGLMEEDAEPIFEDVMMSSRSQLEDMNEEFEDTMVIDLPPSRNRRERAELRPDFFDSAAIIEDDSGFGMPMF

SEQ ID NO: 83 - RNA, 6342 nt, Homo sapiens
  source 1..6342: mol_type = mRNA
gtcgccgaagagcgaacaccccaaacaatcccgaagcgccaccaaaaaaaaaaaaaaaaaaaaaaaaaaaaaaaaaaaaaaaaagaaaaaaaaccccgccggatccgaccgccactttcaaaaccccccaccgctctagaaccgcgggagcttccgtccctgagtagaattcgagggtgtaaagaagaggaaggggaaaaatatcttgtaccagcccaggggtgaagaagcccccggcctgagaaagaaggaggagtgggggaggcgaacagtctcgttgctgcctctgtgtacgctgaggggggaggtggccaccgagtactaaattcacttgggaataaaagaaaaacataagaaaattataagagaaaggaattgtcttagaagaaagaaggcaagccaccattttacccacgtaaatatatgaatatatttctgacattgaggtgttccagaagatgataaagaaatgatagcagctccagaaataccaactgattttaatctactacaggagtcagaaacacatttttcttctgacacagattttgaagatatcgaaggaaaaaaccaaaagcaaggcaaaggcaaaacttgtaaaaaaggcaaaaagggcccagcagaaaagggcaaaggtggaaatggaggaggaaaacctccttctggtccaaaccgaatgaatggtcatcaccaacagaatggagtggaaaacatgatgttgtttgaagttgttaaaatgggcaagagtgctatgcagtcggtggtagatgattggatagaatcatacaagcatgaccgagatatagcacttcttgaccttatcaacttttttattcagtgttcaggctgtaaaggagttgtcacagcagaaatgtttagacatatgcagaactctgagataattcgaaaaatgactgaagaattcgatgaggatagtggagattatccacttaccatggctggtcctcagtggaagaagttcaaatccagtttttgtgaattcattggcgtgttagtacggcaatgtcaatatagtatcatatatgatgagtatatgatggatacagtcatttcacttcttacaggattgtctgactcacaagtcagagcatttcgacatacaagcaccctggcagctatgaagttgatgacagctttggtgaatgtggcactaaatcttagcattaatatggataatacacaaagacaatatgaagcagaacggaataaaatgattggaaaacgagccaatgagaggctagaactcctgctacaaaagcggaaagagcttcaggaaaatcaagatgaaatagaaaatatgatgaatgcaatatttaaaggagtgtttgtacatagataccgtgatgcgatagctgaaattcgagctatttgcattgaagagattggcatttggatgaagatgtatagtgatgcctttcttaatgacagttatttaaaatatgttggttggactatgcatgataagcaaggtgaagtaagactcaaatgtcttactgctctacaagggctttattataacaaagagcttaattccaaactggaactttttaccagtcggttcaaggatagaattgtgtctatgacccttgacaaagaatatgatgttgcagtacaagcaataaaattactcactcttgttttacagagtagtgaagaagttctcactgcagaagattgtgaaaatgtctatcatctggtttattcagctcaccggccagtagcagtagcagctggagaatttctctacaaaaagctcttcagtcgtagagatccagaggaggatggaatgatgaaaagaagaggaagacaaggtccaaatgccaaccttgttaagacattggtttttttctttctagaaagtgagttacatgagcatgcagcataccttgtggatagcatgtgggactgtgctactgagctgctgaaagactgggaatgtatgaatagcttgttactggaagagccacttagtggagaggaagcactaacagataggcaagagagtgctctgattgaaataatgctttgtaccattagacaagcggctgaatgtcatcctcccgtgggaagagggacaggaaaaagggtgcttacagcaaaggagaagaagacacagttggatgataggacaaaaatcactgagctttttgccgtggcccttcctcagttattagcaaaatactctgtagatgcagaaaaggtgactaacttgttgcagttgcctcagtactttgatttggaaatatataccactggacgattagaaaagcatttggatgccttattgcgacagatccggaatattgtagagaagcacacagatacagatgttttggaagcatgttctaaaacttaccatgcactctgtaatgaagagttcacaatcttcaacagagtagatatttcaagaagtcaactgatagatgaattggcagataaatttaaccggcttcttgaagattttctgcaagagggtgaagaacctgatgaagatgatgcatatcaggtattgtcaacattgaagaggatcactgcttttcataatgcccatgacctttcaaagtgggatttatttgcttgtaattacaaactcttgaaaactggaatcgaaaatggagacatgcctgagcagattgttattcacgcactgcagtgtactcactatgtaatcctttggcaacttgctaagataactgaaagcagctctacaaaggaggacttgctgcgtttaaagaaacaaatgagagtattttgtcagatatgtcaacattacctgaccaacgtgaatactactgttaaggaacaggccttcactattctgtgtgatattttgatgatcttcagccatcagattatgtcaggagggcgtgacatgttagagccattagtgtatacccctgattcttcattgcagtctgagttgctcagctttattttggatcatgtcttcattgaacaggatgatgataataatagtgcagatggtcagcaagaggatgaagccagtaaaattgaagctctgcacaagagaagaaatttacttgcagcattttgtaagctaattgtatatactgtggtggagatgaatacagctgcagatatcttcaaacagtatatgaagtattataatgactatggagatatcatcaaagaaacaatgagtaaaacaaggcagatagacaaaattcagtgtgctaagacccttattctcagtctgcaacagctttttaatgaaatgatacaagaaaatggctataattttgatagatcatcctctacatttagtggcataaaagaacttgctcgacgttttgctttaacttttggacttgatcagttgaaaacaagagaagccattgccatgctacacaaagatggcatagaatttgcttttaaagagcctaatccgcaaggggagagccatccacctttaaatttggcatttcttgatattctgagtgaattttcttctaaactacttcgacaagacaaaagaacagtgtatgtttacttggaaaagttcatgacctttcagatgtcactccgaagagaggatgtgtggcttccactgatgtcttaccgaaattctttgctagctggtggtgatgatgacaccatgtcagtcattagtggaatcagcagccgggggtcaacagtacggagtaaaaaatcaaaaccatctacaggaaaacggaaagtggttgagggcatgcagctttcactcactgaagaaagtagtagtagtgacagtatgtggttaagcagagaacaaacactgcacacccctgttatgatgcagacaccacaactcacctccactattatgagagagcccaaaagattacggcctgaggatagcttcatgagtgtttatccaatgcagactgaacatcatcaaacacctcttgattataacacgcaggtaacatggatgttagctcaaagacaacaagaggaagcaaggcaacagcaggagagagcagcaatgagctatgttaaactgcgaactaatcttcagcatgccattcggcgtggcacaagcctaatggaagatgatgaagagccaattgtggaagatgttatgatgtcctcagaagggaggattgaggatcttaatgagggaatggattttgacaccatggatatagatttgccaccatcaaagaacagacgagagagaacagaactgaagcctgatttctttgatccagcttcaattatggatgaatcagttcttggagtgtcaatgttttaataccagtacacaattaaatctgtggtgaagtcattttctaagtggaagaggaaattttaaagtgtggtagatacagtgaaattctgtacagatttttctctaaggagaatatgacatgcttatgcttaccaagatcaagtgcattgaggggcagttttgtttgcctgaataaacgtaaaggacaagtaaacaatttgatgataagctacagtttttcttagaaagtaaatattttatttatgcgctgttagttggcttttgaatcgattatttcatgcttttttttaaaaaaaaaaaaaaacaaaataacaatctgaagaggcatttggtacagatatgaattctcttacatttatttactggttgtactaaataatgatgacctctgctggatttctgtttacatccagaaaacaatgttaaggatgtatttattcccctaccctgaagaaagtgtaggatagaattgtttttagcattctaaatttaaatgcttaaaacgtcaatcaacaaaactttgttttaaatattgtaattgtggagaaaagtaaacttataagcagaacttttacaattttttcatctaaaagtattttaagatatttttaaaatccaagagcttctctatacttttcagaaatatccagatgcagtgaactgccagaaggtaaccagtctcaaacatgcttatcccattatcaaccctgaaagtttgcttgtcctttaagataaaaatgtaatgttgtgatattccttccagtaatgccactgtattttgtctccaaataaaagaagcttattgtagtatgtttgcagaaaaattctaaacaaaaattatacagcttattagagtgtgggaatagggatctaaattttaaataaaattatatatatatataaattggtgctgattttataattgcgcagtttgtttagttttttcttacttttaaattccaacttaaaattatgaggtttcagaaatatattgaaagtttaacaatgtttaaaaatagaaaagcatgagtgttcatgctttaaaatgatttttaaatttgtattttatattgttttatctatctgtctttgcaagcagtcttcaggttaaagatacttctaacaggttacagtacatttcctctgtatgtaaattagatgggataatagaattcataacccataatattctttgaaagctaagctttaaacttcattttatgtcctttcacaaataaattagtttaaaacagaaagtggctacttgccattttgacatcaactcattttgcgaggcttaggcagctagacatcgtttaaaacaaaatattaacttatattacatgtgtatctatctattgtcagtcgtctctcagttcttgaggtatattattttaatcattccatgccttaatatgcttgcaatacaagaatatcttcagatgggtgaataccaaaaggctttcagtttttagtcagaaatcaagcattgggctgtggtagccaaaaaccataggttagctaaaaagatcatgatacaattattttattaagtcatggttaataacaaatgaatccagacttgtctaacagattttccatcaacaaatattgttatgtgcaaaagtattgcctatgttgttttacacaccactgcattaactagaactgctgagaggactgtatatatgattttaaacctaagttgattttttttctcactcttgaaaggagtacttctttgtgaaagcagttcttacagctttgttttcaaccagctaaaaatgttttatatattactctaacctgttgtcctccacattctattgtcctaattgtactgttttctgatttgtatttatgtcttgagacagtaactttttgaataaaaataaacctacagtatgttgtatgttttctcttgtactcaaagggggagggtggctataaatggtttgcaaatttatatctattatcacatcttttaatgtgtttggggaataatttatagagaataccatcagtttatatttttaataaatcatatgtatttacaatgaaaaaaaaaa

SEQ ID NO: 84 - RNA, 21 nt, synthetic construct
  source 1..21: mol_type = other RNA
  modified_base 1..21: mod_base = OTHER; note = Phosphorothioate linkage
tgcgatgtccctgtcttgttt

SEQ ID NO: 85 - RNA, 20 nt, synthetic construct
  source 1..20: mol_type = other RNA
  modified_base 1..20: mod_base = OTHER; note = Phosphorothioate linkage
tgcgatgtccctgtcttgtt

SEQ ID NO: 86 - RNA, 19 nt, synthetic construct
  source 1..19: mol_type = other RNA
  modified_base 1..19: mod_base = OTHER; note = Phosphorothioate linkage
tgcgatgtccctgtcttgt

SEQ ID NO: 87 - RNA, 18 nt, synthetic construct
  source 1..18: mol_type = other RNA
  modified_base 1..18: mod_base = OTHER; note = Phosphorothioate linkage
tgcgatgtccctgtcttg

SEQ ID NO: 88 - RNA, 17 nt, synthetic construct
  source 1..17: mol_type = other RNA
  modified_base 1..17: mod_base = OTHER; note = Phosphorothioate linkage
tgcgatgtccctgtctt

SEQ ID NO: 89 - RNA, 16 nt, synthetic construct
  source 1..16: mol_type = other RNA
  modified_base 1..16: mod_base = OTHER; note = Phosphorothioate linkage
tgcgatgtccctgtct

SEQ ID NO: 90 - RNA, 15 nt, synthetic construct
  source 1..15: mol_type = other RNA
  modified_base 1..15: mod_base = OTHER; note = Phosphorothioate linkage
tgcgatgtccctgtc

SEQ ID NO: 91 - RNA, 21 nt, synthetic construct
  source 1..21: mol_type = other RNA
  modified_base 1..21: mod_base = OTHER; note = Phosphorothioate linkage
gcgatgtccctgtcttgttta

SEQ ID NO: 92 - RNA, 20 nt, synthetic construct
  source 1..20: mol_type = other RNA
  modified_base 1..20: mod_base = OTHER; note = Phosphorothioate linkage
cgatgtccctgtcttgttta

SEQ ID NO: 93 - RNA, 19 nt, synthetic construct
  source 1..19: mol_type = other RNA
  modified_base 1..19: mod_base = OTHER; note = Phosphorothioate linkage
gatgtccctgtcttgttta

SEQ ID NO: 94 - RNA, 18 nt, synthetic construct
  source 1..18: mol_type = other RNA
  modified_base 1..18: mod_base = OTHER; note = Phosphorothioate linkage
atgtccctgtcttgttta

SEQ ID NO: 95 - RNA, 17 nt, synthetic construct
  source 1..17: mol_type = other RNA
  modified_base 1..17: mod_base = OTHER; note = Phosphorothioate linkage
tgtccctgtcttgttta

SEQ ID NO: 96 - RNA, 16 nt, synthetic construct
  source 1..16: mol_type = other RNA
  modified_base 1..16: mod_base = OTHER; note = Phosphorothioate linkage
gtccctgtcttgttta

SEQ ID NO: 97 - RNA, 15 nt, synthetic construct
  source 1..15: mol_type = other RNA
  modified_base 1..15: mod_base = OTHER; note = Phosphorothioate linkage
tccctgtcttgttta

SEQ ID NO: 98 - RNA, 6062 nt, Homo sapiens
  source 1..6062: mol_type = mRNA
attggcgtgtggaaaatgccaccagatggcgggttaggattgcagctccgttgaaggcgcggcccccgctcccgaacccccggcgaccaccccgtaacaacccccccacatcgggaataacacaccggagacttttggggggaaactaggtcgatggtcggcggcgcccggatgggcagctgaggattgcctttgaggttattttaaaagttttgagttgtacagcacttgattattttgctgcattgtgaaaggacctctccagcaatgattacttcagaattaccagtgttacaggattcaactaatgaaactactgcccattccgatgctggcagcgagcttgaagaaacagaggtcaaaggaaaaagaaaaaggggtcgtcctggccggcctccatctacaaataagaaacctcgaaaatctccaggtgagaagagcagaattgaagctggaattagaggagcaggccgtggaagagctaatggacaccctcaacagaatggggaaggggagcctgtcacattatttgaggtggtgaaactggggaaaagtgcaatgcagtccgtggtggatgactggattgaatcatataaacaagacagggacatcgcacttctggatttaatcaacttttttatccagtgttcaggatgtcgaggtactgtgagaatagagatgtttcgaaatatgcagaatgcagaaatcatcagaaaaatgactgaagaatttgatgaggacagtggtgattatcctcttaccatgcctggacctcagtggaaaaaatttcgttcaaacttttgtgaatttattggagtcctgattcgacagtgtcagtatagcataatttatgatgagtatatgatggacacagtaatctcccttttgacgggtttgtcagactcccaggtcagagcttttaggcatacaagtaccctggctgccatgaagctcatgactgctctggtgaatgttgccttaaacctcagtattcatcaggataatacccagagacaatatgaagccgagagaaataaaatgattgggaagagagccaatgaaaggttggagttactacttcagaaacgcaaagagctgcaagaaaatcaggatgaaatcgaaaatatgatgaactctatttttaagggtatatttgttcatagataccgtgatgctattgctgagattagagccatttgtattgaagaaattggagtatggatgaaaatgtatagtgatgccttcctaaatgacagttacctaaaatatgttggctggactcttcatgacaggcaaggggaagtcaggctgaagtgtttgaaagctctgcagagtctatataccaatagagaattattccccaaattggaactattcactaaccgattcaaggatcgcattgtatcaatgacacttgataaagaatatgatgttgctgtggaagctattcgattggttactctgatacttcatggaagtgaagaagctctttccaatgaagactgtgaaaatgtttaccacttggtgtactcggcacatcgccctgttgctgtggcagctggagagttccttcacaaaaagctatttagcagacatgacccacaagcagaagaagcattagcaaagaggaggggaagaaacagcccgaatggaaacctcattaggatgctggttcttttctttcttgaaagtgagttacatgaacatgcagcctacttggtggacagtttatgggagagctctcaagaactgttgaaagactgggaatgtatgacagagttgctattagaagaacctgttcaaggagaggaagcaatgtctgatcgtcaagagagtgctcttatagagctaatggtttgtacaattcgtcaagctgctgaggcacatcctccagtgggaaggggtaccggcaagagagtgctaactgccaaagaaaggaaaactcaaattgatgatagaaacaaattgactgaacattttattattacacttcctatgttactgtcaaagtattctgcagatgcagagaaggtagcaaacttgctacaaatcccacagtattttgatttagaaatctacagcacaggtagaatggaaaagcatctggatgctttattaaaacagattaagtttgttgtggagaaacacgtagaatcagatgttctagaagcctgcagtaaaacctatagtatcttatgcagtgaagaatataccatccagaacagagttgacatagctcgaagccagctgattgatgagtttgtagatcgattcaatcattctgtggaagacctattgcaagagggagaagaagctgatgatgatgacatttacaatgttctttctacattaaagcggttaacttcttttcacaatgcacatgatctcacaaaatgggatctctttggtaattgctacagattattgaagactggaattgaacatggagccatgccagaacagatagtcgtgcaagcactgcagtgttcccattattcgattctttggcagttggtgaaaattactgatggctctccttccaaagaggatttgttggtattgaggaaaacggtgaaatcctttttggctgtttgccagcagtgcctgtctaatgttaatactccagtgaaagaacaggctttcatgttactctgtgatcttctgatgattttcagccaccaattaatgacaggtggcagagagggccttcagcctttggtgttcaatccagatactggactccaatctgaactcctcagttttgtgatggatcacgtttttattgaccaagacgaggagaaccagagcatggagggtgatgaagaagatgaagctaataaaattgaggccttacataaaagaaggaatctacttgctgctttcagcaaacttatcatttatgacattgttgacatgcatgcagctgcagacatcttcaaacactacatgaagtattacaatgactatggtgatattattaaggaaacactgagtaaaaccaggcagattgataaaattcagtgtgccaagactctcattctcagtttgcaacagttatttaatgaacttgttcaagagcaaggtcccaacctagataggacatctgcccatgtcagtggcattaaagaactggcacgtcgctttgcccttacatttggattggaccagattaagacacgagaagcagttgccacacttcacaaggatggcatagagtttgcatttaaataccaaaatcagaaaggacaagagtatccacctcctaatctggcttttcttgaagtactaagtgaattttcttctaaacttcttcgacaggacaaaaagacagttcattcatacctagagaaattccttaccgagcagatgatggaaaggagggaggatgtatggcttccactcatctcctatagaaattcattagtcactgggggtgaagatgatagaatgtctgtgaacagtggaagtagcagcagcaaaacctcatcagtaaggaataagaaaggacgacctccacttcataaaaaacgagtagaagatgagagtctggataacacatggctaaacaggactgacaccatgattcagactcctggccccctgccagcaccacaactcacatccactgtactgcgggagaacagtcggcccatgggagaccagattcaagaacctgagtctgaacatggttctgaaccagactttttacacaatcctcagatgcagatctcttggttaggccagccgaagttagaagacttaaatcggaaggacagaacaggaatgaactacatgaaagtgagaactggagtgaggcatgctgttcggggtctaatggaggaagatgctgagcccatctttgaagatgtgatgatgtcatcccgaagccagttagaagatatgaatgaagaatttgaggacaccatggttattgatctgcctccatcaagaaatcggcgagagagagctgagctaaggccagacttctttgactctgcagctatcatagaagatgattcaggatttggaatgcctatgttctgaagtctgaagaaaatttacaaatctggaactctattatttagagctagaggcctatatactgtgatagcttgtatggggaaaaacacttttgatgtgatctgatttgttttttaatcaaatgattaaggtcaatccctttttgcagtgacagaagaggagcatgtaaattacccaagggaatgttggtgaatgtcaactcagaaagactgacctgaaaatcatttgtgtcctactattggacttatcccaatacagatgtgtgtgtttttctggagggaggaagaaattttaaatttttaaaacagctgtcaagataaacactgttatacacctgttttatgaaaactcaacattgagtaaaaaaaaacatatttttaactttattttcctgttgtacaatttaaaaaccgttttaacattttgcctttttatgttttaaaagctaaccatttttattaaacctatgagtaagcagctcatcctaattgcgaagagtgttttggagttcactggatttggttgacctttgtggaacacaaataatgaaggagcagaacattgacaagctaagatgaaattctgacatagtacatctctgccaaaaaccacacaccctctgtggatatggatatgaattcccagattttatatactcttgaataaaaggtttatttttatttataagtgggcataaaataagaaatgtccatgcagccatttttccaacagatgctgtacaccgttcattttatatagactagggagattcaaatacagtgcattttctattggtatttgttctgtgcatttttagcaacttctaccagcaaataaagtattctcagtaaaacgaaaatgattctcaagttatcagtttgctgtttttaccacttatttcatgccctgccaaattcaagttacacagacttccattttcttaagataatcaatcatgaagaaatcctttatcaatcattcaaaagtaattttaagtgtaacataactgtgtttacttcccatgcacttaatacccttatgcgctaattttgtgaattaagtttactgattatagaagtatgtgctgcatagaagtctgtgcttagagggtgaagttcctaagcttaccttgaattacagctacatttcagtgttaaatgtgcatattaagaataattcttttggggaaagaaattatgaatcttcaggacagtctacaatggtttagagttacattctgcctagacttttatgacttgctgctattgttttaaaaaccccacttagtctctctcttcctttctgatttctaaagtaagcctcagaatttccaaaccaattcatccacagctgtttctgggctggtttttaaagtagctgcaacagaatcatgaggctttccctttttatcaaatacgaaaaacattttttaaaattctgcacacccagtgatcatcttttgtgcgggaaagcaagatgatgatggatgattttattcatccttttagtaaagacacaaaacatttttctcaacatttgtacagttctgaaaaaaacctggtcaccaaaaatatcttctctgctaattcagcaattcttgggctccagttaggggagctggggcctcactttctcccagaattgtgggcttcactggaagtgaaggtgcaggaatgactggactgtccaccccagccctgcctgcctgtggttttggccagggagcaagccatgaggtgccctggcacatgcacaaattgatcctttgcgtgacagtcttgtatggaaaacagatgctgacagaattgtagactaccatgccacacaaaaaggctaaatatctactccaatgggtttccagttcagtttgaagtcaatcaaatttttgtattttcggtgtctccttgattggttttgctagtaattctgtaaattgtacatttgcaatatgaggttttttttccttttgtacaatttgaaactgatgcttcacctttcctttaataaactattcaaaatca

SEQ ID NO: 99 - protein, 1268 aa, Homo sapiens
MIAAPEIPTDFNLLQESETHFSSDTDFEDIEGKNQKQGKGKTCKKGKKGPAEKGKGGNGGGKPPSGPNRMNGHHQQNGVENMMLFEVVKMGKSAMQSVVDDWIESYKHDRDIALLDLINFFIQCSGCKGVVTAEMFRHMQNSEIIRKMTEEFDEDSGDYPLTMAGPQWKKFKSSFCEFIGVLVRQCQYSIIYDEYMMDTVISLLTGLSDSQVRAFRHTSTLAAMKLMTALVNVALNLSINMDNTQRQYEAERNKMIGKRANERLELLLQKRKELQENQDEIENMMNAIFKGVFVHRYRDAIAEIRAICIEEIGIWMKMYSDAFLNDSYLKYVGWTMHDKQGEVRLKCLTALQGLYYNKELNSKLELFTSRFKDRIVSMTLDKEYDVAVQAIKLLTLVLQSSEEVLTAEDCENVYHLVYSAHRPVAVAAGEFLYKKLFSRRDPEEDGMMKRRGRQGPNANLVKTLVFFFLESELHEHAAYLVDSMWDCATELLKDWECMNSLLLEEPLSGEEALTDRQESALIEIMLCTIRQAAECHPPVGRGTGKRVLTAKEKKTQLDDRTKITELFAVALPQLLAKYSVDAEKVTNLLQLPQYFDLEIYTTGRLEKHLDALLRQIRNIVEKHTDTDVLEACSKTYHALCNEEFTIFNRVDISRSQLIDELADKFNRLLEDFLQEGEEPDEDDAYQVLSTLKRITAFHNAHDLSKWDLFACNYKLLKTGIENGDMPEQIVIHALQCTHYVILWQLAKITESSSTKEDLLRLKKQMRVFCQICQHYLTNVNTTVKEQAFTILCDILMIFSHQIMSGGRDMLEPLVYTPDSSLQSELLSFILDHVFIEQDDDNNSADGQQEDEASKIEALHKRRNLLAAFCKLIVYTVVEMNTAADIFKQYMKYYNDYGDIIKETMSKTRQIDKIQCAKTLILSLQQLFNEMIQENGYNFDRSSSTFSGIKELARRFALTFGLDQLKTREAIAMLHKDGIEFAFKEPNPQGESHPPLNLAFLDILSEFSSKLLRQDKRTVYVYLEKFMTFQMSLRREDVWLPLMSYRNSLLAGGDDDTMSVISGISSRGSTVRSKKSKPSTGKRKVVEGMQLSLTEESSSSDSMWLSREQTLHTPVMMQTPQLTSTIMREPKRLRPEDSFMSVYPMQTEHHQTPLDYNTQVTWMLAQRQQEEARQQQERAAMSYVKLRTNLQHAIRRGTSLMEDDEEPIVEDVMMSSEGRIEDLNEGMDFDTMDIDLPPSKNRRERTELKPDFFDPASIMDESVLGVSMF

(19) United States
(12) Patent Application Publication
(10) Pub. No.: US 2025/0243486 A1
(43) Pub. Date: Jul. 31, 2025

(54) COMPOSITIONS AND METHODS FOR INHIBITING STAG1 EXPRESSION AND USES THEREOF

(71) Applicants: PRESIDENT AND FELLOWS OF HARVARD COLLEGE, Cambridge, MA (US); DANA-FARBER CANCER INSTITUTE, Boston, MA (US)

(72) Inventors: Karen ADELMAN, Cambridge, MA (US); Benjamin MARTIN, Cambridge, MA (US); Geoffrey NELSON, Boston, MA (US); Zuzana TOTHOVA, Cambridge, MA (US)

(73) Assignees: PRESIDENT AND FELLOWS OF HARVARD COLLEGE, Cambridge, MA (US); DANA-FARBER CANCER INSTITUTE, Boston, MA (US)

(21) Appl. No.: 18/833,715

(22) PCT Filed: Jan. 26, 2023
(86) PCT No.: PCT/US2023/061336

§ 371 Date: Jul. 26, 2024

Related U.S. Application Data
(60) Provisional application No. 63/303,192, filed on Jan. 26, 2022.

(51) Int. Cl. C12N 15/113; A61K 31/4184; A61K 31/4439; A61K 31/502; A61K 31/551; A61P 35/02

(52) U.S. Cl. CPC C12N 15/113; A61K 31/4184; A61K 31/4439; A61K 31/502; A61K 31/551; A61P 35/02; C12N 2310/11; C12N 2310/14; C12N 2310/315; C12N 2310/321; C12N 2310/341; C12N 2310/346

(57) ABSTRACT

Described herein are compositions and methods for downregulating the expression of Stromal Antigen 1 (STAG1). The compositions described comprise antisense oligonucleotides (ASOs) having sequences sufficiently complementary to a portion of a STAG1 primary transcript. Compositions comprising ASOs described can be used for the treatment of cancer, including cohesin-deficient cancers and STAG2-deficient cancers.

### CROSS-REFERENCE TO RELATED APPLICATIONS

This application claims benefit under 35 U.S.C. § 119 (e) of U.S. Provisional Application No. 63/303,192 filed Jan. 26, 2022, the contents of which are incorporated herein by reference in their entirety.

### TECHNICAL FIELD

The technology described herein relates to the use of antisense oligonucleotides for the downregulation of Stromal Antigen 1 for the treatment of cancer.

### BACKGROUND

Myeloid malignancies, including myelodysplastic syndromes (MDS) and acute myeloid leukemia (AML), comprise a heterogeneous group of clonal diseases of mutated hematopoietic stem cells. More than 30,000 new MDS cases and 20,000 new AML cases are diagnosed each year in the United States, with a high mortality rate. While younger patients with MDS or AML may be candidates for intensive chemotherapy followed by allogeneic stem cell transplant (the only potentially curative approach for MDS), there are limited therapeutic options for older patients with these conditions, and long-term survival is less than 5%. Thus, new therapies are urgently needed for these devastating diseases.

Genes encoding components of the cohesin complex are mutated in 11% of patients with MDS and 21% of patients with secondary AML. Cohesin mutations do not exhibit hot-spots and generally result in loss-of-function and/or haploinsufficiency. Although mutations in cohesin might be expected to cause defects in chromosome segregation or genome integrity, mutations that affect these functions are likely lethal. Accordingly, cohesin-mediated disease is characterized by an absence of aneuploidy or complex karyotypes, but with dysregulated gene expression that promotes oncogenic transformation.

Cohesin is a multi-subunit protein complex that is essential for sister chromatid cohesin, chromosome organization into looped domains, DNA damage repair and transcription regulation. Cohesin is one of the most frequently mutated protein complexes in cancer, including myeloid malignancies, with recurrent somatic loss-of-function mutations in core components of the cohesin ring. Importantly, cancer-associated mutations in cohesin rarely affect chromosome integrity, but instead selectively impair gene-regulatory functions. However, how cohesin affects gene activity remains enigmatic, offering no clues towards intervention. Cohesin mutations are associated with poor overall survival and there are currently no therapies known to have selective efficacy in cohesin-mutant cancers. There is therefore a need to define the molecular targets and activities of cohesin and to identify targeted therapeutic approaches for the treatment of disease involving cohesin mutations.

### SUMMARY

Cohesin mutations common in myeloid malignancies such as myelodysplastic syndromes (MDS) and acute myeloid leukemia (AML) disrupt RNA splicing. Consequently, cohesin-deficient cancers, including STAG2-deficient cancers, are highly susceptible to inhibitors of mRNA splicing. The inventors found that one of the RNAs affected by splicing inhibitors in STAG2-deficient cells is the STAG1 RNA, which becomes mis-spliced. Where STAG2-deficient cells, but not wild-type cells are dependent upon the STAG1 paralog for survival, treatments that interfere with STAG1 mRNA splicing and/or stability are found to selectively kill STAG2-deficient cells, such as STAG2-deficient cancer cells. Antisense oligonucleotides targeting STAG1 RNA, are described herein. Also described herein are methods of using such oligonucleotides and/or compositions comprising them to selectively kill STAG2 deficient cells, including but not limited to STAG2-deficient myelodysplastic cells, AML cells and other STAG2-deficient cancer cells. The antisense oligonucleotides targeting STAG1 expression are contemplated for use alone or in combination with other anti-cancer agents, including, but not limited to inhibitors of the DNA damage response, including but not limited to PARP inhibitors.

In one aspect, described herein is a composition for downregulating the expression of Stromal Antigen 1 (STAG1), the composition comprising an antisense oligonucleotide having sequence sufficiently complementary to a portion of a STAG1 primary transcript to permit hybridization thereto, wherein the oligonucleotide comprises at least one phosphorothioate backbone modification.

In one embodiment of this and any other aspect described herein, the antisense oligonucleotide comprises an RNA oligonucleotide, a DNA oligonucleotide, or a combination of deoxyribonucleosides and ribonucleosides.

In another embodiment of this and any other aspect described herein, the antisense oligonucleotide comprises at least one deoxyribonucleoside and at least one ribonucleoside.

In another aspect, described herein is a composition for downregulating the expression of Stromal Antigen 1 (STAG1), the composition comprising an antisense oligonucleotide having sequence sufficiently complementary to a portion of a STAG1 primary transcript to permit hybridization thereto, wherein the oligonucleotide comprises at least one deoxyribonucleoside and at least one ribonucleoside.

In one embodiment of this and any other aspect described herein, the antisense oligonucleotide comprises, in 5′ to 3′ order, 1-5 ribonucleosides, 6-10 deoxyribonucleosides, and 1-5 ribonucleosides. In another embodiment of this and any other aspect described herein, the antisense oligonucleotide comprises, in 5′ to 3′ order, 1-5 ribonucleosides, 6-12 deoxyribonucleosides, and 1-5 ribonucleosides.

In another embodiment of this and any other aspect described herein, one or more of the ribonucleosides comprises a 2′-O-methoxyethyl (MOE) modification.

In another embodiment of this and any other aspect described herein, the antisense oligonucleotide comprises at least one phosphorothioate backbone linkage.

In another embodiment of this and any other aspect described herein, the antisense oligonucleotide comprises phosphorothioate bonds between each nucleoside.

In another embodiment of this and any other aspect described herein, the antisense oligonucleotide comprises 15 to 22 nucleosides. In other embodiments, an antisense oligonucleotide useful in the methods and compositions described herein can comprise, for example, 10-50 nucleosides, e.g., 10-45 nucleosides, 10-40 nucleosides, 10-35 nucleosides, 10-30 nucleosides, 10-29 nucleosides, 10-28 nucleosides, 10-27 nucleosides, 10-26 nucleosides, 10-25 nucleosides, 10-24 nucleosides, 10-23 nucleosides, 10-22 nucleosides, 10-21 nucleosides, 10-20 nucleosides, 12-50 nucleosides, 12-45 nucleosides, 12-40 nucleosides, 12-35 nucleosides, 12-30 nucleosides, 12-29 nucleosides, 12-28 nucleosides, 12-27 nucleosides, 12-26 nucleosides, 12-25 nucleosides, 12-24 nucleosides, 12-23 nucleosides, 12-22 nucleosides, 12-21 nucleosides, 12-20 nucleosides, 15-45 nucleosides, 15-40 nucleosides, 15-35 nucleosides, 15-30 nucleosides, 15-29 nucleosides, 15-28 nucleosides, 15-27 nucleosides, 15-26 nucleosides, 15-25 nucleosides, 15-24 nucleosides, 15-23 nucleosides, 15-22 nucleosides, 15-21 nucleosides, or 15-20 nucleosides.

In another embodiment of this and any other aspect described herein, the antisense oligonucleotide comprises one or more sugar modifications selected from 2′-fluoro, 2′-O-methyl, LNA modification (2′-O,4′-methylene modification), constrained ethyl nucleoside modification (2′,4′-constrained 2′-ethyl nucleoside or 2′-O,4′-ethylene nucleoside), 5-methyl cytosine (5-MeC) and phosphorodiamidate morpholino (PMO) modification.

In another embodiment of this and any other aspect described herein, the antisense oligonucleotide comprises sequence permitting hybridization to exon 3 or 5 of the STAG1 RNA transcript. In another embodiment of this and any other aspect described herein, the antisense oligonucleotide comprises sequence permitting hybridization to exon 29.

In some embodiments, the ASO compositions as described herein target elements of the primary transcript that influence mRNA splicing.

In another embodiment of this and any other aspect described herein, the exonic splicing enhancer comprises a binding site for RNA binding protein SRSF2.

In another embodiment of this and any other aspect described herein, the antisense oligonucleotide comprises a sequence selected from those presented in Table 1, which includes SEQ ID NOs 1-81.

In another aspect, described herein is a pharmaceutical formulation comprising an antisense oligonucleotide as described herein and a pharmaceutically acceptable carrier.

In another aspect, described herein is a method of downregulating the expression of STAG1 in a cell, the method comprising contacting the cell with an antisense oligonucleotide as described herein.

In one embodiment of this and any other aspect described herein, the cell is a cohesin mutant cell.

In another embodiment of this and any other aspect described herein, the cell is a STAG2 mutant cell.

In another embodiment of this and any other aspect described herein, the cell is a cancer cell or a myelodysplastic syndrome (MDS) cell. In one embodiment, MDS represents a precursor to acute myeloid leukemia (AML).

In another embodiment of this and any other aspect described herein, the cell is a cancer cell selected from an acute myeloid leukemia (AML) cell, a glioblastoma cell and a bladder cancer cell.

In another embodiment of this and any other aspect described herein, the contacting reduces STAG1 mRNA by at least 50% in the cell.

In another aspect, described herein is a method of selectively killing a cohesin mutant cell, the method comprising contacting the cell with an antisense oligonucleotide as described herein.

In one embodiment of this and any other aspect described herein, the cell is a STAG2 mutant cell.

In another embodiment of this and any other aspect described herein, the cell is a cancer cell or a myelodysplastic syndrome cell.

In another embodiment of this and any other aspect described herein, the cell is a cancer cell selected from an acute myeloid leukemia (AML) cell, a glioblastoma cell and a bladder cancer cell.

In another embodiment of this and any other aspect described herein, the contacting reduces STAG1 mRNA by at least 50% in the cell. In another embodiment, the contacting reduces STAG1 mRNA by at least 20%, at least 30%, at least 40%, at least 50%, at least 60%, at least 70%, at least 80% or at least 90%.

In another aspect, described herein is a method for treating cohesin mutant cancer, the method comprising administering an antisense oligonucleotide as described herein to a subject in need thereof.

In one embodiment of this and any other aspect described herein, the cohesin mutant cancer is STAG2 mutant.

In another embodiment of this and any other aspect described herein, the cohesin mutant cancer is selected from AML, glioblastoma, and bladder cancer.

In another aspect, described herein is a method of treating myelodysplastic syndrome, the method comprising administering an antisense oligonucleotide as described herein to a subject in need thereof.

In one embodiment of this and any other aspect described herein, myelodysplastic syndrome cells are cohesin mutant.

In another embodiment of this and any other aspect described herein, myelodysplastic syndrome cells are STAG2 mutant.

In another embodiment of a method of treating cancer or myelodysplastic syndrome as described herein, the method further comprises administering an inhibitor of the DNA damage response. In one embodiment, the inhibitor of the DNA damage response is a poly (ADP)-ribose polymerase (PARP) inhibitor.

### Definitions

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology, and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The term “hybridize” refers to the annealing of complementary nucleic acids that occurs through nucleobase complementarity. Hybridization is governed by the base sequences involved, with complementary nucleobases forming hydrogen bonds, and the stability of any hybrid being determined by the identity of the base pairs (e.g., G: C base pairs being stronger than A: T base pairs) and the number of contiguous base pairs, with longer stretches of complementary bases forming more stable hybrids.

The term “mismatch” means a nucleobase of a first nucleic acid that is not capable of base pairing with a nucleobase at a corresponding position of a second nucleic acid.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. In some embodiments, include analogs with nucleic acid modifications, including, but not limited to peptide nucleic acids (PNA), bridged nucleic acids (BNA), morpholinos, locked nucleic acids (LNA), glycerol nucleic acids (GNA), threose nucleic acids (TNA), or other synthetic nucleic acids (XNA) described in the art

Nucleic acids can be single stranded or double stranded, or can contain portions of both double stranded and single stranded sequence. The nucleic acid can be DNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods.

A nucleic acid will generally contain phosphodiester bonds between nucleosides, although nucleic acid analogs can be included that can have at least one different linkage, e.g., phosphoramidate, phosphorothioate, phosphorodithioate, or O-methylphosphoroamidite linkages and peptide nucleic acid backbones and linkages. Other analog nucleic acids include those with positive backbones; non-ionic backbones, and non-ribose backbones, including those described in U.S. Pat. Nos. 5,235,033 and 5, 034,506, which are incorporated by reference. Nucleic acids containing one or more non-naturally occurring or modified nucleotides are also included within one definition of nucleic acids. The modified nucleotide analog can be located for example at the 5′-end and/or the 3′-end of the nucleic acid molecule. Representative examples of nucleotide analogs can be selected from sugar- or backbone-modified ribonucleotides. It should be noted, however, that also nucleobase-modified ribonucleotides, i.e. ribonucleotides containing a non-naturally occurring nucleobase instead of a naturally occurring nucleobase such as uridines or cytidines modified at the 5-position, e.g. 5-(2-amino) propyl uridine, 5-bromo uridine; adenines and guanosines modified at the 8-position, e.g. 8-bromo guanosine; deaza nucleotides, e. g. 7 deaza-adenine; O— and N-alkylated nucleotides, e.g. N6-methyl adenine are suitable. The 2′ OH— group can be replaced by a group selected from H. OR, R. halo, SH, SR, NH2, NHR, NR2 or CN, wherein R is C-C6 alkyl, alkenyl or alkynyl and halo is F. Cl, Br or I. Modifications of the ribose-phosphate backbone can be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs can be made.

As used herein the term “exonic splicing enhancer” or “ESE” refers to a nucleic acid sequence motif consisting of 6-8 bases within an exon that directs, or enhances, accurate splicing of heterogeneous nuclear RNA (hnRNA) or pre-mRNA into messenger RNA (mRNA). ESEs commonly occur in both alternative and constitutive exons, where they act as binding sites for Ser/Arg-rich proteins (SR proteins), a family of conserved splicing factors that participate in various steps of the splicing pathway (see, e.g., Gravely, RNA 6:1197-1211 (2000)). SR proteins bind to ESEs, and recruit spliceosomal factors via protein: protein interactions mediated by their serine rich domain and/or by antagonizing the action of nearby splicing silencers. Different SR proteins have different RNA substrate sequence specificities, and a number of classes of ESE consensus motifs have been described (see, e.g., Cartegni et al., Nature Rev. Genet. 3:285-298 (2002), Gravely et al., id, and Fairbrother et al., Science 297:1007-1013 (2002)). ESEs can be identified, for example, using a web resource called ESEfinder—see, e.g., Cartegni et al., Nucl. Acids Res. 31:3568-3571 (2003).

As used herein the term “selectively killing” refers to a given treatment or process which results in the killing of cells expressing or not expressing, as the case may be, one or more particular or target characteristic, to the substantial exclusion of cells that do not have that characteristic. Thus, where a given treatment or process kills cells that are mutant or deficient in STAG2 function, but not cells that have normal STAG2 function, the treatment or process selectively kills the STAG2 mutant or deficient cells. “Substantial exclusion” as used in this context means that≤30% of cells killed did not have the target characteristic, or alternatively, that≤25%, ≤20%, ≤15%, or ≤10% of cells killed did not have the target characteristic, preferably ≤5% of cells killed did not have the target characteristic; more preferably ≤1%, and more preferably still, no killing of cells lacking the target characteristic.

As used herein, the term “PARP inhibitor” refers to a composition, substance, or molecule that blocks or interferes with the enzyme Poly (ADP-ribose) polymerase (PARP). A variety of different PARP inhibitors are known, including but not limited to talazoparib, veleparib, pamiparib, olaparib, rucaparib and niraparib.

The terms “increased”, “increase”, “enhance”, “activate” are all used herein to refer to an increase by a statistically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level

The term “improve” or “improvement,” when applied to a score in a standardized scale or rating, e.g., for disease symptoms or severity, means a statistically significant, favorable change in the scale or rating on that scale.

The term “decrease”, “reduced”, “reduction”, or “inhibit” are all used herein to mean a decrease by a statistically significant amount. In some embodiments, “decrease”, “reduced”, “reduction”, or “inhibit” typically means a decrease by at least 10% as compared to a reference level, for example, a decrease of at least about 20%, or at least about 30%, or at least about 40%, or at least about 50%, or at least about 60%, or at least about 70%, or at least about 80%, or at least about 90%, or at least about 95% as compared to a reference level. As used herein, “reduction” or “inhibition” does not encompass a complete inhibition or reduction as compared to a reference level. “Complete inhibition” is a 100% inhibition as compared to a reference level.

As used herein, a “reference level” refers to the level or value for a given parameter against which one compares the level or value in a given sample or situation to determine whether the level or value has changed in a meaningful way. A reference level can be a level in or from a sample that is not treated to change the parameter. A reference level can alternatively be a level in or from a normal or otherwise unaffected sample. A reference level can alternatively be a level in or from a sample obtained from a subject at a prior time point, for example, prior to a given treatment.

As used herein, an “appropriate control” refers to an untreated, otherwise identical cell or population (e.g., a subject who was not administered an agent described herein, or was administered only a subset of agents described herein, as compared to a non-control cell).

The term “modulation,” when applied to target gene expression refers to altering levels; i.e., an increase or decrease in expression as those terms are defined herein. This modulation can be measured in ways which are routine in the art, for example by Northern blot assay, RNase protection assay or reverse transcriptase PCR for measurement of transcription or splicing products or mRNA, or by Western blot, ELISA or immunoprecipitation assay for protein expression. Effects of antisense oligonucleotides as described herein on target gene expression can also be determined as taught in the examples herein or, for example, in WO2020/191212, which is incorporated herein by reference. Modulation of a target gene, e.g., STAG1, is preferably sufficient to provide a measurable effect on a cell that otherwise expresses the target gene. Where, for example, the modulation decreases expression of STAG1 in a STAG2 deficient cell, the modulation can promote killing of the cell.

As used herein, an “exon” refers to any part of a primary gene transcript that is comprised by the final mature RNA produced by that gene after introns have been removed by RNA splicing. The term exon refers to both the DNA sequence within a gene and to the corresponding sequence in RNA transcripts.

As used herein, an “intron” refers to any nucleotide sequence within a gene or primary transcript of the gene that is removed by RNA splicing during maturation of the final RNA product. The term intron refers to both the DNA sequence within a gene and the corresponding sequence in RNA transcripts.

As used herein, the term “alternative splicing” refers to a regulated process during gene expression that results in a single gene coding for more than one protein or product. In this process, particular exons of a gene may be included within or excluded from the final, processed messenger RNA (mRNA) produced from that gene. In one embodiment, the compositions and methods described herein do not modulate alternative splicing.

The term “therapeutically effective amount” refers to an amount of an ASO or pharmaceutical composition comprising an ASO as described herein that is sufficient to provide a particular beneficial effect when administered to a typical subject in need thereof. An effective amount as used herein would also include an amount sufficient to delay the development of a symptom of a disease, alter the course of a symptom of a disease (for example but not limited to, slow the progression of a symptom of a disease), or reverse a symptom of a disease. Thus, while it is not possible or practical to specify an exact “effective amount” for every situation, for any given case, an appropriate “effective amount” can be determined by one of ordinary skill in the art using only routine experimentation.

In one embodiment, a composition as described herein, e.g., a pharmaceutical composition or formulation, further comprises a pharmaceutically acceptable carrier. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, medium, encapsulating material, manufacturing aid (e.g., lubricant, talc, magnesium, calcium or zinc stearate, or steric acid), or solvent encapsulating material, involved in maintaining the stability, solubility, or activity of, an agent. Each carrier must be “acceptable” in the sense of being compatible with the other ingredients of the formulation and not injurious to the patient. The terms “excipient,” “carrier,” “pharmaceutically acceptable carrier” or the like are used interchangeably herein.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of diseases. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

As used herein, a “subject in need” of treatment for a particular condition can be a subject having that condition, diagnosed as having that condition, or at increased risk of developing that condition.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE FIGURES

FIG. 1 shows cohesin composition and architecture. Cohesin is a ring-shaped complex that consists of SMCIA, SMC3, RAD21, and STAG1 or STAG2. SMCs contain two coiled-coil stretches separated by a flexible globular “hinge” domain. When the Smc1A and Smc3 proteins are folded at their hinge domains, the NTP-binding motif and the DA box present at their amino- and carboxy-terminal globular domains come together to form a functional ATPase. Smc1A and Smc3 interact through their hinges, whereas the kleisin subunit Rad21 bridges their head domains and associates with SA. The outer diameter of the resulting ring-shaped complex is estimated at ˜50 nm and could hold two 10-nm chromatin fibers. The ring is closed by STAG1 or STAG2 proteins, which are paralogs that are mutually exclusive.

FIG. 2 shows a mutation matrix of cohesin subunits in AML depicting cohesin subunits SMC1A, SMC3, RAD21, STAG1, and STAG2.

FIG. 3A-FIG. 3C shows an AML cell model with genetically engineered STAG2 KO can recapitulate selective dependence on STAG1.

FIG. 4A-FIG. 4B show that STAG2 KO renders cell highly sensitive to reduction of STAG1 levels.

FIG. 5 shows that cohesin mutations render AML cells highly sensitive to splicing inhibitors such as H3B-8800.

FIG. 6 shows modifications to the backbone or sugar positions of antisense oligonucleotides (ASOs). Chemical modifications that can impart drug-like properties to oligonucleotides include 2′O-methoxyethyl (MOE), 2′fluoro (F), 2′-O-methyl (OMe), Locked nucleic acid or 2′-O,4′-methylene nucleoside (LNA), constrained ethyl nucleoside also known as 2′,4′-constrained 2′-ethyl nucleoside or 2′-O,4′-ethylene nucleoside (CET), and phosphorodiamidate morpholino (PMO). These modifications can enhance nuclease stability and, for example, affinity for proteins or RNA.

FIG. 7 shows single-stranded phosphorothioate (PS)-modified MOE gapmer ASO (yellow spheres show PS, and pink spheres show MOE modifications). Gapmer refers to an ASO with central region or gap that has DNA characteristics flanked by modified RNA bases. The short central stretch of DNA will form an RNA-DNA hybrid with RNA target. This RNA-DNA hybrid can recruit RNAase H to degrade target RNA.

FIG. 8A-FIG. 8B show uptake of fluorescently labeled ASO in HEK 293T cells.

FIG. 9A-FIG. 9B show uptake of fluorescently labeled ASOs in U937 AML cell line.

FIG. 10A-FIG. 10B show ASOs targeting regions of STAG1 reduce STAG1 expression.

FIG. 11A-FIG. 11B shows reduction of STAG1 protein expression following treatment with STAG1 targeting ASOs.

FIG. 12 shows truncations of SEQ ID NO: 62 STAG1 ASO. Similar truncations of any of the other ASOs in Table 1 are also contemplated, e.g., to evaluate potential impact on optimal ASO function.

T*G*C*G*A*T*G*T*C*C*C*T*G*T*C*T*T*G*T*T*T*A (SEQ ID NO: 62),
T*G*C*G*A*T*G*T*C*C*C*T*G*T*C*T*T*G*T*T*T (SEQ ID NO: 84),
T*G*C*G*A*T*G*T*C*C*C*T*G*T*C*T*T*G*T*T (SEQ ID NO: 85),
T*G*C*G*A*T*G*T*C*C*C*T*G*T*C*T*T*G*T (SEQ ID NO: 86),
T*G*C*G*A*T*G*T*C*C*C*T*G*T*C*T*T*G (SEQ ID NO: 87),
T*G*C*G*A*T*G*T*C*C*C*T*G*T*C*T*T (SEQ ID NO: 88),
T*G*C*G*A*T*G*T*C*C*C*T*G*T*C*T (SEQ ID NO: 89),
T*G*C*G*A*T*G*T*C*C*C*T*G*T*C (SEQ ID NO: 90),
G*C*G*A*T*G*T*C*C*C*T*G*T*C*T*T*G*T*T*T*A (SEQ ID NO: 91),
C*G*A*T*G*T*C*C*C*T*G*T*C*T*T*G*T*T*T*A (SEQ ID NO: 92),
G*A*T*G*T*C*C*C*T*G*T*C*T*T*G*T*T*T*A (SEQ ID NO: 93),
A*T*G*T*C*C*C*T*G*T*C*T*T*G*T*T*T*A (SEQ ID NO: 94),
T*G*T*C*C*C*T*G*T*C*T*T*G*T*T*T*A (SEQ ID NO: 95),
G*T*C*C*C*T*G*T*C*T*T*G*T*T*T*A (SEQ ID NO: 96),
T*C*C*C*T*G*T*C*T*T*G*T*T*T*A (SEQ ID NO: 97).

FIG. 13 shows the optimization of ASO length and sequence. E5-12 and E5-11 were the ASOs that had the best effect, and the remaining ASOs were synthesized as derivatives of those sequences.

FIG. 14 shows HiBit readout using STAG1-HiBit expressing HEK 293T cells following STAG1 targeting ASOs.

FIG. 15 shows STAG1 protein levels following treatment with STAG1 targeting ASOs.

FIG. 16A-FIG. 16B show optimized ASO E5-14 causes selective lethality in STAG2 KO AML cells.

### DESCRIPTION

The compositions and methods described herein relate to the treatment of cancer. Cohesin is one of the most frequently mutated protein complexes in cancer, including myeloid malignancies, with recurrent somatic loss-of-function mutations in core components of the cohesin ring. Cohesin-deficient cancers, including STAG2-deficient cancers, are highly susceptible to inhibitors of mRNA splicing. The inventors found that one of the RNAs affected by splicing inhibitors in STAG2-deficient cells is the STAG1 RNA, which becomes mis-spliced. Where STAG2-deficient cells, but not wild-type cells are dependent upon the STAG1 paralog for survival, treatments that interfere with STAG1 mRNA splicing and/or stability are found to selectively kill STAG2-deficient cells, such as STAG2-deficient cancer cells.

Antisense oligonucleotides targeting STAG1 RNA, including, but not limited to antisense oligonucleotides capable of hybridizing to portions of the STAG1 transcript that influence mRNA splicing efficiency, are described herein. Also described herein are methods of using such oligonucleotides and/or compositions comprising them to selectively kill STAG2 deficient cells, including but not limited to STAG2-deficient myelodysplastic cells, AML cells and other STAG2-deficient cancer cells. The antisense oligonucleotides targeting STAG1 expression are contemplated for use alone or in combination with other anti-cancer agents, including, but not limited to inhibitors of the DNA damage response, including but not limited to PARP inhibitors.

The following describes considerations to permit one of ordinary skill in the art to make and use the subject technology.

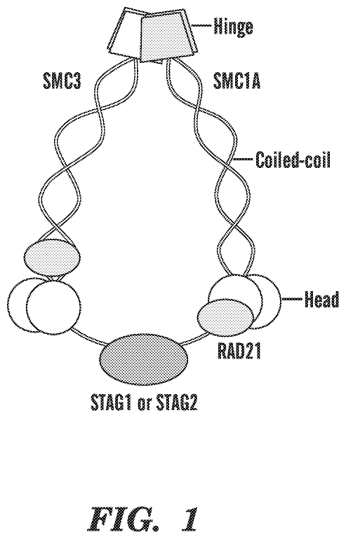

Cohesin is a multi-subunit protein complex that forms a ring-like structure around DNA, with three structural subunits, SMC1A, SMC3 and RAD21 bound to either STAG1 or STAG2 proteins (FIG. 1). Cohesin is one of the most frequently mutated protein complexes in cancer (see, e.g., Leiserson et al., Nat. Genet. (2015) doi: 10.1038/ng.3168, Lawrence et al., Nature (2014) doi: 10.1038/nature12912, and Losada et al., Nat. Rev. cancer (2014) doi: 10.1038/nrc3743). Moreover, the cohesin subunit STAG2 is 1 of only 12 human genes to be significantly mutated in four or more distinct types of human cancer (Lawrence et al., id., Cucco & Musio, Am. J. Med. Genet. Part C: Seminars in Medical genetics (2016) doi: 10.1002/ajmg.c.31492), with recurrent mutations in myeloid malignancies, glioblastoma, breast cancer, bladder cancer, melanoma and Ewing sarcoma (Leiserson et al., id., Lawrence et al., id., Cucco & Musio, id., Viny & Levine, Curr. Opin. Hematol. 25:61-66 (2018)). Despite this prevalence, there are no targeted therapeutic approaches available to treat disease involving cohesin mutations.

A cohesin mutant cell includes a cell in which one or more subunits of the cohesin complex is mutated, such that cohesin function is negatively affected or deficient. As used herein, the term “cohesin deficient” means that the activity or expression of the cohesin complex or a subunit thereof is reduced by at least 20% relative to wild-type, e.g, reduced by at least 20%, at least 30%, at least 40%, at least 50%, at least 60%, at least 70%, at least 80%, at least 90% or more relative to wild-type expression or activity. Subunits of the cohesin complex include SMC3, SMCIA, RAD21, and STAG1 or STAG2 (Stromal Antigen 2), which are mutually exclusive paralogs.

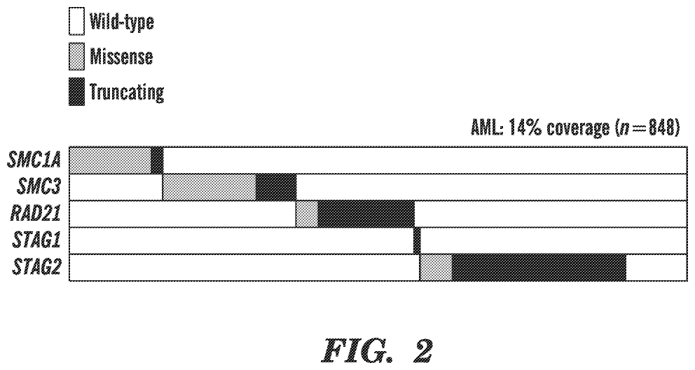

As used herein the term “cohesin mutant cell” refers to a cell with a genotype that differs from its original genotype due to changes in the DNA of any of the genes encoding cohesin subunits, e.g., RAD21, SMC1A, SMC3, and/or STAG1/STAG2). A cohesin mutant cell will be defective with respect to the expression or function of one or more of these subunits, i.e., expression or function of one or more of these subunits will be absent or will be reduced as that term is defined herein, such that cohesin function is reduced as that term is defined herein. STAG2 is the most frequently mutated cohesin subunit in AML (FIG. 2) and is also recurrently mutated in solid tumors. STAG2 is present on the X chromosome, thus mutations in males (or on the active X allele in females) often lead to a complete loss of STAG2 protein, and replacement in the cohesin complex by its paralog STAG1. Importantly, this establishes an absolute dependency of STAG2-mutant cells on expression of STAG1, and both RNAi and CRISPR/Cas9 screens have highlighted STAG1 as a selective, critical dependency in STAG2 mutant cells. Cancer cells that acquire mutations in STAG2 become highly sensitive to loss of the cohesin subunit STAG1, which is a functionally redundant paralog of STAG2 (Viny et al., id., Antony et al., Blood (2018) doi: 10.1182/blood-2018-99-117965, Tothova et al., JCI Insight 6:1-16 (2021), and Jann & Tothova, Blood 138:649-661 (2021). Critically, STAG1 is non-essential in cells with wild-type STAG2 (Viny et al., Cell Stem Cell 25:682-696.e8 (2019)).

As used herein the term “Stromal Antigen 2 (STAG 2) mutant cells” refers to a cell with a genotype that differs from its original genotype due to changes in the DNA of the gene that codes for STAG2. A STAG2 mutant cell will have STAG2 function which is reduced as that term is defined herein, relative to a STAG2 wild-type cell. In some embodiments, a STAG2 mutant cell has less than 50% of the STAG2 expression or function of a wild-type cell, e.g., less than 50%, less than 40%, less than 30%, less than 20%, less than 10%, less than 5%, or no STAG2 expression or function. STAG2 expression can be measured using standard methods. STAG2 function can be measured, e.g., using a splicing reporter construct that depends upon STAG2 for proper splicing.

The inventors have discovered that cohesin mutations common in myeloid malignancies such as myelodysplastic syndromes (MDS) and acute myeloid leukemia (AML) disrupt RNA splicing and render cells highly sensitive to broad-spectrum splicing inhibitors. Through detailed investigation of gene activity and splicing profiles in cohesin-mutant AML cells, several genes were discovered with alternative splicing events that are exacerbated by splicing inhibitors. Remarkably, one of these is a component of the cohesin complex, STAG1. The inventors have identified anti-sense oligonucleotides (ASOs) that selectively modulate splicing and/or stability of STAG1 mRNAs, to precisely target vulnerabilities and induce lethality in cohesin-mutant cells.

STAG1: STAG1 or Stromal Antigen 1 codes for a member of the SCC3 family, which is a component of cohesin, a multisubunit protein complex that provides sister chromatid cohesin along the length of a chromosome from DNA replication through prophase and prometaphase, after which it is dissociated in preparation for segregation during anaphase. STAG1 sequence is known for a number of species, e.g., human STAG1 (NCBI Gene ID: 10274) mRNA (e.g., NM_005862.3) and polypeptide (e.g., NP_005853.2). Antibodies for specific detection of STAG1 are available, for example, from Abcam, see Anti-SA1 antibody [SUSI63B], ab241544. Candidate ASOs for reducing STAG1 expression are presented in Table 1, and include SEQ ID Nos. 1-81. STAG1 has 39 total exons, and NM_005862.3 has 34 exons included.

In some embodiments, the STAG1 nucleic acid includes or is derived from human STAG1 pre-mRNA having the nucleic acid sequence in NC_000003.12 based on the reference GRCh38.p14 Primary Assembly and a range of 136336236 to 136752378 containing 416143 base pairs.

In some embodiments, the STAG1 mRNA sequence includes or is derived from human STAG1 having the following sequence NM_005862.3 (SEQ ID No. 98):

ATTGGCGTGTGGAAAATGCCACCAGATGGCGGGTTAGGATTGCAGCTCCGTTGAAGGCGCGG
CCCCCGCTCCCGAACCCCCGGCGACCACCCCGTAACAACCCCCCCACATCGGGAATAACACA
CCGGAGACTTTTGGGGGGAAACTAGGTCGATGGTCGGCGGCGCCCGGATGGGCAGCTGAGGA
TTGCCTTTGAGGTTATTTTAAAAGTTTTGAGTTGTACAGCACTTGATTATTTTGCTGCATTG
TGAAAGGACCTCTCCAGCAATGATTACTTCAGAATTACCAGTGTTACAGGATTCAACTAATG
AAACTACTGCCCATTCCGATGCTGGCAGCGAGCTTGAAGAAACAGAGGTCAAAGGAAAAAGA
AAAAGGGGTCGTCCTGGCCGGCCTCCATCTACAAATAAGAAACCTCGAAAATCTCCAGGTGA
GAAGAGCAGAATTGAAGCTGGAATTAGAGGAGCAGGCCGTGGAAGAGCTAATGGACACCCTC
AACAGAATGGGGAAGGGGAGCCTGTCACATTATTTGAGGTGGTGAAACTGGGGAAAAGTGCA
ATGCAGTCCGTGGTGGATGACTGGATTGAATCATATAAACAAGACAGGGACATCGCACTTCT
GGATTTAATCAACTTTTTTATCCAGTGTTCAGGATGTCGAGGTACTGTGAGAATAGAGATGT
TTCGAAATATGCAGAATGCAGAAATCATCAGAAAAATGACTGAAGAATTTGATGAGGACAGT
GGTGATTATCCTCTTACCATGCCTGGACCTCAGTGGAAAAAATTTCGTTCAAACTTTTGTGA
ATTTATTGGAGTCCTGATTCGACAGTGTCAGTATAGCATAATTTATGATGAGTATATGATGG
ACACAGTAATCTCCCTTTTGACGGGTTTGTCAGACTCCCAGGTCAGAGCTTTTAGGCATACA
AGTACCCTGGCTGCCATGAAGCTCATGACTGCTCTGGTGAATGTTGCCTTAAACCTCAGTAT
TCATCAGGATAATACCCAGAGACAATATGAAGCCGAGAGAAATAAAATGATTGGGAAGAGAG
CCAATGAAAGGTTGGAGTTACTACTTCAGAAACGCAAAGAGCTGCAAGAAAATCAGGATGAA
ATCGAAAATATGATGAACTCTATTTTTAAGGGTATATTTGTTCATAGATACCGTGATGCTAT
TGCTGAGATTAGAGCCATTTGTATTGAAGAAATTGGAGTATGGATGAAAATGTATAGTGATG
CCTTCCTAAATGACAGTTACCTAAAATATGTTGGCTGGACTCTTCATGACAGGCAAGGGGAA
GTCAGGCTGAAGTGTTTGAAAGCTCTGCAGAGTCTATATACCAATAGAGAATTATTCCCCAA
ATTGGAACTATTCACTAACCGATTCAAGGATCGCATTGTATCAATGACACTTGATAAAGAAT
ATGATGTTGCTGTGGAAGCTATTCGATTGGTTACTCTGATACTTCATGGAAGTGAAGAAGCT
CTTTCCAATGAAGACTGTGAAAATGTTTACCACTTGGTGTACTCGGCACATCGCCCTGTTGC
TGTGGCAGCTGGAGAGTTCCTTCACAAAAAGCTATTTAGCAGACATGACCCACAAGCAGAAG
AAGCATTAGCAAAGAGGAGGGGAAGAAACAGCCCGAATGGAAACCTCATTAGGATGCTGGTT
CTTTTCTTTCTTGAAAGTGAGTTACATGAACATGCAGCCTACTTGGTGGACAGTTTATGGGA
GAGCTCTCAAGAACTGTTGAAAGACTGGGAATGTATGACAGAGTTGCTATTAGAAGAACCTG
TTCAAGGAGAGGAAGCAATGTCTGATCGTCAAGAGAGTGCTCTTATAGAGCTAATGGTTTGT
ACAATTCGTCAAGCTGCTGAGGCACATCCTCCAGTGGGAAGGGGTACCGGCAAGAGAGTGCT
AACTGCCAAAGAAAGGAAAACTCAAATTGATGATAGAAACAAATTGACTGAACATTTTATTA
TTACACTTCCTATGTTACTGTCAAAGTATTCTGCAGATGCAGAGAAGGTAGCAAACTTGCTA
CAAATCCCACAGTATTTTGATTTAGAAATCTACAGCACAGGTAGAATGGAAAAGCATCTGGA
TGCTTTATTAAAACAGATTAAGTTTGTTGTGGAGAAACACGTAGAATCAGATGTTCTAGAAG
CCTGCAGTAAAACCTATAGTATCTTATGCAGTGAAGAATATACCATCCAGAACAGAGTTGAC
ATAGCTCGAAGCCAGCTGATTGATGAGTTTGTAGATCGATTCAATCATTCTGTGGAAGACCT
ATTGCAAGAGGGAGAAGAAGCTGATGATGATGACATTTACAATGTTCTTTCTACATTAAAGC
GGTTAACTTCTTTTCACAATGCACATGATCTCACAAAATGGGATCTCTTTGGTAATTGCTAC
AGATTATTGAAGACTGGAATTGAACATGGAGCCATGCCAGAACAGATAGTCGTGCAAGCACT
GCAGTGTTCCCATTATTCGATTCTTTGGCAGTTGGTGAAAATTACTGATGGCTCTCCTTCCA
AAGAGGATTTGTTGGTATTGAGGAAAACGGTGAAATCCTTTTTGGCTGTTTGCCAGCAGTGC
CTGTCTAATGTTAATACTCCAGTGAAAGAACAGGCTTTCATGTTACTCTGTGATCTTCTGAT
GATTTTCAGCCACCAATTAATGACAGGTGGCAGAGAGGGCCTTCAGCCTTTGGTGTTCAATC
CAGATACTGGACTCCAATCTGAACTCCTCAGTTTTGTGATGGATCACGTTTTTATTGACCAA
GACGAGGAGAACCAGAGCATGGAGGGTGATGAAGAAGATGAAGCTAATAAAATTGAGGCCTT
ACATAAAAGAAGGAATCTACTTGCTGCTTTCAGCAAACTTATCATTTATGACATTGTTGACA
TGCATGCAGCTGCAGACATCTTCAAACACTACATGAAGTATTACAATGACTATGGTGATATT
ATTAAGGAAACACTGAGTAAAACCAGGCAGATTGATAAAATTCAGTGTGCCAAGACTCTCAT
TCTCAGTTTGCAACAGTTATTTAATGAACTTGTTCAAGAGCAAGGTCCCAACCTAGATAGGA
CATCTGCCCATGTCAGTGGCATTAAAGAACTGGCACGTCGCTTTGCCCTTACATTTGGATTG
GACCAGATTAAGACACGAGAAGCAGTTGCCACACTTCACAAGGATGGCATAGAGTTTGCATT
TAAATACCAAAATCAGAAAGGACAAGAGTATCCACCTCCTAATCTGGCTTTTCTTGAAGTAC
TAAGTGAATTTTCTTCTAAACTTCTTCGACAGGACAAAAAGACAGTTCATTCATACCTAGAG
AAATTCCTTACCGAGCAGATGATGGAAAGGAGGGAGGATGTATGGCTTCCACTCATCTCCTA
TAGAAATTCATTAGTCACTGGGGGTGAAGATGATAGAATGTCTGTGAACAGTGGAAGTAGCA
GCAGCAAAACCTCATCAGTAAGGAATAAGAAAGGACGACCTCCACTTCATAAAAAACGAGTA
GAAGATGAGAGTCTGGATAACACATGGCTAAACAGGACTGACACCATGATTCAGACTCCTGG
CCCCCTGCCAGCACCACAACTCACATCCACTGTACTGCGGGAGAACAGTCGGCCCATGGGAG
ACCAGATTCAAGAACCTGAGTCTGAACATGGTTCTGAACCAGACTTTTTACACAATCCTCAG
ATGCAGATCTCTTGGTTAGGCCAGCCGAAGTTAGAAGACTTAAATCGGAAGGACAGAACAGG
AATGAACTACATGAAAGTGAGAACTGGAGTGAGGCATGCTGTTCGGGGTCTAATGGAGGAAG
ATGCTGAGCCCATCTTTGAAGATGTGATGATGTCATCCCGAAGCCAGTTAGAAGATATGAAT
GAAGAATTTGAGGACACCATGGTTATTGATCTGCCTCCATCAAGAAATCGGCGAGAGAGAGC
TGAGCTAAGGCCAGACTTCTTTGACTCTGCAGCTATCATAGAAGATGATTCAGGATTTGGAA
TGCCTATGTTCTGAAGTCTGAAGAAAATTTACAAATCTGGAACTCTATTATTTAGAGCTAGA
GGCCTATATACTGTGATAGCTTGTATGGGGAAAAACACTTTTGATGTGATCTGATTTGTTTT
TTAATCAAATGATTAAGGTCAATCCCTTTTTGCAGTGACAGAAGAGGAGCATGTAAATTACC
CAAGGGAATGTTGGTGAATGTCAACTCAGAAAGACTGACCTGAAAATCATTTGTGTCCTACT
ATTGGACTTATCCCAATACAGATGTGTGTGTTTTTCTGGAGGGAGGAAGAAATTTTAAATTT
TTAAAACAGCTGTCAAGATAAACACTGTTATACACCTGTTTTATGAAAACTCAACATTGAGT
AAAAAAAAACATATTTTTAACTTTATTTTCCTGTTGTACAATTTAAAAACCGTTTTAACATT
TTGCCTTTTTATGTTTTAAAAGCTAACCATTTTTATTAAACCTATGAGTAAGCAGCTCATCC
TAATTGCGAAGAGTGTTTTGGAGTTCACTGGATTTGGTTGACCTTTGTGGAACACAAATAAT
GAAGGAGCAGAACATTGACAAGCTAAGATGAAATTCTGACATAGTACATCTCTGCCAAAAAC
CACACACCCTCTGTGGATATGGATATGAATTCCCAGATTTTATATACTCTTGAATAAAAGGT
TTATTTTTATTTATAAGTGGGCATAAAATAAGAAATGTCCATGCAGCCATTTTTCCAACAGA
TGCTGTACACCGTTCATTTTATATAGACTAGGGAGATTCAAATACAGTGCATTTTCTATTGG
TATTTGTTCTGTGCATTTTTAGCAACTTCTACCAGCAAATAAAGTATTCTCAGTAAAACGAA
AATGATTCTCAAGTTATCAGTTTGCTGTTTTTACCACTTATTTCATGCCCTGCCAAATTCAA
GTTACACAGACTTCCATTTTCTTAAGATAATCAATCATGAAGAAATCCTTTATCAATCATTC
AAAAGTAATTTTAAGTGTAACATAACTGTGTTTACTTCCCATGCACTTAATACCCTTATGCG
CTAATTTTGTGAATTAAGTTTACTGATTATAGAAGTATGTGCTGCATAGAAGTCTGTGCTTA
GAGGGTGAAGTTCCTAAGCTTACCTTGAATTACAGCTACATTTCAGTGTTAAATGTGCATAT
TAAGAATAATTCTTTTGGGGAAAGAAATTATGAATCTTCAGGACAGTCTACAATGGTTTAGA
GTTACATTCTGCCTAGACTTTTATGACTTGCTGCTATTGTTTTAAAAACCCCACTTAGTCTC
TCTCTTCCTTTCTGATTTCTAAAGTAAGCCTCAGAATTTCCAAACCAATTCATCCACAGCTG
TTTCTGGGCTGGTTTTTAAAGTAGCTGCAACAGAATCATGAGGCTTTCCCTTTTTATCAAAT
ACGAAAAACATTTTTTAAAATTCTGCACACCCAGTGATCATCTTTTGTGCGGGAAAGCAAGA
TGATGATGGATGATTTTATTCATCCTTTTAGTAAAGACACAAAACATTTTTCTCAACATTTG
TACAGTTCTGAAAAAAACCTGGTCACCAAAAATATCTTCTCTGCTAATTCAGCAATTCTTGG
GCTCCAGTTAGGGGAGCTGGGGCCTCACTTTCTCCCAGAATTGTGGGCTTCACTGGAAGTGA
AGGTGCAGGAATGACTGGACTGTCCACCCCAGCCCTGCCTGCCTGTGGTTTTGGCCAGGGAG
CAAGCCATGAGGTGCCCTGGCACATGCACAAATTGATCCTTTGCGTGACAGTCTTGTATGGA
AAACAGATGCTGACAGAATTGTAGACTACCATGCCACACAAAAAGGCTAAATATCTACTCCA
ATGGGTTTCCAGTTCAGTTTGAAGTCAATCAAATTTTTGTATTTTCGGTGTCTCCTTGATTG
GTTTTGCTAGTAATTCTGTAAATTGTACATTTGCAATATGAGGTTTTTTTTCCTTTTGTACA
ATTTGAAACTGATGCTTCACCTTTCCTTTAATAAACTATTCAAAATCA

The locations of the 34 exons that are joined together to form this transcript by reference of the STAG1 genomic sequence NC_000003.12 are the following:
Exon 1: 1 . . . 184; Exon 2: 121398 . . . 121509; Exon 3:
129131 . . . 129233; Exon 4: 147906 . . . 148070; Exon 5:
183518 . . . 183614; Exon 6: 210184 . . . 210260; Exon 7:
230962 . . . 231166; Exon 8: 249600 . . . 249751; Exon 9:
252083 . . . 252156; Exon 10: 274967 . . . 275090; Exon 11:
278742 . . . 278840; Exon 12: 279887 . . . 279966; Exon 13:
287391 . . . 287498; Exon 14: 300232 . . . 300346; Exon 15:
308975 . . . 309092; Exon 16: 318720 . . . 318823; Exon 17:
329335 . . . 329427; Exon 18: 329522 . . . 329611; Exon 19:
329766 . . . 329969; Exon 20: 331216 . . . 331286; Exon 21:
334407 . . . 334494; Exon 22: 353550 . . . 353630; Exon 23:
374627 . . . 374719; Exon 24: 383097 . . . 383271; Exon 25:
385297 . . . 385436; Exon 26: 388912 . . . 389013; Exon 27:
393083 . . . 393231; Exon 28: 394531 . . . 394659; Exon 29:
403016 . . . 403221; Exon 30: 408373 . . . 408547; Exon 31:
410828 . . . 410938; Exon 32: 411774 . . . 411888; Exon 33:
413929 . . . 414009; Exon 34: 414102 . . . 416143

In some embodiments, the STAG1 polypeptide includes or is derived from human STAG1 having the following amino acid sequence NP_005853.2 (SEQ ID No. 82):

MITSELPVLQDSTNETTAHSDAGSELEETEVKGKRKRGRPGRPPSTNKKP
RKSPGEKSRIEAGIRGAGRGRANGHPQQNGEGEPVTLFEVVKLGKSAMQS
VVDDWIESYKQDRDIALLDLINFFIQCSGCRGTVRIEMFRNMQNAEIIRK
MTEEFDEDSGDYPLTMPGPQWKKFRSNFCEFIGVLIRQCQYSIIYDEYMM
DTVISLLTGLSDSQVRAFRHTSTLAAMKLMTALVNVALNLSIHQDNTQRQ
YEAERNKMIGKRANERLELLLQKRKELQENQDEIENMMNSIFKGIFVHRY
RDAIAEIRAICIEEIGVWMKMYSDAFLNDSYLKYVGWTLHDRQGEVRLKC
LKALQSLYTNRELFPKLELFTNRFKDRIVSMTLDKEYDVAVEAIRLVTLI
LHGSEEALSNEDCENVYHLVYSAHRPVAVAAGEFLHKKLFSRHDPQAEEA
LAKRRGRNSPNGNLIRMLVLFFLESELHEHAAYLVDSLWESSQELLKDWE
CMTELLLEEPVQGEEAMSDRQESALIELMVCTIRQAAEAHPPVGRGTGKR
VLTAKERKTQIDDRNKLTEHFIITLPMLLSKYSADAEKVANLLQIPQYFD
LEIYSTGRMEKHLDALLKQIKFVVEKHVESDVLEACSKTYSILCSEEYTI
QNRVDIARSQLIDEFVDRFNHSVEDLLQEGEEADDDDIYNVLSTLKRLTS
FHNAHDLTKWDLFGNCYRLLKTGIEHGAMPEQIVVQALQCSHYSILWQLV
KITDGSPSKEDLLVLRKTVKSFLAVCQQCLSNVNTPVKEQAFMLLCDLLM
IFSHQLMTGGREGLQPLVFNPDTGLQSELLSFVMDHVFIDQDEENQSMEG
DEEDEANKIEALHKRRNLLAAFSKLIIYDIVDMHAAADIFKHYMKYYNDY
GDIIKETLSKTRQIDKIQCAKTLILSLQQLFNELVQEQGPNLDRTSAHVS
GIKELARRFALTFGLDQIKTREAVATLHKDGIEFAFKYQNQKGQEYPPPN
LAFLEVLSEFSSKLLRQDKKTVHSYLEKFLTEQMMERREDVWLPLISYRN
SLVTGGEDDRMSVNSGSSSSKTSSVRNKKGRPPLHKKRVEDESLDNTWLN
RTDTMIQTPGPLPAPQLTSTVLRENSRPMGDQIQEPESEHGSEPDFLHNP
QMQISWLGQPKLEDLNRKDRTGMNYMKVRTGVRHAVRGLMEEDAEPIFED
VMMSSRSQLEDMNEEFEDTMVIDLPPSRNRRERAELRPDFFDSAAIIEDD
SGFGMPMF

STAG2: STAG2 or Stromal Antigen 2 codes for a subunit of the cohesin complex, which regulates the separation of sister chromatids during cell division. Targeted inactivation of this gene results in chromatid cohesin defects and aneuploidy, suggesting that genetic disruption of cohesin is a cause of aneuploidy in human cancer. Alternatively spliced transcript variants encoding different isoforms have been found for this gene STAG2 sequence is known for a number of species, e.g., human STAG2 (NCBI Gene ID: 10735) mRNA (e.g., NM_001042749.2) and polypeptide (e.g., NP_001036214.1). Antibodies for specific detection of STAG2 are available, for example, from Abcam, see Anti-SA2 antibody [EPR17865], ab201451. STAG2 has 40 total exons, and NM_001042749.2 has 35 exons included.

In some embodiments, the STAG2 nucleic acid includes or is derived from human STAG2 pre-mRNA having the nucleic acid sequence in NG_033796.2 based on the reference RefSeqGene (LRG_782) on chromosome X and a range of 5001 to 147097 containing 142097 base pairs.

In some embodiments, the STAG2 mRNA sequences includes or is derived from human STAG2 having the following sequence NM_001042749.2 (SEQ ID No. 83):

GTCGCCGAAGAGCGAACACCCCAAACAATCCCGAAGCGCCACCAAAAAAAAAAAAAAAAAAA
AAAAAAAAAAAAAAAAAAAAAAGAAAAAAAACCCCGCCGGATCCGACCGCCACTTTCAAAAC
CCCCCACCGCTCTAGAACCGCGGGAGCTTCCGTCCCTGAGTAGAATTCGAGGGTGTAAAGAA
GAGGAAGGGGAAAAATATCTTGTACCAGCCCAGGGGTGAAGAAGCCCCCGGCCTGAGAAAGA
AGGAGGAGTGGGGGAGGCGAACAGTCTCGTTGCTGCCTCTGTGTACGCTGAGGGGGGAGGTG
GCCACCGAGTACTAAATTCACTTGGGAATAAAAGAAAAACATAAGAAAATTATAAGAGAAAG
GAATTGTCTTAGAAGAAAGAAGGCAAGCCACCATTTTACCCACGTAAATATATGAATATATT
TCTGACATTGAGGTGTTCCAGAAGATGATAAAGAAATGATAGCAGCTCCAGAAATACCAACT
GATTTTAATCTACTACAGGAGTCAGAAACACATTTTTCTTCTGACACAGATTTTGAAGATAT
CGAAGGAAAAAACCAAAAGCAAGGCAAAGGCAAAACTTGTAAAAAAGGCAAAAAGGGCCCAG
CAGAAAAGGGCAAAGGTGGAAATGGAGGAGGAAAACCTCCTTCTGGTCCAAACCGAATGAAT
GGTCATCACCAACAGAATGGAGTGGAAAACATGATGTTGTTTGAAGTTGTTAAAATGGGCAA
GAGTGCTATGCAGTCGGTGGTAGATGATTGGATAGAATCATACAAGCATGACCGAGATATAG
CACTTCTTGACCTTATCAACTTTTTTATTCAGTGTTCAGGCTGTAAAGGAGTTGTCACAGCA
GAAATGTTTAGACATATGCAGAACTCTGAGATAATTCGAAAAATGACTGAAGAATTCGATGA
GGATAGTGGAGATTATCCACTTACCATGGCTGGTCCTCAGTGGAAGAAGTTCAAATCCAGTT
TTTGTGAATTCATTGGCGTGTTAGTACGGCAATGTCAATATAGTATCATATATGATGAGTAT
ATGATGGATACAGTCATTTCACTTCTTACAGGATTGTCTGACTCACAAGTCAGAGCATTTCG
ACATACAAGCACCCTGGCAGCTATGAAGTTGATGACAGCTTTGGTGAATGTGGCACTAAATC
TTAGCATTAATATGGATAATACACAAAGACAATATGAAGCAGAACGGAATAAAATGATTGGA
AAACGAGCCAATGAGAGGCTAGAACTCCTGCTACAAAAGCGGAAAGAGCTTCAGGAAAATCA
AGATGAAATAGAAAATATGATGAATGCAATATTTAAAGGAGTGTTTGTACATAGATACCGTG
ATGCGATAGCTGAAATTCGAGCTATTTGCATTGAAGAGATTGGCATTTGGATGAAGATGTAT
AGTGATGCCTTTCTTAATGACAGTTATTTAAAATATGTTGGTTGGACTATGCATGATAAGCA
AGGTGAAGTAAGACTCAAATGTCTTACTGCTCTACAAGGGCTTTATTATAACAAAGAGCTTA
ATTCCAAACTGGAACTTTTTACCAGTCGGTTCAAGGATAGAATTGTGTCTATGACCCTTGAC
AAAGAATATGATGTTGCAGTACAAGCAATAAAATTACTCACTCTTGTTTTACAGAGTAGTGA
AGAAGTTCTCACTGCAGAAGATTGTGAAAATGTCTATCATCTGGTTTATTCAGCTCACCGGC
CAGTAGCAGTAGCAGCTGGAGAATTTCTCTACAAAAAGCTCTTCAGTCGTAGAGATCCAGAG
GAGGATGGAATGATGAAAAGAAGAGGAAGACAAGGTCCAAATGCCAACCTTGTTAAGACATT
GGTTTTTTTCTTTCTAGAAAGTGAGTTACATGAGCATGCAGCATACCTTGTGGATAGCATGT
GGGACTGTGCTACTGAGCTGCTGAAAGACTGGGAATGTATGAATAGCTTGTTACTGGAAGAG
CCACTTAGTGGAGAGGAAGCACTAACAGATAGGCAAGAGAGTGCTCTGATTGAAATAATGCT
TTGTACCATTAGACAAGCGGCTGAATGTCATCCTCCCGTGGGAAGAGGGACAGGAAAAAGGG
TGCTTACAGCAAAGGAGAAGAAGACACAGTTGGATGATAGGACAAAAATCACTGAGCTTTTT
GCCGTGGCCCTTCCTCAGTTATTAGCAAAATACTCTGTAGATGCAGAAAAGGTGACTAACTT
GTTGCAGTTGCCTCAGTACTTTGATTTGGAAATATATACCACTGGACGATTAGAAAAGCATT
TGGATGCCTTATTGCGACAGATCCGGAATATTGTAGAGAAGCACACAGATACAGATGTTTTG
GAAGCATGTTCTAAAACTTACCATGCACTCTGTAATGAAGAGTTCACAATCTTCAACAGAGT
AGATATTTCAAGAAGTCAACTGATAGATGAATTGGCAGATAAATTTAACCGGCTTCTTGAAG
ATTTTCTGCAAGAGGGTGAAGAACCTGATGAAGATGATGCATATCAGGTATTGTCAACATTG
AAGAGGATCACTGCTTTTCATAATGCCCATGACCTTTCAAAGTGGGATTTATTTGCTTGTAA
TTACAAACTCTTGAAAACTGGAATCGAAAATGGAGACATGCCTGAGCAGATTGTTATTCACG
CACTGCAGTGTACTCACTATGTAATCCTTTGGCAACTTGCTAAGATAACTGAAAGCAGCTCT
ACAAAGGAGGACTTGCTGCGTTTAAAGAAACAAATGAGAGTATTTTGTCAGATATGTCAACA
TTACCTGACCAACGTGAATACTACTGTTAAGGAACAGGCCTTCACTATTCTGTGTGATATTT
TGATGATCTTCAGCCATCAGATTATGTCAGGAGGGCGTGACATGTTAGAGCCATTAGTGTAT
ACCCCTGATTCTTCATTGCAGTCTGAGTTGCTCAGCTTTATTTTGGATCATGTCTTCATTGA
ACAGGATGATGATAATAATAGTGCAGATGGTCAGCAAGAGGATGAAGCCAGTAAAATTGAAG
CTCTGCACAAGAGAAGAAATTTACTTGCAGCATTTTGTAAGCTAATTGTATATACTGTGGTG
GAGATGAATACAGCTGCAGATATCTTCAAACAGTATATGAAGTATTATAATGACTATGGAGA
TATCATCAAAGAAACAATGAGTAAAACAAGGCAGATAGACAAAATTCAGTGTGCTAAGACCC
TTATTCTCAGTCTGCAACAGCTTTTTAATGAAATGATACAAGAAAATGGCTATAATTTTGAT
AGATCATCCTCTACATTTAGTGGCATAAAAGAACTTGCTCGACGTTTTGCTTTAACTTTTGG
ACTTGATCAGTTGAAAACAAGAGAAGCCATTGCCATGCTACACAAAGATGGCATAGAATTTG
CTTTTAAAGAGCCTAATCCGCAAGGGGAGAGCCATCCACCTTTAAATTTGGCATTTCTTGAT
ATTCTGAGTGAATTTTCTTCTAAACTACTTCGACAAGACAAAAGAACAGTGTATGTTTACTT
GGAAAAGTTCATGACCTTTCAGATGTCACTCCGAAGAGAGGATGTGTGGCTTCCACTGATGT
CTTACCGAAATTCTTTGCTAGCTGGTGGTGATGATGACACCATGTCAGTCATTAGTGGAATC
AGCAGCCGGGGGTCAACAGTACGGAGTAAAAAATCAAAACCATCTACAGGAAAACGGAAAGT
GGTTGAGGGCATGCAGCTTTCACTCACTGAAGAAAGTAGTAGTAGTGACAGTATGTGGTTAA
GCAGAGAACAAACACTGCACACCCCTGTTATGATGCAGACACCACAACTCACCTCCACTATT
ATGAGAGAGCCCAAAAGATTACGGCCTGAGGATAGCTTCATGAGTGTTTATCCAATGCAGAC
TGAACATCATCAAACACCTCTTGATTATAACACGCAGGTAACATGGATGTTAGCTCAAAGAC
AACAAGAGGAAGCAAGGCAACAGCAGGAGAGAGCAGCAATGAGCTATGTTAAACTGCGAACT
AATCTTCAGCATGCCATTCGGCGTGGCACAAGCCTAATGGAAGATGATGAAGAGCCAATTGT
GGAAGATGTTATGATGTCCTCAGAAGGGAGGATTGAGGATCTTAATGAGGGAATGGATTTTG
ACACCATGGATATAGATTTGCCACCATCAAAGAACAGACGAGAGAGAACAGAACTGAAGCCT
GATTTCTTTGATCCAGCTTCAATTATGGATGAATCAGTTCTTGGAGTGTCAATGTTTTAATA
CCAGTACACAATTAAATCTGTGGTGAAGTCATTTTCTAAGTGGAAGAGGAAATTTTAAAGTG
TGGTAGATACAGTGAAATTCTGTACAGATTTTTCTCTAAGGAGAATATGACATGCTTATGCT
TACCAAGATCAAGTGCATTGAGGGGCAGTTTTGTTTGCCTGAATAAACGTAAAGGACAAGTA
AACAATTTGATGATAAGCTACAGTTTTTCTTAGAAAGTAAATATTTTATTTATGCGCTGTTA
GTTGGCTTTTGAATCGATTATTTCATGCTTTTTTTTAAAAAAAAAAAAAAACAAAATAACAA
TCTGAAGAGGCATTTGGTACAGATATGAATTCTCTTACATTTATTTACTGGTTGTACTAAAT
AATGATGACCTCTGCTGGATTTCTGTTTACATCCAGAAAACAATGTTAAGGATGTATTTATT
CCCCTACCCTGAAGAAAGTGTAGGATAGAATTGTTTTTAGCATTCTAAATTTAAATGCTTAA
AACGTCAATCAACAAAACTTTGTTTTAAATATTGTAATTGTGGAGAAAAGTAAACTTATAAG
CAGAACTTTTACAATTTTTTCATCTAAAAGTATTTTAAGATATTTTTAAAATCCAAGAGCTT
CTCTATACTTTTCAGAAATATCCAGATGCAGTGAACTGCCAGAAGGTAACCAGTCTCAAACA
TGCTTATCCCATTATCAACCCTGAAAGTTTGCTTGTCCTTTAAGATAAAAATGTAATGTTGT
GATATTCCTTCCAGTAATGCCACTGTATTTTGTCTCCAAATAAAAGAAGCTTATTGTAGTAT
GTTTGCAGAAAAATTCTAAACAAAAATTATACAGCTTATTAGAGTGTGGGAATAGGGATCTA
AATTTTAAATAAAATTATATATATATATAAATTGGTGCTGATTTTATAATTGCGCAGTTTGT
TTAGTTTTTTCTTACTTTTAAATTCCAACTTAAAATTATGAGGTTTCAGAAATATATTGAAA
GTTTAACAATGTTTAAAAATAGAAAAGCATGAGTGTTCATGCTTTAAAATGATTTTTAAATT
TGTATTTTATATTGTTTTATCTATCTGTCTTTGCAAGCAGTCTTCAGGTTAAAGATACTTCT
AACAGGTTACAGTACATTTCCTCTGTATGTAAATTAGATGGGATAATAGAATTCATAACCCA
TAATATTCTTTGAAAGCTAAGCTTTAAACTTCATTTTATGTCCTTTCACAAATAAATTAGTT
TAAAACAGAAAGTGGCTACTTGCCATTTTGACATCAACTCATTTTGCGAGGCTTAGGCAGCT
AGACATCGTTTAAAACAAAATATTAACTTATATTACATGTGTATCTATCTATTGTCAGTCGT
CTCTCAGTTCTTGAGGTATATTATTTTAATCATTCCATGCCTTAATATGCTTGCAATACAAG
AATATCTTCAGATGGGTGAATACCAAAAGGCTTTCAGTTTTTAGTCAGAAATCAAGCATTGG
GCTGTGGTAGCCAAAAACCATAGGTTAGCTAAAAAGATCATGATACAATTATTTTATTAAGT
CATGGTTAATAACAAATGAATCCAGACTTGTCTAACAGATTTTCCATCAACAAATATTGTTA
TGTGCAAAAGTATTGCCTATGTTGTTTTACACACCACTGCATTAACTAGAACTGCTGAGAGG
ACTGTATATATGATTTTAAACCTAAGTTGATTTTTTTTCTCACTCTTGAAAGGAGTACTTCT
TTGTGAAAGCAGTTCTTACAGCTTTGTTTTCAACCAGCTAAAAATGTTTTATATATTACTCT
AACCTGTTGTCCTCCACATTCTATTGTCCTAATTGTACTGTTTTCTGATTTGTATTTATGTC
TTGAGACAGTAACTTTTTGAATAAAAATAAACCTACAGTATGTTGTATGTTTTCTCTTGTAC
TCAAAGGGGGAGGGTGGCTATAAATGGTTTGCAAATTTATATCTATTATCACATCTTTTAAT
GTGTTTGGGGAATAATTTATAGAGAATACCATCAGTTTATATTTTTAATAAATCATATGTAT
TTACAATGAAAAAAAAAA

In some embodiments, the STAG2 polypeptide includes or is derived from human STAG2 having the following amino acid sequence NP_001036214.1 (SEQ ID No. 99):

MIAAPEIPTDFNLLQESETHFSSDTDFEDIEGKNQKQGKGKTCKKGKKGP
AEKGKGGNGGGKPPSGPNRMNGHHQQNGVENMMLFEVVKMGKSAMQSVVD
DWIESYKHDRDIALLDLINFFIQCSGCKGVVTAEMFRHMQNSEIIRKMTE
EFDEDSGDYPLTMAGPQWKKFKSSFCEFIGVLVRQCQYSIIYDEYMMDTV
ISLLTGLSDSQVRAFRHTSTLAAMKLMTALVNVALNLSINMDNTQRQYEA
ERNKMIGKRANERLELLLQKRKELQENQDEIENMMNAIFKGVFVHRYRDA
IAEIRAICIEEIGIWMKMYSDAFLNDSYLKYVGWTMHDKQGEVRLKCLTA
LQGLYYNKELNSKLELFTSRFKDRIVSMTLDKEYDVAVQAIKLLTLVLQS
SEEVLTAEDCENVYHLVYSAHRPVAVAAGEFLYKKLFSRRDPEEDGMMKR
RGRQGPNANLVKTLVFFFLESELHEHAAYLVDSMWDCATELLKDWECMNS
LLLEEPLSGEEALTDRQESALIEIMLCTIRQAAECHPPVGRGTGKRVLTA
KEKKTQLDDRTKITELFAVALPQLLAKYSVDAEKVINLLQLPQYFDLEIY
TTGRLEKHLDALLRQIRNIVEKHTDTDVLEACSKTYHALCNEEFTIFNRV
DISRSQLIDELADKFNRLLEDFLQEGEEPDEDDAYQVLSTLKRITAFHNA
HDLSKWDLFACNYKLLKTGIENGDMPEQIVIHALQCTHYVILWQLAKITE
SSSTKEDLLRLKKQMRVFCQICQHYLTNVNTTVKEQAFTILCDILMIFSH
QIMSGGRDMLEPLVYTPDSSLQSELLSFILDHVFIEQDDDNNSADGQQED
EASKIEALHKRRNLLAAFCKLIVYTVVEMNTAADIFKQYMKYYNDYGDII
KETMSKTRQIDKIQCAKTLILSLQQLFNEMIQENGYNFDRSSSTFSGIKE
LARRFALTFGLDQLKTREAIAMLHKDGIEFAFKEPNPQGESHPPLNLAFL
DILSEFSSKLLRQDKRTVYVYLEKFMTFQMSLRREDVWLPLMSYRNSLLA
GGDDDTMSVISGISSRGSTVRSKKSKPSTGKRKVVEGMQLSLTEESSSSD
SMWLSREQTLHTPVMMQTPQLTSTIMREPKRLRPEDSFMSVYPMQTEHHQ
TPLDYNTQVTWMLAQRQQEEARQQQERAAMSYVKLRTNLQHAIRRGTSLM
EDDEEPIVEDVMMSSEGRIEDLNEGMDFDTMDIDLPPSKNRRERTELKPD
FFDPASIMDESVLGVSMF

A STAG2 mutant cell includes a cell in which the STAG2 gene is mutated relative to the wild-type STAG2 gene such that the expression or activity of STAG2 is lost or substantially deficient (e.g., reduced by 80% or more, 90% or more, 95% or more, 96% or more, 97% or more, 98% or more, 99% or more, or 100% relative to wild-type). Loss of STAG2 renders cells dependent upon STAG1 for survival, rendering disease involving STAG2-deficient cells amenable to treatment with STAG1 inhibitors.

In such embodiments, early diagnosis of STAG2-deficient MDS and treatment with antisense oligonucleotides targeting STAG1 as described herein can provide benefits relative to treating commenced when the disease has progressed to AML.

In the methods described herein, inhibition of STAG1 expression is effective for selectively killing cells that are deficient in STAG2, which is frequently mutated in various cancers, as discussed herein above. STAG2 expression can be examined by measurement of (spliced) RNA levels, e.g., via RT-PCR using primers that span one or more introns. The level of STAG2 expression can also be determined, e.g., by protein assay, e.g., a STAG2 immunoassay as known to those of skill in the art (including, but not limited to Western blot, ELISA, immunoprecipitation, etc.), mass spectrometry, or other methods known to those of skill in the art. Levels of STAG2 RNA or protein in cancer cells can be compared to an appropriate control, e.g., the level in normal cells of the same or similar lineage. A lack of, or alternatively a reduction in STAG2 expression by at least 70%, 80%, 90% or more relative to control is considered STAG2 deficient as the term is used herein. Whether a patient's cancer has a STAG2 mutation or disruption can also be determined by clinical exome sequencing, i.e., targeted RNA sequencing of RNA from the patient's cancer cells. Changes in amino acid coding sequence, whether frameshifts or other mis-sense mutations or amino acid altrations, mutations that interfere with or alter splice sites, or that introduce premature stop codons, among others, can indicate that a given cancer cell is STAG2 deficient.

### Antisense Oligonucleotides

As discussed herein, target gene expression can be reduced by administering antisense oligonucleotides complementary to selected region(s) of the target transcript. Antisense oligonucleotides that target splicing of the STAG1 transcript are of particular interest. Interference with splicing can reduce the amount of correctly- or fully-spliced mRNA encoding STAG1 and thereby reduce STAG1 protein expression. Non-limiting examples of ASOs that can interfere with splicing include those that hybridize to or overlap 5′ splice sites, and those that hybridize to or overlap exonic splicing enhancer elements, in the primary transcript. In some embodiments, ASO overlap with exonic splicing enhancers on the STAG1 RNA transcript is by at least one nucleotide, but preferably overlap is by 2, 3, 4, 5, 6, 7, 8 or more nucleotides. In some embodiments, the ASO compositions as described herein target the RNA for degradation, e.g., via RNAse H-mediated degradation. Non-limiting examples include gapmers as described herein, which comprise RNA-DNA-RNA chimeric oligos which tend to promote RNAse H activity against the hybridized target RNA. In some embodiments, the ASOs as described herein target elements that influence RNA splicing and target the RNA transcripts for degradation. Such a combined approach can provide improvements in knockdown of target gene expression. As such, in another embodiment of this and any other aspect described herein, hybridization of the antisense oligonucleotide overlaps a 5′ splice site or an exonic splicing enhancer (ESE) on the STAG1 RNA transcript. ESEs occur in both alternative and constitutive exons, where they provide binding sites for Ser/Arg-rich proteins (SR proteins), a family of conserved splicing factors that participate in a number of steps of the splicing pathway. Different SR proteins have different substrate specificities, and numerous classes of ESE consensus motifs have been described (see, e.g., Blencowe, Trends Biochem. Sci. 25:106-110 (2000); Cartegni et al., Nat. Rev. Genet. 3:285-298, Graveley, RNA 6:1197-1211 (2000), and Fairbrother et al., Science 297:1007-1013 (2002). A web resource for identifying exonic splicing enhancers is described by Cartegni et al., Nucl. Acids res. 31:3568-3571 (2003).

In some embodiments, the exonic splicing enhancer comprises a binding site for RNA binding protein SRSF2. As used herein the term “binding site for RNA binding protein SRSF2” refers to an RNA sequence to which splicing regulator serine-arginine (SR) protein 2 (SRSF2) can bind. SRSF2 is an SR protein that recognizes and binds to certain ESEs. SRSF2 encodes a 221 amino acid protein that represents the only nuclear-retained member of the SR protein family (14, 15). It contains two functional domains that include an RNA-binding motif and serine-arginine rich domain that is heavily phosphorylated. Proline 95 (P95) lies in a linker region between the RNA binding motif and SR rich region (16). SRSF2 binds to cis elements on pre-mRNA transcripts that functionally redefine putative exon-intron boundaries. Sequences recognized and bound by SRSF2 have been examined using SELEX; see, e.g., Tacke & Manley, EMBO J. 14:3540-3551 (1995), and Lui et al., Mol. Cell. Biol. 20:1063-1071 (2000). These publications also describe SRSF2-binding assays. Considering RNA sequences identified in this manner with approaches for identifying ESEs known in the art or discussed herein can permit the determination of whether a given SRSF2 binding sequence in an RNA would likely be involved in splicing, and in vitro assays for SRSF2 binding can be carried out to confirm whether SRSF2 binds a given RNA sequence.

As used herein, an “antisense oligonucleotide” is a synthetic single-stranded nucleic acid molecule that is complementary to a sequence on an RNA transcript, such as that of a 5′ splice site, exon, intron, protein-binding motif or other transcript sequence element. Oligonucleotides are chosen that are sufficiently complementary to the target (as that term is defined herein), to give the desired effect. For example, an antisense oligonucleotide can comprise at least 8, at least 10, at least 15, at least 20, at least 25, at least 30, at least 35 or more bases complementary to a portion of a STAG1 transcript. Non-limiting examples are provided in Table 1.

As used herein, the term “RNA oligonucleotide” refers to polymers of nucleosides that include the sugar ribose as a component and that are covalently bonded, typically by phosphodiester linkages between subunits, but in some cases for modified RNAs by phosphorothioates, methylphosphonates, and the like.

As used herein, the term “DNA oligonucleotide” refers to polymers of nucleosides that include the sugar deoxyribose as a component and that are covalently bonded, typically by phosphodiester linkages between subunits, but in some cases for modified DNAs by phosphorothioates, methylphosphonates, and the like.

As used herein, the term “sequence sufficiently complementary to a portion of a STAG1 primary transcript to permit hybridization” refers to a nucleic acid sequence, e.g., an antisense oligonucleotide sequence, that is, at least in part, complementary to a target sequence, e.g., STAG1 and has enough complementarity to form a duplex through Watson-Crick base pairing under physiological conditions with the target sequence. The degree of complementarity needed for a given nucleic acid to hybridize or form a duplex with another under physiological conditions depends upon the length and specific nucleotide makeup (e.g., % GC vs % AT or AU content) of the nucleic acid. A calculation of the free energy of binding of a nucleic acid with its complement or with a molecule with at least partial complementarity can provide a prediction of whether a given sequence will hybridize to another under given conditions. Determination of binding free energies for nucleic acid molecules is well known in the art (see, e.g., Turner et al, 1987, CSH Symp. Quant. Biol. LII pp. 123-133; Frier et al., 1986, Proc. Nat. Acad. Sci. USA 83:9373-9377; Turner et al., 1987, I. Am. Chem. Soc. 109:3783-3785). Furthermore, calculations and/or predictions of hybridization energy can be determined using software tools or modeling known in the art, including but not limited to S-Fold, available on the world wide web at sfold.wadsworth.org; PFRED, available on the world wide web at ncbi.nlm.nih.gov/pms/articles/PMC7822268; OligoEvaluator from Sigma available on the world wide web at “oligoevaluator.com/oligocalcservlet; OligoAnalyzer from IDT available on the world wide web at idtdna.com/pages/tools/oligoanalyzer; see also, e.g., Wang et al., 2022, Plos One. 17(5), and Tulpan et al., 2010 BMC Bioinformatics 105.

In some embodiments, the binding or hybridization of an antisense oligonucleotide is capable of halting expression of the target at the level of transcription, translation, or splicing. These sequences hybridize sufficiently well and with sufficient specificity in the context of the cellular environment to give the desired effect. An oligonucleotide sequence that is sufficiently complementary to a target sequence can be complementary over 85%, 90%, 95% or more of the sequence that corresponds to the target sequence. In some embodiments, a sequence that is sufficiently complementary as the term is used herein sequence contains no more than 1, 2, 3, or 4 mismatched nucleotides that are not complementary to the target sequence. In another embodiment, the sequence is 100% complementary to the target sequence.

In some embodiments of any of the aspects, the antisense oligomer consists of from 8 to 40 nucleobases. In some embodiments of any of the aspects, the antisense oligomer consists of from 8 to 40 nucleobases, 8 to 35 nucleobases, 8 to 30 nucleobases, 8 to 25 nucleobases, 8 to 20 nucleobases, 8 to 15 nucleobases, 9 to 40 nucleobases, 9 to 35 nucleobases, 9 to 30 nucleobases, 9 to 25 nucleobases, 9 to 20 nucleobases, 9 to 15 nucleobases, 10 to 40 nucleobases, 10 to 35 nucleobases, 10 to 30 nucleobases, 10 to 25 nucleobases, 10 to 20 nucleobases, 10 to 15 nucleobases, 11 to 40 nucleobases, 11 to 35 nucleobases, 11 to 30 nucleobases, 11 to 25 nucleobases, 11 to 20 nucleobases, 11 to 15 nucleobases, 12 to 40 nucleobases, 12 to 35 nucleobases, 12 to 30 nucleobases, 12 to 25 nucleobases, 12 to 20 nucleobases, or 12 to 15 nucleobases.

In some embodiments of any of the aspects, the antisense oligomer is at least 80%, at least 85%, at least 90%, at least 95%, at least 98%, at least 99%, or 100% identical to a sequence selected from SEQ ID Nos 1-81.

As used herein, the term “nucleotide” refers to an organic molecule that serves as the monomer unit for forming the nucleic acid polymers deoxyribonucleic acid (DNA) and ribonucleic acid (RNA). Conventional, naturally-occurring nucleotides are the building blocks of nucleic acids and are composed of three subunit molecules: a nitrogenous base, a five-carbon sugar, and at least one phosphate group. A nucleoside has a nitrogenous base and a five-carbon carbohydrate, a ribose for a ribonucleoside or a deoxyribose for a deoxynucleoside. In standard nomenclature, addition of one or more phosphate groups to a nucleoside results in a nucleotide. Nucleotides can be modified. Modifications to nucleotides or oligonucleotides comprised of them can improve, for example, stability, strength of hybridization, and/or cellular delivery or uptake characteristics. Tolerable modifications maintain the ability to hybridize to sequence in an RNA transcript and interfere with protein expression.

It should be understood that antisense oligonucleotides applicable in the compositions and methods described herein can include oligos that are 100% DNA, 100% RNA, any combination of ribonucleosides (RNA) and deoxyribonucleosides (DNA), unmodified in regard to sugar, nucleobase and phosphate backbone, as well as oligos that are modified with regard to sugar, nucleobase and/or backbone linkages.

It is often beneficial to include chemical modifications in oligonucleotides to alter their activity. Chemical modifications can alter oligonucleotide activity by, for example: increasing affinity of an antisense oligonucleotide for its target RNA, increasing nuclease resistance, and/or altering the pharmacokinetics of the oligonucleotide. The use of chemistries that increase the affinity of an oligonucleotide for its target can allow for the use of shorter oligonucleotides. Antisense oligonucleotides as described herein can also contain one or more nucleosides having modified sugar moieties. The furanosyl sugar ring of a nucleoside can be modified in a number of ways including, but not limited to, addition of a substituent group, bridging of two non-geminal ring atoms to form a bicyclic nucleic acid (BNA) and substitution of an atom or group such as —S—, —N(R)— or -C(R1)(R2) for the ring oxygen at the 4′-position. Modified sugar moieties are well known and can be used to alter, typically increase, the affinity of the antisense oligonucleotide for its target and/or increase nuclease resistance. A representative list of preferred modified sugars includes but is not limited to bicyclic modified sugars (BNAs), including LNA and ENA (4′-(CH2)2-0-2′ bridge); and substituted sugars, especially 2′-substituted sugars having a 2′-F, 2′—OCH2 or a 2′-0 (CH2)2—OCH3 substituent group. Sugars can also be replaced with sugar mimetic groups, among others. Methods for the preparations of modified sugars are well known to those skilled in the art. Suitable compounds can comprise one of the following at the 2′ position: OH; F; O—, S-, or N-alkyl; O—, S-, or N-alkenyl; 0-, S- or N-alkynyl; or O-alkyl-O-alkyl, wherein the alkyl, alkenyl and alkynyl can be substituted or unsubstituted CI to CIO alkyl or C2 to CIO alkenyl and alkynyl. Also suitable are O((CH2)nO) mCH3, O(CH2)nOCH3, O(CH2)nNH2, O(CH2)nCH3, O(CH2)nONH2, and O(CH2)nON((CH2)nCH3)2, where n and m are from 1 to about 10. Other oligonucleotides comprise one of the following at the 2′ position: CI to CIO lower alkyl, substituted lower alkyl, alkenyl, alkynyl, alkaryl, aralkyl, O-alkaryl or O-aralkyl, SH, SCH3, OCN, CI, Br, CN, CF3, OCF3, SOCH3, SO2CH3, ONO2, NO2, N3, NH2, heterocycloalkyl, heterocycloalkaryl, amino alkyl amino, poly-alkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. One modification includes 2′-methoxyethoxy (2′-O-CH2CH2OCH3, also known as 2′-O-(2-methoxyethyl) or 2′-MOE) (Martin et al., Helv. Chim. Acta, 1995, 78, 486-504), i.e., an alkoxyalkoxy group. A further modification includes 2′-dimethylaminooxyethoxy, i.e., a O(CH2)2ON(CH3)2 group, also known as 2′-DMAOE, and 2′-dimethylaminoethoxyethoxy (also known in the art as 2′-O-dimethyl-amino-ethoxy-ethyl or 2′-DMAEOE), i.e., 2′-O-(CH2)2-O-(CH2)2—N(CH3)2. Other modifications include 2′-methoxy (2′-O-CH3), 2′-aminopropoxy (2′—OCH2CH2CH2NH2), 2′-allyl (2′-CH2-CH-CH2), 2′-O-allyl (2′-O-CH2-CH-CH2) and 2′-fluoro (2′-F). The 2′-modification can be in the arabino (up) position or ribo (down) position. One 2′-arabino modification is 2′-F. Similar modifications can also be made at other positions on the oligonucleotide, particularly the 3′ position of the sugar on the 3′ terminal nucleotide or in 2′-5′ linked oligonucleotides and the 5′ position of 5′ terminal nucleotide. Antisense oligonucleotides can also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5, 118,800; 5,319,080; 5,359,044; 5,393,878; 5,446, 137; 5,466,786; 5,514,785; 5,519, 134; 5,567,811; 5,576,427; 5,591,722; 5,597,909; 5,610,300; 5,627,053; 5,639,873; 5,646,265; 5,658,873; 5,670,633; 5,792,747; 5,700,920; and, 6,147,200.

Other useful RNA derivatives incorporate nucleotides having modified carbohydrate moieties, such as 2′O-alkylated residues or 2′-O-methyl ribosyl derivatives and 2′-O-fluoro ribosyl derivatives or 2′-O,4′-constrained 2′-ethyl nucleoside or 2′-0, 4′-ethylene nucleoside. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated. Preferred siRNA modifications include 2′-deoxy-2′-fluorouridine or locked nucleic acid (LNA) nucleotides and RNA duplexes containing either phosphodiester or varying numbers of phosphorothioate linkages. Such modifications are known to one skilled in the art and are described, for example, in Braasch et al., Biochemistry, 42:7967-7975, 2003; Morita et al., Bioorg Med Chem Lett. 12 (1); 2002.

In one aspect of the technology described herein, oligomeric compounds include nucleosides modified to induce a 3′-endo sugar conformation. A nucleoside can incorporate modifications of the heterocyclic base, the sugar moiety or both to induce a desired 3′-endo sugar conformation. These modified nucleosides are used to mimic RNA-like nucleosides so that particular properties of an oligomeric compound can be enhanced while maintaining the desirable 3′-endo conformational geometry.

The monomers of the oligonucleotides described herein are coupled together via linkage groups. Suitably, each monomer is linked to the 3 ′adjacent monomer via a linkage group. The terms “linkage group” or “internucleoside linkage” mean a group capable of covalently coupling together two contiguous nucleoside monomers. Specific and preferred examples include phosphate groups (forming a phosphodiester between adjacent nucleoside monomers) and phosphorothioate groups (forming a phosphorothioate linkage between adjacent nucleoside monomers). Suitable linkage groups include those listed in WO 2007/031091, for example the linkage groups listed in the first paragraph of page 34 of WO 2007/031091 (hereby incorporated by reference). Phosphorothioate backbone modifications are well known in the art, see, for example, Hyjek-Skladanowska et. al., 2020 J. Am Chem. 142 (16), 7456-7468. It is, in various embodiments, preferred to modify the linkage group from its normal phosphodiester to one that is more resistant to nuclease attack, such as phosphorothioate or boranophosphate—these two, being cleavable by RNase H, permitting RNase-mediated antisense inhibition of expression of the target gene. In some embodiments, suitable sulphur(S) containing linkage groups as provided herein are preferred. In various embodiments, phosphorothioate linkage groups are preferred.

While ASOs that target splicing elements of the STAG1 transcript to interfere with normal splicing or that promote defective splicing are described herein, ASOs that target the STAG1 transcript for degradation, e.g., via RNAse H, are also contemplated. Thus, in certain embodiments, phosphorothioate linkages are used to link together monomers in the flanking regions of gapmers, which comprise antisense DNA sequence flanked by modified nucleotides (e.g., LNA) or ribonucleotide mimics, and which stimulate RNAseH-mediated degradation of target RNAs. In various embodiments, the flanking regions or gap region of a gapmer comprise linkage groups other than phosphorothioate, such as phosphodiester linkages, particularly, for instance when the use of nucleoside analogues protects the linkage groups within the flanking regions from endonuclease degradation-such as when the flanking regions comprise LNA monomers. It is recognized that the inclusion of phosphodiester linkages, such as one or two linkages, into an oligonucleotide with a phosphorothioate backbone, particularly with phosphorothioate linkage groups between or adjacent to nucleoside analogue monomers (typically in gapmer flanking regions), can modify the bioavailability and/or bio-distribution of an oligonucleotide-see WO 2008/053314, hereby incorporated by reference.

In some embodiments, such as the embodiments referred to above, where suitable and not specifically indicated, all remaining linkage groups can be either phosphodiester or phosphorothioate, or a mixture thereof. In some embodiments, all of the internucleoside linkage groups are phosphorothioate.

### Target Diseases or Disorders

STAG1-targeting ASOs as described herein can be used to treat any disease, disorder or cancer involving cells that are STAG2-deficient. STAG2 mutational inactivation is common in, e.g., MDS, AML, glioblastoma, urothelial carcinoma, and Ewing sarcoma. In some embodiments of any one of the aspects described herein, the disease or disorder is MDS or leukemia.

It is clear that reduction of STAG1 mRNA by less than 100% is effective to kill cohesin mutant or STAG2 mutant cells. In some embodiments, the contacting reduces STAG1 mRNA by at least 20%, at least 30%, at least 40%, at least 50%, at least 60%, at least 70%, at least 80% or at least 90%.

Myelodysplastic syndrome is a heterogeneous group of closely related clonal hematopoietic disorders that originate in an early blood-forming cell in the marrow. Such disorders are characterized by a cellular marrow with impaired morphology and maturation (dysmyelopoiesis) and peripheral blood cytopenias, resulting from ineffective blood cell production. In other words, the maturing blood cells often die in the marrow before they reach full maturity and enter the blood, accounting for the low blood cell concentrations. In patients suffering from myelodysplastic syndrome there may also be an accumulation of very immature marrow cells, referred to as leukemic blast cells. Patients with myelodysplastic syndrome experience symptoms including, but not limited to, fatigue, shortness of breath, unusual paleness (pallow), easy or unusual bruising ot bleeding, pinpoint-sized red spots beneath the skin, and frequent infections.

Diagnosis of myelodysplastic syndromes include blood tests to determine the number of red cells, white cells, and platelets and to look for unusual changes in the size, shape, and appearance of blood cells; as well as a bone marrow biopsy and aspiration to remove s small amount of liquid bone marrow and test for characteristics of the blood cells. Successful treatment of MDS could be indicated by the bone marrow and blood cell counts returning to normal or a normalization of blood cell characteristics as well as molecular remission and no further signs or symptoms of the disease.

MDS was previously known as preleukemia, and MDS may lead to acute myelogenous leukemia (AML). AML is a cancer of the blood and bone marrow. The disease rapidly progresses and mainly affects white blood cells called the myeloid cells, which normally develop into mature blood cells, including red blood cells, white blood cells, and platelets. AML results from uncontrolled blood cell production, where the bone marrow produces immature cells that develop into leukemic white blood cells called myeloblasts. These abnormal cells are unable to function properly and they build up and crowd out healthy cells.

Early signs and symptoms of AML include fever, bone pain, lethargy and fatigue, shortness of breath, pale skin, frequent infections, easy bruising, and unusual bleeding. AML can be diagnosed through blood tests to determine the number of white blood, red blood cells, and platelets. Patients with AML frequently have too many white blood cells, or not enough red blood cells or platelets. Furthermore, the presence of blast cells, immature cells found normally in bone marrow and not circulating in the blood, is another indicator of AML. Other methods of diagnoses include bone marrow tests, lumbar punctures, and subsequent laboratory testing for blood cell characteristics and for genetic mutations. Successful treatment of AML would be indicated by the bone marrow and blood cell counts returning to normal or a decrease in circulating blast cells, as well as molecular remission and no further signs or symptoms of the disease.

### Pharmaceutical Compositions, Administration and Efficacy

Methods known in the art for delivery of oligonucleotides or nucleic acids to cells in vivo can be adapted for use in methods and compositions described herein. While uptake of free oligonucleotides as described herein can be efficient, in some embodiments, an oligonucleotide as described herein can be covalently linked to a conjugated moiety to aid in delivery of the oligonucleotide across cell membranes. An example of a conjugated moiety that aids in delivery of the oligonucleotide across cell membranes is a lipophilic moiety, such as cholesterol. In various embodiments, an oligonucleotide as described herein is formulated with lipid formulations that form liposomes, such as Lipofectamine 2000™ or Lipofectamine RNAiMAX™, both of which are commercially available from Invitrogen. In some embodiments, the oligonucleotides described herein are formulated with a mixture of one or more lipid-like non-naturally occurring small molecules (“lipidoids”). Libraries of lipidoids can be synthesized by conventional synthetic chemistry methods and various amounts and combinations of lipidoids can be assayed in order to develop a vehicle for effective delivery of an oligonucleotide of a particular size to the targeted tissue by the chosen route of administration. Suitable lipidoid libraries and compositions can be found, for example in Akinc et al. (2008) Nature Biotech. 26:561-569 (2008), which is incorporated by reference herein. In the context of the present disclosure, “cellular uptake” refers to delivery and internalization of oligonucleotide compounds into cells. The oligonucleotide compounds can be internalized, for example, by cells grown in culture (in vitro), cells harvested from an animal (ex vivo) or by tissues following administration to an animal (in vivo).

Exemplary formulations which can be used for administering the oligonucleotide and/or dsRNA according to the present invention are discussed below.

The ASOs described herein can be formulated for delivery in a membranous molecular assembly, e.g., a liposome or a micelle. As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the ASO. The lipophilic material isolates the aqueous interior from an aqueous exterior, which typically does not include the ASO, although in some examples, it may. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include a ASO described herein are delivered into the cell. In some cases, the liposomes are also specifically targeted, e.g., to direct the conjugate to particular cell types.

A liposome containing a ASO described herein can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The ASO is then added to the micelles that include the lipid component. After condensation, the detergent is removed, e.g., by dialysis, to yield a liposomal preparation.

If necessary, a carrier compound that assists in condensation can be added during the condensation reaction, e.g., by controlled addition. For example, the carrier compound can be a polymer other than a nucleic acid (e.g., spermine or spermidine). pH can also be adjusted to favor condensation.

Further description of methods for producing stable polynucleotide or oligonucleotide delivery vehicles, which incorporate a polynucleotide/cationic lipid complex as structural components of the delivery vehicle, are described in, e.g., WO 96/37194. Liposome formation can also include one or more aspects of exemplary methods described in Felgner, P. L. et al., Proc. Natl. Acad. Sci., USA 8:7413-7417, 1987; U.S. Pat. Nos. 4,897,355; 5,171,678; Bangham, et al. M. Mol. Biol. 23:238, 1965; Olson, et al. Biochim. Biophys. Acta 557:9, 1979; Szoka, et al. Proc. Natl. Acad. Sci. 75:4194, 1978; Mayhew, et al. Biochim. Biophys. Acta 775:169, 1984; Kim, et al. Biochim. Biophys. Acta 728:339, 1983; and Fukunaga, et al. Endocrinol. 115:757, 1984, which are incorporated by reference in their entirety. Commonly used techniques for preparing lipid aggregates of appropriate size for use as delivery vehicles include sonication and freeze-thaw plus extrusion (see, e.g., Mayer, et al. Biochim. Biophys. Acta 858:161, 1986, which is incorporated by reference in its entirety). Microfluidization can be used when consistently small (50 to 200 nm) and relatively uniform aggregates are desired (Mayhew, et al. Biochim. Biophys. Acta 775:169, 1984, which is incorporated by reference in its entirety).

Liposomes that are pH-sensitive or negatively-charged entrap nucleic acid molecules rather than complex with them. Since both the nucleic acid molecules and the lipid are similarly charged, repulsion rather than complex formation occurs. Nevertheless, some nucleic acid molecules are entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 19, (1992)269-274, which is incorporated by reference in its entirety).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and include U.S. Pat. Nos. 5,283,185; 5,171,678; WO 94/00569; WO 93/24640; WO 91/16024; Felgner, J. Biol. Chem. 269:2550, 1994; Nabel, Proc. Natl. Acad. Sci. 90:11307, 1993; Nabel, Human Gene Ther. 3:649, 1992; Gershon, Biochem. 32:7143, 1993; and Strauss EMBO J. 11:417, 1992.

Cationic liposomes may also be used. Cationic liposomes possess the advantage of being able to fuse to the cell membrane. Non-cationic liposomes, although not able to fuse as efficiently with the plasma membrane, are taken up by macrophages in vivo and can be used to deliver siRNAs to macrophages.

Further advantages of liposomes include: liposomes obtained from natural phospholipids are biocompatible and biodegradable; liposomes can incorporate a wide range of water and lipid soluble drugs; liposomes can protect encapsulated siRNAs in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy) propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of siRNA (see, e.g., Felgner, P. L. et al., Proc. Natl. Acad. Sci., USA 8:7413-7417, 1987 and U.S. Pat. No. 4,897,355 for a description of DOTMA and its use with DNA, which are incorporated by reference in their entirety).

A DOTMA analogue, 1,2-bis (oleoyloxy)-3-(trimethylammonia) propane (DOTAP) can be used in combination with a phospholipid to form DNA-complexing vesicles. Lipofectin™ Bethesda Research Laboratories, Gaithersburg, Md.) is an effective agent for the delivery of highly anionic nucleic acids into living tissue culture cells that comprise positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis (oleoyloxy)-3,3-(trimethylammonia) propane (“DOTAP”) (Boehringer Mannheim, Indianapolis, Indiana) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (Transfectam™, Promega, Madison, Wisconsin) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., Biochim. Biophys. Res. Commun. 179:280, 1991). Lipopolylysine, made by conjugating polylysine to DOPE, has been reported to be effective for transfection in the presence of serum (Zhou, X. et al., Biochim. Biophys. Acta 1065:8, 1991, which is incorporated by reference in its entirety). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, California) and Lipofectamine (DOSPA) (Life Technology, Inc., Gaithersburg, Maryland). Other cationic lipids suitable for the delivery of oligonucleotides are described in WO 98/39359 and WO 96/37194.

Liposomes that include oligonucleotide and/or ASOs described herein can be made highly deformable. Such deformability can enable the liposomes to penetrate through pore that are smaller than the average radius of the liposome. For example, transfersomes are a type of deformable liposomes. Transfersomes can be made by adding surface edge activators, usually surfactants, to a standard liposomal composition. Transfersomes that include oligonucleotide and/or ASOs described herein can be delivered, for example, subcutaneously by infection in order to deliver ASOs to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application no PCT/US2007/080331, filed Oct. 3, 2007 also describes formulations that are amenable to the present invention.

Another example of a liposome is a lipid nanoparticle (LNP). As used herein, the term “LNP” refers to a stable nucleic acid-lipid particle. LNPs contain a cationic lipid, a non-cationic lipid, and a lipid that prevents aggregation of the particle (e.g., a PEG-lipid conjugate). LNPs are extremely useful for systemic applications, as they exhibit extended circulation lifetimes following intravenous (i.v.) injection and accumulate at distal sites (e.g., sites physically separated from the administration site).

ASOs as described herein can be used alone or in combination with other therapies, including chemotherapy, radiation, cancer immunotherapy, or combinations thereof. Such therapies can either directly target a tumor (e.g., by inhibition of a tumor cell protein or killing of highly mitotic cells) or act indirectly, e.g., to provoke or accentuate an anti-tumor immune response. Anti-cancer therapies which damage DNA to a lesser extent than chemotherapy may have efficacy. Examples of such therapies include radiation therapy, immunotherapy, hormone therapy, and gene therapy. Such therapies include, but are not limited to, the use of antisense polynucleotides, ribozymes, RNA interference molecules, triple helix polynucleotides and the like, where the nucleotide sequence of such compounds are related to the nucleotide sequences of DNA and/or RNA of genes that are linked to the initiation, progression, and/or pathology of a tumor or cancer. For example, oncogenes, growth factor genes, growth factor receptor genes, cell cycle genes, DNA repair genes, and others, may be used in such therapies.

The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (I-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

Immunotherapy may comprise, for example, use of cancer vaccines and/or sensitized antigen presenting cells. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines.

Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

DNA damage response inhibitors are widely used anti-cancer agents that have potent activity against tumor cells with deficiencies in various DNA damage response proteins. Inhibitors of proteins in this pathway target genes including, but not limited to PARP, DNA-PK, WEE1, CHK1/2, ATR, or ATM. Inhibitors of the DNA damage response are known in the field, see, for example Carlsen et al., 2022 Sec. Radiation Oncology 12, which is incorporated in its entirety, herein.

In preferred embodiments, ASOs are used in combination with one or more PARP inhibitors.

A variety of different PARP inhibitors are known, and any of them are contemplated for use in combination with STAG1-targeting antisense oligonucleotides as described herein for the treatment of MDS or cancer, e.g., cohesin-mutant or STAG2-mutant cancer. In one embodiment, the inhibitor of the DNA damage response is selected from talazoparib, veleparib, pamiparib, olaparib, rucaparib and niraparib. While combination of STAG1-targeting antisense oligonucleotides with a PARP inhibitor or other inhibitor of the DNA damage response can provide additive and/or potentially synergistic therapeutic benefit, it is also contemplated that the antisense oligonucleotides as described herein can be administered in combination with other anti-cancer therapeutics, including, but not limited to chemotherapeutic agents.

PARP has an essential role in facilitating DNA repair, controlling RNA transcription, mediating cell death, and regulating immune response. PARP inhibitors effectively target cells with reduced capacity for homologous recombination repair. Treatment with a double-strand break (DSB) repair inhibitor may render cells with intact homologous recombination machinery susceptible to PARP inhibitors, may enhance the efficacy of PARP inhibitors in homologous recombination deficient cancers, and may circumvent cases of acquired resistance to PARP inhibitors. An example of a PARP inhibitor includes, but is not limited to Iniparib (BSI 201; 4-iodo-3-nitrobenzamide), Olaparib (AZD-2281; KU-59436; 4-[(3-[(4-cyclopropylcarbonyl) piperazin-4-yl]carbonyl)-4-fluorophenyl]methyl (2H) phthalazin-1-one), Rucaparib (AG014699, PF-01367338; 2-{4-[(methylamino) methyl]phenyl}-1,3,4,5-tetrahydro-6H-azepino [5,4,3-cd]indol-6-one), Veliparib (ABT-888; 2-((R)-2-Methylpyrrolidin-2-yl)-1H-benzimidazole-4-carboxamide); CEP-8983; CEP-9722; MK-4827 (Niraparib; 2-{4-[(3S)-Piperidin-3-yl]phenyl}-2H-indazole-7-carboxamide); BMN-673 (LT-673; (8S,9R)-5-fluoro-8-(4-fluorophenyl)-9-(1-methyl-1H-1,2,4-triazol-5-yl)-8,9-dihydro-2H-pyrido[4,3,2-de]phthalazin-3 (7H)-one); LT-674; LT-628; 3-aminobenzamide (INO-1001; 3-AB); PD128763 (3,4-dihydro-5-methyl-1 (2H)-isoquinolinone); NU1025 (8-Hydroxy-2-methyl-4 (3H)-quinazolinone); DR 2313 (1,5,7,8-Tetrahydro-2-methyl-4H-thiopyrano[4,3-d]pyrimidin-4-one); UPF 1069 (5-(2-Oxo-2-phenylethoxy)-3,4-dihydroisoquinolin-1 (2H)-one); EB 47 (5′-Deoxy-5′-[4-[2-[(2,3-Dihydro-1-oxo-1H-isoindol-4-yl)amino]-2-oxoethyl]-1-piperazinyl]-5′-oxoadenosine dihydrochloride); E7016 (Benzopyrano[4,3,2-de]phthalazin-3 (2H)-one, 10-[(4-hydroxy-1-piperidinyl)methyl]-); 4-HQN (4-(1H)-Quinazolinone); ABT-767 and compounds as described in Griffin et al 1998, J. Med. Chem. 41, 5247; Skalitzky et al. 2003, J. Med. Chem. 46:210-213; Zaremba et al. 2007, Anti-Cancer Agents in Medicinal Chemistry 7, 515; Lewis et al. 2007, Curr Opin. Investigational Drugs 8, 1061; Guha 2011, Nature Biotechnology 29, 373-374; Rouleau et al. 2010, Nature Reviews Cancer 10, 293-301; Miknyoczki et al., 2007 Mol Cancer Ther, 6 (8), 2290-2302; Pellicciari et al. 2008, Chem. Med. Chem 3, 91; Jones et al., 2009, J Med Chem, 52 (22), 7170-7185; Mason et al. 2008, Invest New Drugs, 26 (1), 1-5; Ferraris et al., 2010, J. Med. Chem. 53 4561; US patent applications: US2006/0229289; US20070259937; US20120309717; US20130011365; US patents: U.S. Pat. Nos. 8,372,987; 8,362,030; 8,236,802; 8,217,070; 8,183,250; 8,088,760; international patent applications: WO 01/85686; WO 00/42040; WO 00/39070; WO 00/39104; WO 99/11623; WO 99/11628; WO 99/11622; WO 99/59975; WO 99/11644; WO 99/11945; WO 99/11649; and WO 99/59973, each of which is herein incorporated by reference in its entirety.

In addition, other anti-cancer agents that can be used in combination ASOs as described herein include alkylating agents such as thiotepa and CYTOXAN™; cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas, such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN™, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK; polysaccharide complex (JHS Natural Products™, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL™ paclitaxel (Bristol-Meyers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE™ doxetaxel (Rhone-Poulenc Rorer, Antony, France); chloranbucil; GEMZAR™, gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE™, vinorelbine; novantrone; teniposide; edatrexate; daunomycin; aminopterin; xeloda; ibandronate; irinotecan (CAMPTOSAR™, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX™); lapatinib (TYKERB™); inhibitors of PKC-alpha, Raf, H-Ras, EGFR (e.g., erlotinib (TARCEVA™)) and VEGF-A that reduce cell proliferation, and pharmaceutically acceptable salts, acids or derivatives of any of the above. In addition, the methods of treatment can further include the use of radiation.

Pharmaceutical or therapeutic compositions comprising a therapeutic agent for the treatment of cancer can contain a physiologically tolerable carrier, wherein the therapeutic agent is dissolved or dispersed therein as an active ingredient(s). In a preferred embodiment, the pharmaceutical composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological or pharmaceutical composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition comprising a therapeutic agent for treatment of cancer or other disease or disorder involving STAG2-deficient cells can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of cancer or other disease or disorder involving STAG2-deficient cells will depend on the nature of the disorder or condition, and can be determined by standard clinical techniques.

A pharmaceutical composition as described herein can be formulated for parenteral administration, e.g., by bolus injection or continuous infusion. Formulations for injection can be presented in unit dosage form, e.g., in ampoules or in multidose containers with, optionally, an added preservative. The compositions can be suspensions, solutions, or emulsions in oily or aqueous vehicles, and can contain formulatory agents such as suspending, stabilizing, and/or dispersing agents.

Pharmaceutical compositions for parenteral administration include aqueous solutions of the active preparation in water-soluble form. Additionally, suspensions of the active ingredients can be prepared as appropriate oily or water-based injection suspensions.

Suitable lipophilic solvents or vehicles include fatty oils such as sesame oil, or synthetic fatty acid esters such as ethyl oleate, triglycerides, or liposomes. Aqueous injection suspensions can contain substances that increase the viscosity of the suspension, such as sodium carboxymethyl cellulose, sorbitol, or dextran.

Optionally, the suspension can also contain suitable stabilizers or agents that increase the solubility of the active ingredients, to allow for the preparation of highly concentrated solutions. Alternatively, the active ingredient can be in powder form for constitution with a suitable vehicle, e.g., a sterile, pyrogen-free, water-based solution, before use.

In some embodiments, a therapeutic agent can be delivered in an immediate release form. In other embodiments, the therapeutic agent can be delivered in a controlled-release system or sustained-release system. Controlled- or sustained-release pharmaceutical compositions can have a common goal of improving drug therapy over the results achieved by their non-controlled or non-sustained-release counterparts. Advantages of controlled- or sustained-release compositions include extended activity of the therapeutic agents, reduced dosage frequency, and increased compliance. In addition, controlled- or sustained-release compositions can favorably affect the time of onset of action or other characteristics, such as blood levels of the therapeutic agent, and can thus reduce the occurrence of adverse side effects. Controlled- or sustained-release of an active ingredient can be stimulated by various conditions, including but not limited to, changes in pH, changes in temperature, concentration or availability of enzymes, concentration or availability of water, or other physiological conditions or compounds.

The appropriate dosage range for a given therapeutic agent depends upon the potency, and includes amounts large enough to produce the desired effect, e.g., reduction in at least one symptom of cancer. The dosage of the therapeutic agent should not be so large as to cause unacceptable or life-threatening adverse side effects or should be used under close supervision by a medical professional. Generally, the dosage will vary with the type of anti-cancer agent, and with the age, condition, and sex of the patient. The dosage can be determined by one of skill in the art and can also be adjusted by the individual physician in the event of any complication.

Typically, the dosage of a given therapeutic can range from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 μg/kg body weight to 30 μg/kg body weight. Alternatively, the dose range will be titrated to maintain serum levels between 5 μg/mL and 30 μg/mL.

Currently available therapies, including experimental therapies, for cancer or a symptom thereof and their dosages, routes of administration and recommended usage are known in the art and/or have been described in such literature as the Physician's Desk Reference (60th ed., 2017). With respect to experimental therapies, an appropriate dosage can be estimated based on dose-response modeling in animal models or in silico modeling of drug effects.

Administration of the doses recited above or as employed by a skilled clinician can be repeated for a limited and defined period of time. In some embodiments, the doses are given once a day, or multiple times a day, for example, but not limited to three times a day. Typically, the dosage regimen is informed by the half-life of the agent as well as the minimum therapeutic concentration of the agent in blood, serum or localized in a given biological tissue. In a preferred embodiment, the doses recited above are administered daily for several weeks or months. The duration of treatment depends upon the subject's clinical progress and continued responsiveness to therapy. Continuous, relatively low maintenance doses are contemplated after an initial higher therapeutic dose.

A therapeutically effective amount is an amount of an agent that is sufficient to produce a statistically significant, measurable change of a given symptom of cancer. Such effective amounts can be gauged in clinical trials as well as animal studies for a given agent. For example, reduction of a given symptom of cancer can be indicative of adequate therapeutic efficacy of an agent(s).

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. The agent can be administered systemically, if so desired.

Therapeutic compositions containing at least one therapeutic agent can be conventionally administered in a unit dose. The term “unit dose” when used in reference to a therapeutic composition refers to physically discrete units suitable as unitary dosage for the subject, each unit containing a predetermined quantity of a therapeutic agent calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

In some embodiments, a combination of anti-cancer therapeutic agents is used in the treatment of cancer in a subject diagnosed as described herein.

In some embodiments, a therapeutically effective agent is administered to a subject concurrently with a combination therapy. As used herein, the term “concurrently” is not limited to the administration of the two or more agents at exactly the same time, but rather, it is meant that they are administered to a subject in a sequence and within a time interval such that they can act together (e.g., synergistically to provide an increased benefit than if they were administered otherwise). For example, the combination of therapeutics can be administered at the same time or sequentially in any order at different points in time; however, if not administered at the same time, they should be administered sufficiently close in time so as to provide the desired therapeutic effect, preferably in a synergistic fashion. The agents can be administered separately, in any appropriate form and by any suitable route. When each of the therapeutic agents in a combination are not administered in the same pharmaceutical composition, it is understood that they can be administered in any order to a subject in need thereof. For example, the first therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second therapeutic agent, to a subject in need thereof (or vice versa). In other embodiments, the delivery of either therapeutic agent ends before the delivery of the other agent/treatment begins. In some embodiments of either case, the treatment is more effective because of combined administration. For example, the therapeutic agents used in combination are more effective than would be seen with either agent alone. In some embodiments, delivery is such that the reduction in a symptom, or other parameter related to the disorder is greater than what would be observed with either therapeutic agent alone. The effect of such a combination can be partially additive, wholly additive, or greater than additive. The agent and/or other therapeutic agents, procedures or modalities can be administered during periods of active disease, or during a period of persistence or less active disease.

When administered in combination, one or more of the therapeutic agents can be administered in an amount or dose that is higher, lower or the same as the amount or dosage of the given agent used individually, e.g., as a monotherapy. In certain embodiments, the administered amount or dosage of a first therapeutic agent when administered in combination with a second therapeutic agent is lower (e.g., at least 20%, at least 30%, at least 40%, or at least 50%) than the amount or dosage of the first agent when used individually. In other embodiments, the amount or dosage of a first therapeutic agent, when administered in combination with a second therapeutic agent, results in a desired effect (e.g., improved cognitive functioning) is lower (e.g., at least 20%, at least 30%, at least 40%, or at least 50% lower) than the amount or dosage of the first (or second) agent required to achieve the same therapeutic effect when administered alone.

The efficacy of a given treatment for cancer can be determined by the skilled clinician. However, a treatment is considered “effective treatment,” as the term is used herein, if any one or all of the signs or symptoms of cancer is/are altered in a beneficial manner, or other clinically accepted symptoms or markers of disease are improved, or ameliorated, e.g., by at least 10% following treatment with a therapeutic agent for cancer. Efficacy can also be measured by failure of an individual to worsen as assessed by stabilization of the disease, or the need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing progression of the cancer; or (2) relieving the disease, e.g., causing regression of symptoms; and (3) preventing or reducing the likelihood of the development of the disease, or preventing secondary diseases/disorders associated with the infection.

An effective amount for the treatment of a disease means that amount which, when administered to a mammal in need thereof, is sufficient to result in effective treatment as that term is defined herein, for that disease. Efficacy of an agent can be determined by assessing physical indicators of the disease, such as e.g., anemia, white blood cell levels or identity, pain, fatigue, fever, etc.

The treatment according to the methods provided herein can reduce or eliminate one or more symptoms associated with cancer such as fatigue, pain, tumor size, tumor growth, etc. In one embodiment, the cancer is prostate cancer and the one or more symptoms associated with prostate cancer include trouble urinating, increased frequency of urination, pelvic pain or discomfort, decreased force of urination, difficulty starting or stopping urine stream, blood in semen, and bone pain.

The technology provided herein can further be defined by the following numbered paragraphs.

TABLE 1
Examples of ASOs targeting STAG1 expression

| Annotation | SEQ ID Nos | Sequence |
|---|---|---|
| E3_5 unmodified | SEQ ID NO: | TCTTCAAGCTCGCTGCCAGCA |
| | 1 | |
| E3_6 unmodified | SEQ ID NO: | TTCTTCAAGCTCGCTGCCAGC |
| | 2 | |
| E3_7 unmodified | SEQ ID NO: | CAAGCTCGCTGCCAGCATCGG |
| | 3 | |
| E3_8 unmodified | SEQ ID NO: | TCAAGCTCGCTGCCAGCATCG |
| | 4 | |
| E3_9 unmodified | SEQ ID NO: | CTTCAAGCTCGCTGCCAGCAT |
| | 5 | |
| E3_10 unmodified | SEQ ID NO: | TTCAAGCTCGCTGCCAGCATC |
| | 6 | |
| E3_11 unmodified | SEQ ID NO: | TTTCTTCAAGCTCGCTGCCAG |
| | 7 | |
| E5_6 unmodified | SEQ ID NO: | GATTCAATCCAGTCATCCACC |
| | 8 | |
| E5_7 unmodified | SEQ ID NO: | TGATTCAATCCAGTCATCCAC |
| | 9 | |
| E5_8 unmodified | SEQ ID NO: | TCAATCCAGTCATCCACCACG |
| | 10 | |
| E5_9 unmodified | SEQ ID NO: | AATCCAGTCATCCACCACGGA |
| | 11 | |
| E5_10 unmodified | SEQ ID NO: | TTCAATCCAGTCATCCACCAC |
| | 12 | |
| E5_11 unmodified | SEQ ID NO: | TGCGATGTCCCTGTCTTGTTT |
| | 13 | |
| E5_12 unmodified | SEQ ID NO: | GCGATGTCCCTGTCTTGTTTA |
| | 14 | |
| E29_2 unmodified | SEQ ID NO: | TCCTTTCCATCATCTGCTCGG |
| | 15 | |
| E29_3 unmodified | SEQ ID NO: | CCTTTCCATCATCTGCTCGGT |
| | 16 | |
| E29_4 unmodified | SEQ ID NO: | CTCCTTTCCATCATCTGCTCG |
| | 17 | |
| E29_5 unmodified | SEQ ID NO: | CCTCCTTTCCATCATCTGCTC |
| | 18 | |
| E29_6 unmodified | SEQ ID NO: | TCCATCATCTGCTCGGTAAGG |
| | 19 | |
| E29_7 unmodified | SEQ ID NO: | CCCTCCTTTCCATCATCTGCT |
| | 20 | |
| E3_5 with PS bonds | SEQ ID NO: | T*C*T*T*C*A*A*G*C*T*C*G*C*T*G |
| | 21 | *C*C*A*G*C*A |
| E3_6 with PS bonds | SEQ ID NO: | T*T*C*T*T*C*A*A*G*C*T*C*G*C*T* |
| | 22 | G*C*C*A*G*C |
| E3_7 with PS bonds | SEQ ID NO: | C*A*A*G*C*T*C*G*C*T*G*C*C*A*G |
| | 23 | *C*A*T*C*G*G |
| E3_8 with PS bonds | SEQ ID NO: | T*C*A*A*G*C*T*C*G*C*T*G*C*C*A |
| | 24 | *G*C*A*T*C*G |
| E3_9 with PS bonds | SEQ ID NO: | C*T*T*C*A*A*G*C*T*C*G*C*T*G*C |
| | 25 | *C*A*G*C*A*T |
| E3_10 with PS bonds | SEQ ID NO: | T*T*C*A*A*G*C*T*C*G*C*T*G*C*C |
| | 26 | *A*G*C*A*T*C |
| E3_11 with PS bonds | SEQ ID NO: | T*T*T*C*T*T*C*A*A*G*C*T*C*G*C* |
| | 27 | T*G*C*C*A*G |
| E5_6 with PS bonds | SEQ ID NO: | G*A*T*T*C*A*A*T*C*C*A*G*T*C*A |
| | 28 | *T*C*C*A*C*C |
| E5_7 with PS bonds | SEQ ID NO: | T*G*A*T*T*C*A*A*T*C*C*A*G*T*C |
| | 29 | *A*T*C*C*A*C |
| E5_8 with PS bonds | SEQ ID NO: | T*C*A*A*T*C*C*A*G*T*C*A*T*C*C |
| | 30 | *A*C*C*A*C*G |
| E5_9 with PS bonds | SEQ ID NO: | A*A*T*C*C*A*G*T*C*A*T*C*C*A*C |
| | 31 | *C*A*C*G*G*A |
| E5_10 with PS bonds | SEQ ID NO: | T*T*C*A*A*T*C*C*A*G*T*C*A*T*C |
| | 32 | *C*A*C*C*A*C |
| E5_11 with PS bonds | SEQ ID NO: | T*G*C*G*A*T*G*T*C*C*C*T*G*T*C |
| | 33 | *T*T*G*T*T*T |
| E5_12 with PS bonds | SEQ ID NO: | G*C*G*A*T*G*T*C*C*C*T*G*T*C*T |
| | 34 | *T*G*T*T*T*A |
| E29_2 with PS bonds | SEQ ID NO: | T*C*C*T*T*T*C*C*A*T*C*A*T*C*T* |
| | 35 | G*C*T*C*G*G |
| E29_3 with PS bonds | SEQ ID NO: | C*C*T*T*T*C*C*A*T*C*A*T*C*T*G* |
| | 36 | C*T*C*G*G*T |
| E29_4 with PS bonds | SEQ ID NO: | C*T*C*C*T*T*T*C*C*A*T*C*A*T*C* |
| | 37 | T*G*C*T*C*G |
| E29_5 with PS bonds | SEQ ID NO: | C*C*T*C*C*T*T*T*C*C*A*T*C*A*T* |
| | 38 | C*T*G*C*T*C |
| E29_6 with PS bonds | SEQ ID NO: | T*C*C*A*T*C*A*T*C*T*G*C*T*C*G* |
| | 39 | G*T*A*A*G*G |
| E29_7 with PS bonds | SEQ ID NO: | C*C*C*T*C*C*T*T*T*C*C*A*T*C*A* |
| | 40 | T*C*T*G*C*T |
| E3_5 with 2′MOE modification | SEQ ID NO: | T*C*T*T*C*A*A*G*C*T*C*G*C*T*G |
| | 41 | *C*C*A*G*C*A |
| E3_6 with 2′MOE modification | SEQ ID NO: | T*T*C*T*T*C*A*A*G*C*T*C*G*C*T* |
| | 42 | G*C*C*A*G*C |
| E3_7 with 2′MOE modification | SEQ ID NO: | C*A*A*G*C*T*C*G*C*T*G*C*C*A*G |
| | 43 | *C*A*T*C*G*G |
| E3_8 with 2′MOE modification | SEQ ID NO: | T*C*A*A*G*C*T*C*G*C*T*G*C*C*A |
| | 44 | *G*C*A*T*C*G |
| E3_9 with 2′MOE modification | SEQ ID NO: | C*T*T*C*A*A*G*C*T*C*G*C*T*G*C |
| | 45 | *C*A*G*C*A*T |
| E3_10 with 2′MOE | SEQ ID NO: | T*T*C*A*A*G*C*T*C*G*C*T*G*C*C |
| modification | 46 | *A*G*C*A*T*C |
| E3_11 with 2′MOE | SEQ ID NO: | T*T*T*C*T*T*C*A*A*G*C*T*C*G*C* |
| modification | 47 | T*G*C*C*A*G |
| E5_6 with 2′MOE modification | SEQ ID NO: | G*A*T*T*C*A*A*T*C*C*A*G*T*C*A |
| | 48 | *T*C*C*A*C*C |
| E5_7 with 2′MOE modification | SEQ ID NO: | T*G*A*T*T*C*A*A*T*C*C*A*G*T*C |
| | 49 | *A*T*C*C*A*C |
| E5_8 with 2′MOE modification | SEQ ID NO: | T*C*A*A*T*C*C*A*G*T*C*A*T*C*C |
| | 50 | *A*C*C*A*C*G |
| E5_9 with 2′MOE modification | SEQ ID NO: | A*A*T*C*C*A*G*T*C*A*T*C*C*A*C |
| | 51 | *C*A*C*G*G*A |
| E5_10 with 2′MOE | SEQ ID NO: | T*T*C*A*A*T*C*C*A*G*T*C*A*T*C |
| modification | 52 | *C*A*C*C*A*C |
| E5_11 with 2′MOE | SEQ ID NO: | T*G*C*G*A*T*G*T*C*C*C*T*G*T*C |
| modification | 53 | *T*T*G*T*T*T |
| E5_12 with 2′MOE | SEQ ID NO: | G*C*G*A*T*G*T*C*C*C*T*G*T*C*T |
| modification | 54 | *T*G*T*T*T*A |
| E29_2 with 2′MOE | SEQ ID NO: | T*C*C*T*T*T*C*C*A*T*C*A*T*C*T* |
| modification | 55 | G*C*T*C*G*G |
| E29_3 with 2′MOE | SEQ ID NO: | C*C*T*T*T*C*C*A*T*C*A*T*C*T*G* |
| modification | 56 | C*T*C*G*G*T |
| E29_4 with 2′MOE | SEQ ID NO: | C*T*C*C*T*T*T*C*C*A*T*C*A*T*C* |
| modification | 57 | T*G*C*T*C*G |
| E29_5 with 2′MOE | SEQ ID NO: | C*C*T*C*C*T*T*T*C*C*A*T*C*A*T* |
| modification | 58 | C*T*G*C*T*C |
| E29_6 with 2′MOE | SEQ ID NO: | T*C*C*A*T*C*A*T*C*T*G*C*T*C*G* |
| modification | 59 | G*T*A*A*G*G |
| E29_7 with 2′MOE | SEQ ID NO: | C*C*C*T*C*C*T*T*T*C*C*A*T*C*A* |
| modification | 60 | T*C*T*G*C*T |
| E5_combo 11, 12 unmodified | SEQ ID NO: | TGCGATGTCCCTGTCTTGTTTA |
| | 61 | |
| E5_combo 11, 12 with PS | SEQ ID NO: | T*G*C*G*A*T*G*T*C*C*C*T*G*T*C |
| bonds | 62 | *T*T*G*T*T*T*A |
| E5_combo 11, 12 with 2′MOE | SEQ ID NO: | T*G*C*G*A*T*G*T*C*C*C*T*G*T*C |
| modification | 63 | *T*T*G*T*T*T*A |
| E5_13 unmodified | SEQ ID NO: | TGCGATGTCCCTGTCTTGT |
| | 64 | |
| E5_14 unmodified | SEQ ID NO: | TGCGATGTCCCTGTCTT |
| | 65 | |
| E5_15 unmodified | SEQ ID NO: | TGCGATGTCCCTGTC |
| | 66 | |
| E5_16 unmodified | SEQ ID NO: | GATGTCCCTGTCTTGTTTA |
| | 67 | |
| E5_17 unmodified | SEQ ID NO: | TGTCCCTGTCTTGTTTA |
| | 68 | |
| E5_18 unmodified | SEQ ID NO: | TCCCTGTCTTGTTTA |
| | 69 | |
| E5_13 with PS bonds | SEQ ID NO: | T*G*C*G*A*T*G*T*C*C*C*T*G*T*C |
| | 70 | *T*T*G*T |
| E5_14 with PS bonds | SEQ ID NO: | T*G*C*G*A*T*G*T*C*C*C*T*G*T*C |
| | 71 | *T*T |
| E5_15 with PS bonds | SEQ ID NO: | T*G*C*G*A*T*G*T*C*C*C*T*G*T*C |
| | 72 | |
| E5_16 with PS bonds | SEQ ID NO: | G*A*T*G*T*C*C*C*T*G*T*C*T*T*G* |
| | 73 | T*T*T*A |
| E5_17 with PS bonds | SEQ ID NO: | T*G*T*C*C*C*T*G*T*C*T*T*G*T*T* |
| | 74 | T*A |
| E5_18 with PS bonds | SEQ ID NO: | T*C*C*C*T*G*T*C*T*T*G*T*T*T*A |
| | 75 | |
| E5_13 with 2′ MOE | SEQ ID NO: | T*G*C*G*A*T*G*T*C*C*C*T*G*T*C |
| modification | 76 | *T*T*G*T |
| E5_14 with 2′ MOE | SEQ ID NO: | T*G*C*G*A*T*G*T*C*C*C*T*G*T*C |
| modification | 77 | *T*T |
| E5_15 with 2′ MOE | SEQ ID NO: | T*G*C*G*A*T*G*T*C*C*C*T*G*T*C |
| modification | 78 | |
| E5_16 with 2′ MOE | SEQ ID NO: | G*A*T*G*T*C*C*C*T*G*T*C*T*T*G* |
| modification | 79 | T*T*T*A |
| E5_17 with 2′ MOE | SEQ ID NO: | T*G*T*C*C*C*T*G*T*C*T*T*G*T*T* |
| modification | 80 | T*A |
| E5_18 with 2′ MOE | SEQ ID NO: | T*C*C*C*T*G*T*C*T*T*G*T*T*T*A |
| modification | 81 | |

Legend: 2′MOE modification indicated by underline. PS bond indicated by *

### EXAMPLES

### Example 1

STAG2 is the most frequently mutated cohesin subunit in AML (FIG. 2) and is also recurrently mutated in solid tumors. Indeed, STAG2 is one of only a dozen human genes to be significantly mutated in four or more distinct types of human cancer², with recurrent mutations in cancers including glioblastoma, breast cancer, bladder cancer, melanoma and Ewing sarcoma²-4. STAG2 is present on the X chromosome, thus mutations in males (or on the active X allele in females) often lead to a complete loss of STAG2 protein, and replacement in the cohesin complex by its paralog STAG1. Upon loss of STAG2, STAG1 becomes an essential protein. Importantly, this establishes an absolute dependency of STAG2-mutant cells on expression of STAG1, and both RNAi and CRISPR/Cas9 screens have highlighted STAG1 as a selective, critical dependency in STAG2 mutant cells (MS #1,¹³). Data from reports using next-generation sequencing in AML samples shows cohesin subunits SMC1A, SMC3, RAD21, STAG1, and STAG2 with frequent mutation. The core components of the cohesin complex are collectively mutated in 14% of patients with de novo AML. Missense mutations include inframe indels while truncating mutations include nonsense, frameshift, and splice site mutations. AML genomes have fewer mutations than most adult cancers, and among them, 13% correspond to cohesin-related genes. STAG2 is the most frequently mutated cohesin subunit in AML.

### Example 2

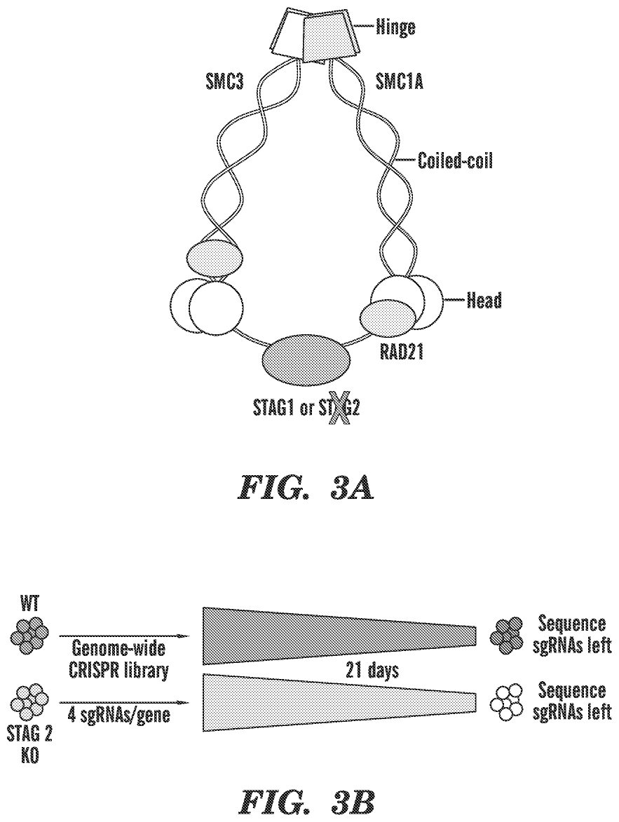

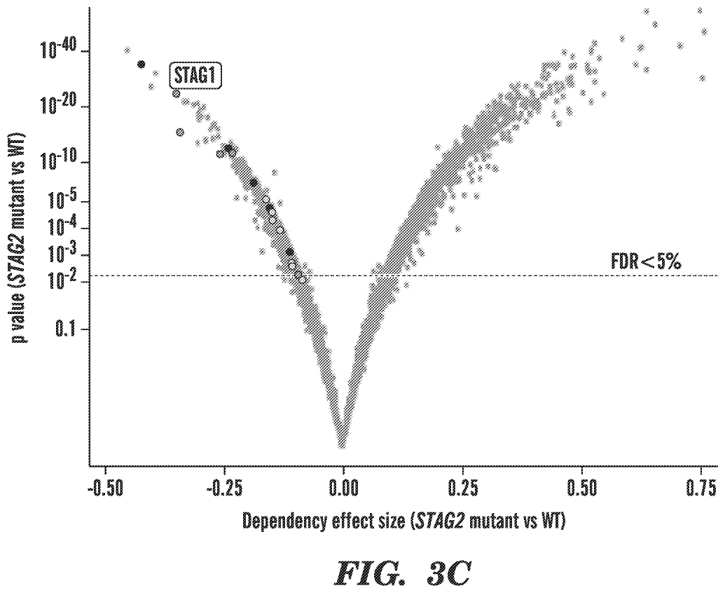

The inventors used an AML cell model with genetically engineered STAG2 KO that can recapitulate selective dependence on STAG1. STAG2 KO were generated using CRISPR/Cas9 (FIG. 3A) The inventors used a genome-scale CRISPR screen in WT or STAG2 KO U937 cell line using the Avana sgRNA library, which targets a total of 20,000 protein-coding genes with 4 unique sgRNAs per gene and includes 1000 nontargeting sgRNA controls (FIG. 3B). A volcano plot (FIG. 3C) depicting differential dependencies in STAG2-mutant versus WT cells shows composite data for 5 STAG2-mutant cell lines (U937 STAG2-KO2, STAG2-KO3, KOC5, KOD5C, KOG8B) and 6 STAG2-WT cell lines (U937 WT-1, WT-2, NCB1, NCB12, NCB2A, NCC4). Respective sets of genes representing dependency in STAG2-mutant over WT cells with FDR <5% are shown in highlighted dots. STAG2-mutant cells were strongly dependent on STAG1 (FIG. 3C). STAG1 knockout does not elicit phenotypes in WT U937 cells, or animal models.

### Example 3

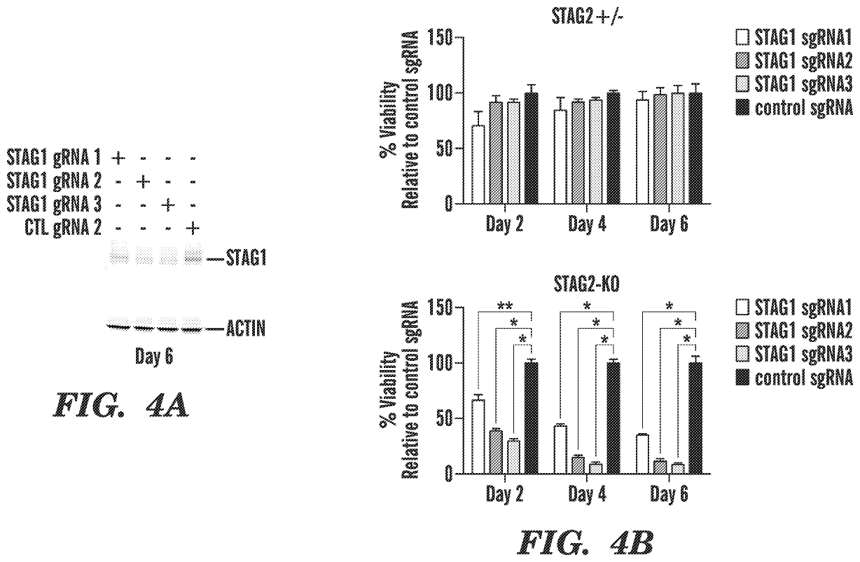

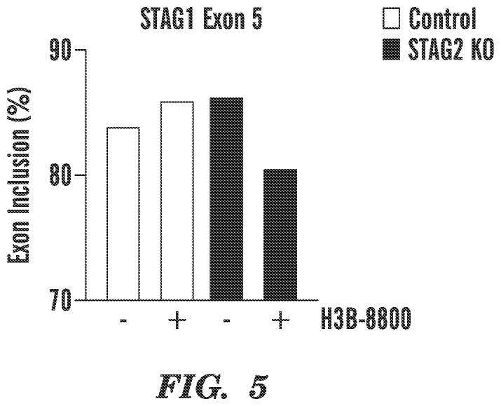

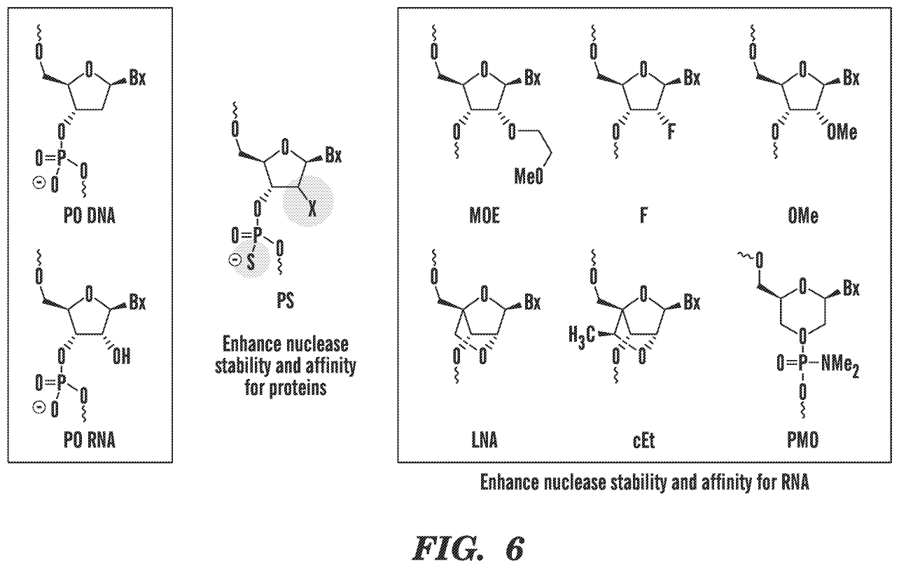

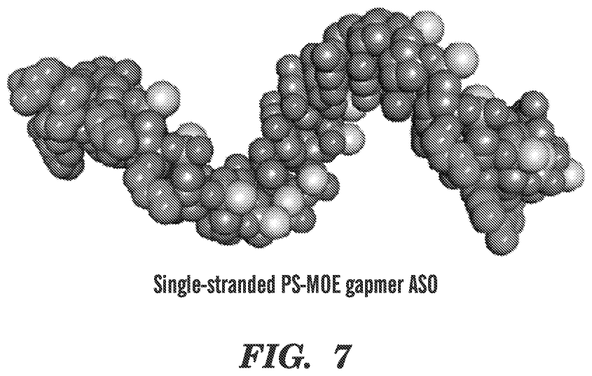

The inventors showed that STAG2 KO renders cell highly sensitive to reduction of STAG1 levels. Guide RNAs targeting STAG1 for CRISPR KO had variable effectiveness in control cells, reducing STAG1 protein level by 47%, 71%, and 81% respectively as measured by western blot using a STAG1 antibody. Actin served as a loading control. Effectiveness was determined as % of non-homologous end joining across the gRNA target site (FIG. 4A). Measurements of cell viability 2-6 days following knockdown of STAG1 using STAG1 sgRNA show that no STAG 1 gRNAs affected viability in STAG2 proficient cells; however, in STAG2 KO cells, knockdown of STAG1 greatly reduced cell viability, indicating that in the absence of STAG2, reduction of STAG1 by ˜50-75% greatly impacts those cells' viability (FIG. 4B).

### Example 4

The inventors show that cohesin mutations render AML cells highly sensitive to splicing inhibitors such as H3B-8800. Measurements of STAG1 Exon 5 inclusion percentage in control or STAG2 KO cells following treatment with H3B-8800 show that skipping of STAG1 Exon 5 would shift the coding sequence out of frame and cause RNA degradation by nonsense mediated RNA decay. Data show that STAG2 KO leads to increased STAG1 Exon 5 skipping following treatment of H3B-8800. These data indicate that splicing inhibitors have increased effectiveness in STAG2 KO cells.

### Example 5

Knowing that reduction of STAG1 mRNA selectively kills STAG2 mutant cells, the inventors sought to develop ASOs that cause mis-splicing and/or reduction of STAG1 mRNA. ASOs were designed with phosphorothioate (PS) backbone linkages to enable entering cells by ‘free uptake’. Father MOE modifications increase ASO stability to nucleases and binding to RNA targets. The inventors developed gapmers with a central region, or ‘gap’ that has DNS characteristics, flanked by modified RNA bases. The short central stretch of DNA forms an RNA-DNA hybrid with RNA target. This recruits RNaseH to degrade target RNA.

### Example 6

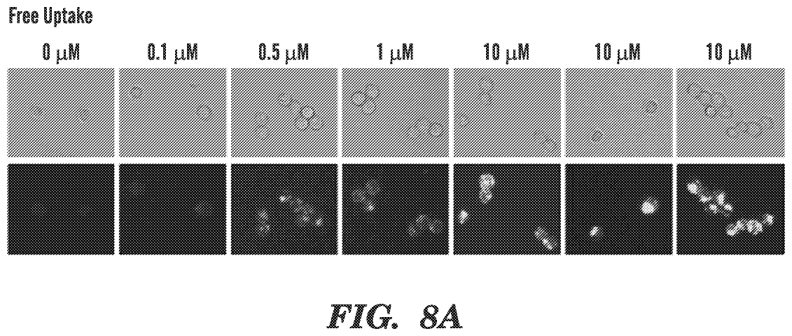

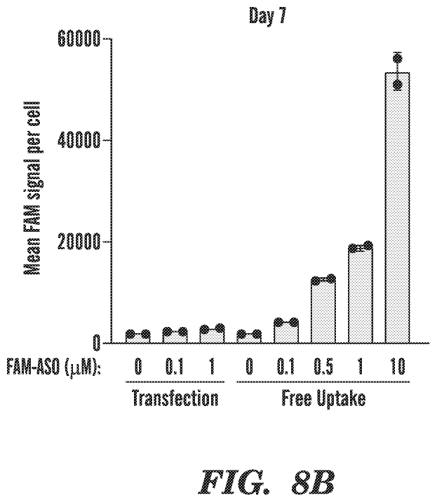

The inventors show uptake of fluorescently labeled ASO in HEK 293T cells. Fluorescently labeled gapmer ASOs including PS linkages and MOE base modifications were added to the media of HEK 293T cells at concentrations between 0 μM and 10 μM. Fluorescence was seen in cells indicating free uptake of the ASOs was successful (FIG. 8A). Measurement of the mean fluorescent signal per cell 7 days after transfection or free uptake of ASOs indicate successful uptake of ASOs that were added into the media of HEK 293T cells at concentrations ranging from 0.1 μM to 10 μM (FIG. 8B).

### Example 7

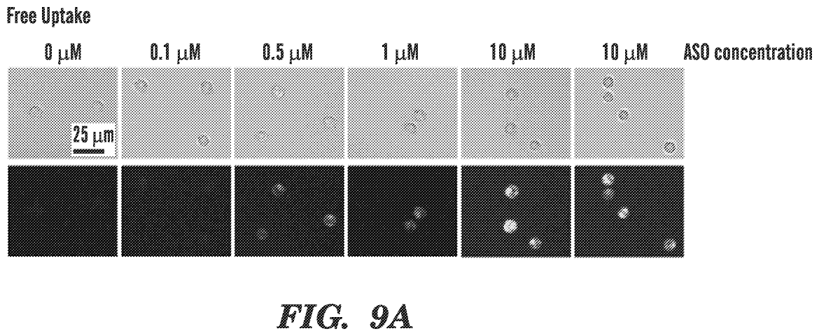

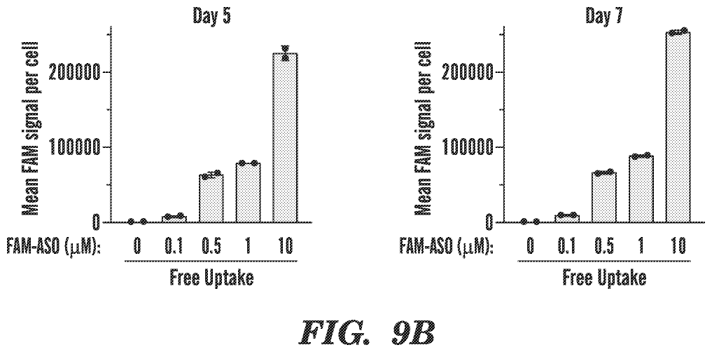

The inventors show uptake of fluorescently labeled ASOs in U937 AML cell line. Fluorescently labeled ASOs were added to the media of U937 AML cells at concentrations between 0 μM and 10 μM. Fluorescence was seen in cells indicating free uptake of the ASOs was successful (FIG. 9A). Measurement of the mean fluorescent signal per cell 5-7 days after fluorescently labeled ASOs were added to the media of U 937 AML cells indicate successful free uptake of ASOs at concentrations ranging from 0.1 μM to 10 μM (FIG. 9B).

### Example 8

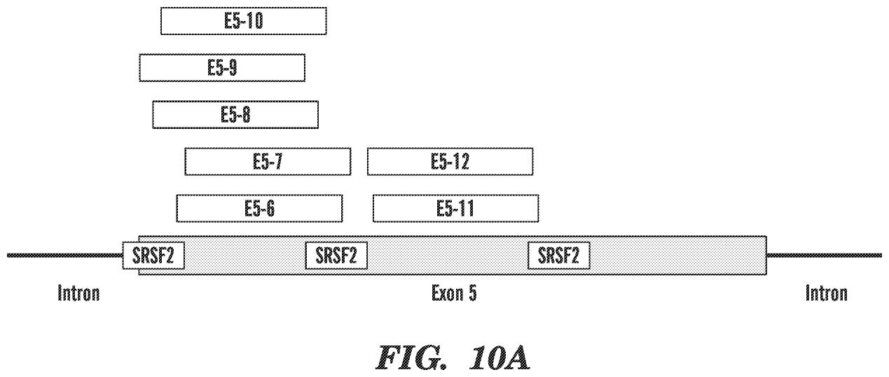

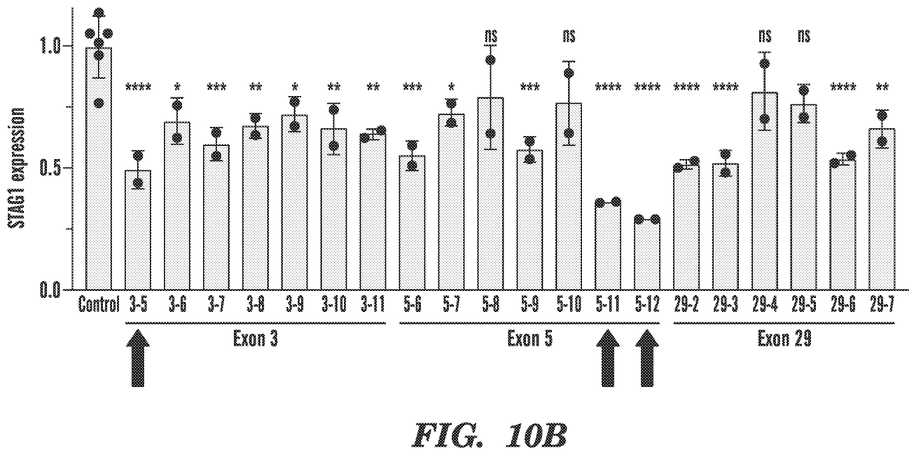

The inventors show ASOs targeting regions of STAG1 reduce STAG1 expression. They tiled of ASOs over STAG1 exons, specifically exons that showed mis-splicing in H3B-8800 treated cells. ASOs targeting these regions were designed to promote exon skipping and subsequent degradation of STAG1 transcripts. ASOs were designed against Exons 3, 5, and 29. Measurements of STAG1 expression in HEK 293T cells following 7 days of treatment with 10 μM of ASOs showed a reduction of STAG1 expression was greatest using ASO 3-5, 5-11, and 5-12, with reduction of expression between 50-75% (FIG. 10B).

### Example 9

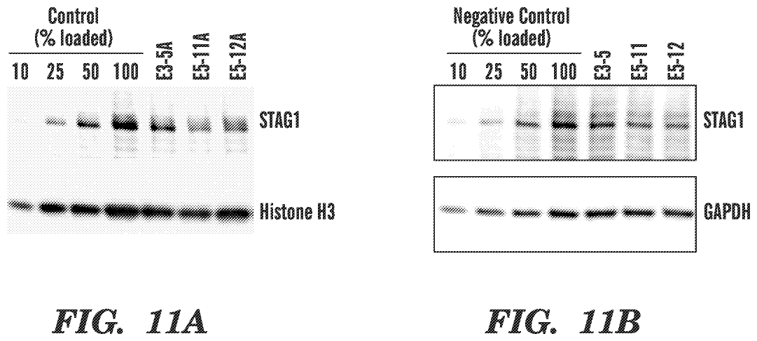

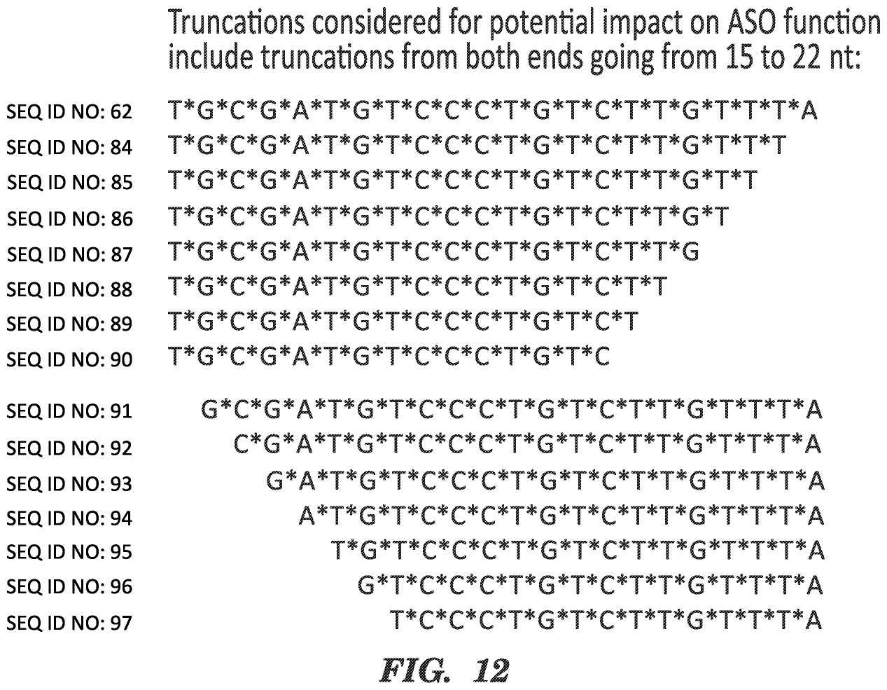

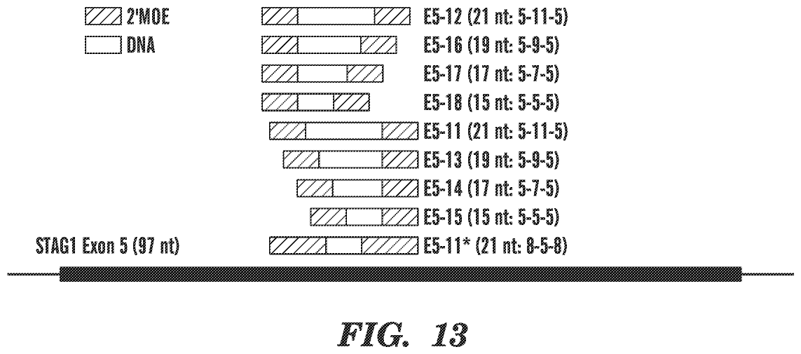

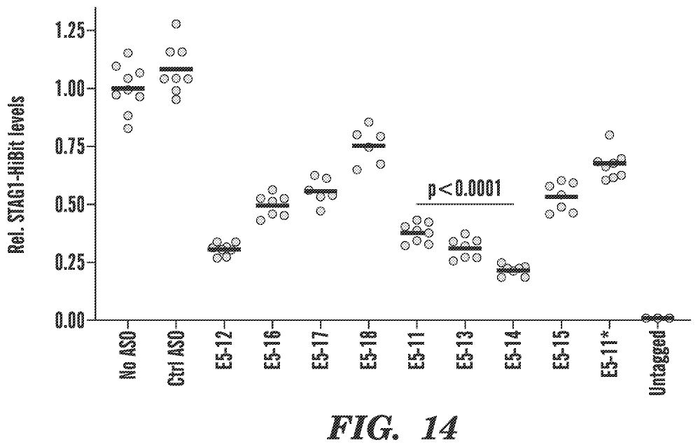

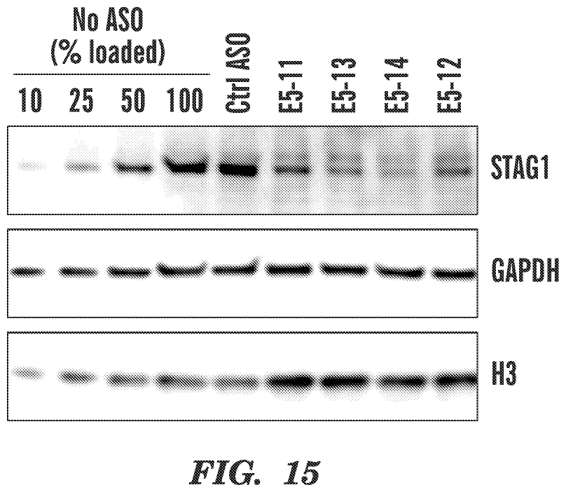

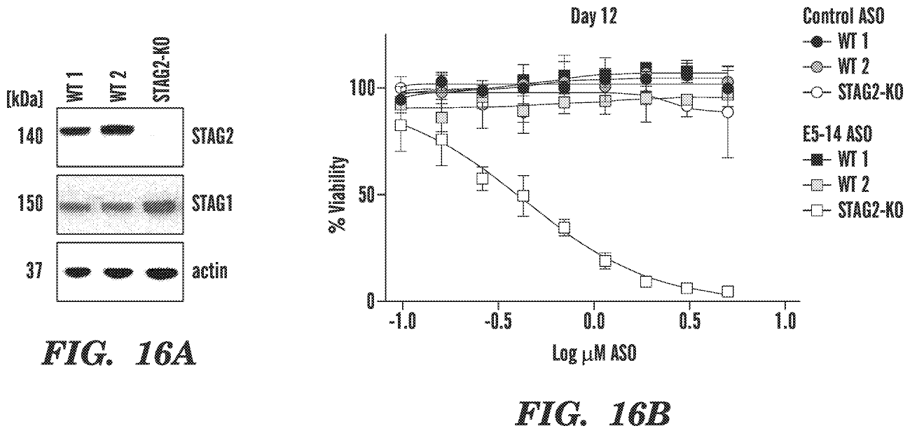

The inventors show reduction of STAG1 protein expression following treatment with STAG1 targeting ASOs. STAG1 protein levels as measured by Western blots. HEK 293T cells were treated with ASOs (E3-5, E5-11, and E5-12) or negative controls for 7 days at a concentration of 10 μM with histone H3 was used as a loading control show that ASOs successfully reduced STAG1 protein level in HEK 293T cells (FIG. 11A). FIG. 11B show STAG1 protein levels as measured by Western blots. U937 AML cells were treated with ASOs (E3-5, E5-11, and E5-12) or negative controls for 7 days at a concentration of 10 μM. GAPDH was used as a loading control. Data indicate that ASOs successfully reduced STAG1 protein level in an AML cell line.

The inventors use a HiBit readout using STAG1-HiBit expressing HEK 293T cells to show potentcy of STAG1 targeting ASOs. Following 4 days of treatment with 10 μM ASOs, the relative STAG1-HiBit levels were measured. E5-14 was the most potent ASO.

### Example 10

The inventors show STAG1 protein levels as measured by Western blots were reduced by STAG1 targeting ASOs. K562 AML cells were treated with ASOs (E5-11, E5-13, E5-14, and E5-12) or negative controls for 4 days at a concentration of 10 μM. GAPDH was used as a loading control. Data indicate that ASOs successfully reduced STAG1 protein level in an AML cell line with E5-14 showing greatest potency. ASOs also successfully increased H3 protein level in an AML cell line.

### Example 11

The inventors show optimized ASO E5-14 causes selective lethality in STAG2 KO AML cells. STAG2 protein levels as measured by Western blots show that STAG2-knock out cells have no STAG2 protein but unaffected levels of STAG1 and actin proteins (FIG. 14A). Measurements of cell viability 12 days following treatment with increasing concentration of control or E5-14 show that ASOs E5-14 did not affect viability in STAG2 proficient cells; however, in STAG2 KO cells, E5-14 ASO greatly reduced cell viability (FIG. 14B). In conclusion, free uptake yields consistent, long-lasting delivery of ASOs in AML cells. Inventors have designed ASOs that achieve considerable reduction in STAG1 mRNA and protein in 293T cells.

1. A composition for downregulating the expression of Stromal Antigen 1 (STAG1), the composition comprising an antisense oligonucleotide having sequence sufficiently complementary to a portion of a STAG1 primary transcript to permit hybridization thereto, wherein the oligonucleotide comprises at least one phosphorothioate backbone modification.

2. The composition of claim 1, wherein the antisense oligonucleotide comprises an RNA oligonucleotide, a DNA oligonucleotide, or a combination of deoxyribonucleosides and ribonucleosides.

3. The composition of claim 1 or claim 2, wherein the antisense oligonucleotide comprises at least one deoxyribonucleoside and at least one ribonucleoside.

4. A composition for downregulating the expression of Stromal Antigen 1 (STAG1), the composition comprising an antisense oligonucleotide having sequence sufficiently complementary to a portion of a STAG1 primary transcript to permit hybridization thereto, wherein the oligonucleotide comprises at least one deoxyribonucleoside and at least one ribonucleoside.

5. The composition of any one of claims 1-4, wherein the antisense oligonucleotide comprises, in 5′ to 3′ order, 1-5 ribonucleosides, 6-12 deoxyribonucleosides, and 1-5 ribonucleosides.

6. The composition of any one of claims 1-5, wherein one or more of the ribonucleosides comprises a 2′-O-methoxyethyl (MOE) modification.

7. The composition of any one of claims 4-6, wherein the antisense oligonucleotide comprises at least one phosphorothioate backbone linkage.

8. The composition of any one of claims 1-7, wherein the antisense oligonucleotide comprises phosphorothioate bonds between each nucleoside.

9. The composition of any one of claims 1-8, wherein the antisense oligonucleotide comprises 15 to 22 nucleosides.

10. The composition of any one of claims 1-9, wherein the antisense oligonucleotide comprises one or more sugar modifications selected from 2′-fluoro, 2′-O-methyl, LNA modification, cEt modification, and PMO modification.

11. The composition of any one of claims 1-10, wherein the antisense oligonucleotide comprises sequence permitting hybridization to exon 3 or 5 of the STAG1 RNA transcript.

12. The composition of any one of claims 1-10, wherein hybridization of the antisense oligonucleotide overlaps a 5′ splice site or an exonic splicing enhancer on the STAG1 RNA transcript.

13. The composition of claim 12, wherein the exonic splicing enhancer comprises a binding site for RNA binding protein SRSF2.

14. The composition of any one of claims 1-13, wherein the antisense oligonucleotide comprises a sequence selected from SEQ ID NOs 1-81.

15. A pharmaceutical formulation comprising the composition of any one of claims 1-14 and a pharmaceutically acceptable carrier.

16. A method of downregulating the expression of STAG1 in a cell, the method comprising contacting the cell with a composition of any one of claims 1-15.

17. The method of claim 16, wherein the cell is a cohesin mutant cell.

18. The method of claim 16 or 17, wherein the cell is a Stromal Antigen 2 (STAG2) mutant cell.

19. The method of any one of claims 16-18, wherein the cell is a cancer cell or a myelodysplastic syndrome cell.

20. The method of any one of claims 16-19, wherein the cell is a cancer cell selected from an acute myeloid leukemia (AML) cell, a glioblastoma cell and a bladder cancer cell.

21. The method of any one of claims 16-20, wherein the contacting reduces STAG1 mRNA by at least 50% in the cell.

22. A method of selectively killing a cohesin mutant cell, the method comprising contacting the cell with a composition of any one of claims 1-15.

23. The method of claim 22, wherein the cell is a STAG2 mutant cell.

24. The method of claim 22 or 23, wherein the cell is a cancer cell or a myelodysplastic syndrome cell.

25. The method of any one of claims 22-24, wherein the cell is a cancer cell selected from an acute myeloid leukemia (AML) cell, a glioblastoma cell and a bladder cancer cell.

26. The method of any one of claims 22-25, wherein the contacting reduces STAG1 mRNA by at least 50% in the cell.

27. A method for treating cohesin mutant cancer, the method comprising administering a composition of any one of claims 1-15 to a subject in need thereof.

28. The method of claim 27, wherein the cohesin mutant cancer is STAG2 mutant.

29. The method of claim 27 or 28, wherein the cohesin mutant cancer is selected from AML, glioblastoma, and bladder cancer.

30. A method of treating myelodysplastic syndrome, the method comprising administering a composition of any one of claims 1-15 to a subject in need thereof.

31. The method of claim 30, wherein myelodysplastic syndrome cells are cohesin mutant.

32. The method of claim 30 or 31, wherein myelodysplastic syndrome cells are STAG2 mutant.

33. The method of any one of claims 27-32, further comprising administering an inhibitor of the DNA damage response.

34. The method of claim 33, wherein the inhibitor of the DNA damage response is a poly (ADP)-ribose polymerase (PARP) inhibitor.

35. The method of claim 33 or 34, wherein the inhibitor of the DNA damage response is selected from talazoparib, veleparib, pamiparib, olaparib, rucaparib and niraparib.
